(19) United States
(12) Patent Application Publication
(10) Pub. No.: US 2020/0407750 A1
(43) Pub. Date: Dec. 31, 2020

(54) NOVEL ADENO-ASSOCIATED VIRUS (AAV) VECTORS, AAV VECTORS HAVING REDUCED CAPSID DEAMIDATION AND USES THEREFOR

(71) Applicant: The Trustees of the University of Pennsylvania, Philadelphia, PA (US)

(72) Inventors: James M. Wilson, Philadelphia, PA (US); April Tepe, Columbia, MD (US); Kevin Turner, Newtown Square, PA (US); Joshua Joyner Sims, Philadelphia, PA (US)

(21) Appl. No.: 16/975,541

(22) PCT Filed: Feb. 27, 2019
(86) PCT No.: PCT/US2019/019804

§ 371 Date: Aug. 25, 2020

Related U.S. Application Data
(60) Provisional application No. 62/635,964, filed on Feb. 27, 2018.
(60) Provisional application No. 62/667,585, filed on May 29, 2018.
(60) Provisional application No. 62/677,471, filed on May 29, 2018.
(60) Provisional application No. 62/703,670, filed on Jul. 26, 2018.
(60) Provisional application No. 62/722,382, filed on Aug. 24, 2018.

(51) Int. Cl. C12N 15/86; C12N 7/00

(52) U.S. Cl. CPC C12N 15/86; C12N 2750/14143; C12N 2750/14121; C12N 7/00

(57) ABSTRACT

A recombinant adeno-associated virus (rAAV) vector comprising an AAV capsid having a heterogeneous population of vp1 proteins, a heterogeneous population of vp2 protein and a heterogeneous population of vp3 proteins. The capsid contains modified amino acids as compared to the encoded VP1 amino acid sequence, the capsid containing highly deamidated asparagine residues at asparagine-glycine pair, and further comprising multiple other, less deamidated asparagine and optionally glutamine residues. Methods of reducing deamidation in the AAV capsid of a rAAV are provided.

### STATEMENT OF FEDERALLY SPONSORED RESEARCH

This invention was made with government support under grant number P01HL059407 awarded by the National Institute of Health's National Heart, Lung, and Blood Institute. The government has certain rights in the invention.

### BACKGROUND OF THE INVENTION

The adeno-associated virus (AAV) capsid is icosahedral in structure and is comprised of 60 of viral protein (VP) monomers (VP1, VP2, and VP3) in a 1:1:10 ratio (Xie Q, et al. Proc Natl Acad Sci USA. 2002; 99(16):10405-10). The entirety of the VP3 protein sequence (519aa) is contained within the C-terminus of both VP1 and VP2, and the shared VP3 sequences are primarily responsible for the overall capsid structure. Due to the structural flexibility of the VP1NP2 unique regions and the low representation of VP1 and VP2 monomers relative to VP3 monomers in the assembled capsid, VP3 is the only capsid protein to be resolved via x-ray crystallography (Nam H J, et al. J Virol. 2007; 81(22):12260-71). VP3 contains nine hypervariable regions (HVRs) that are the primary source of sequence variation between AAV serotypes (Govindasamy L, et al. J Virol. 2013; 87(20):11187-99). Given their flexibility and location on the capsid surface, HVRs are largely responsible for interactions with target cells as well as with the immune system (Huang L Y, et al. J Virol. 2016; 90(11):5219-30; Raupp C, et al. J Virol. 2012; 86(17):9396-408). While the structures of a number of serotypes are published (Protein Data Bank (PDB) IDs 1LP3, 4RSO, 4V86, 3UX1, 3KIC, 2QA0, 2G8G from the Research Collaboratory for Structural Bioinformatics (RCSB) database) for the structure entries for AAV2, AAVrh.8, AAV6, AAV9, AAV3B, AAV8, and AAV4, respectively), there is very little information in the literature regarding modifications on the surface of these capsids. Research suggests that intracellular phosphorylation of the capsid occurs at specific tyrosine residues (Zhong L, et al. Virology. 2008; 381(2):194-202). Despite putative glycosylation sites in the primary VP3 sequence, no glycosylation events have been identified in AAV2(Murray S, et al. J Virol. 2006; 80(12):6171-6; Jin X, et al. Hum Gene Ther Methods. 2017; 28(5):255-267); other AAV serotypes have not yet been evaluated for capsid glycosylation.

AAV gene therapy vectors have undergone less of the molecular-level scrutiny that typically accompanies the development and manufacturing of recombinant protein therapeutics. AAV capsid post-translational modifications (PTM) have largely been unexplored, so accordingly, little is known about their potential to impact function, or about strategies to control PTM levels in manufactured AAV therapies.

Variations in post-translational modifications of non-gene therapy protein therapeutics have complicated their development as drugs. Jenkins, N, Murphy, L, and Tyther, R (2008). Post-translational modifications of recombinant proteins: significance for biopharmaceuticals. Mol Biotechnol 39: 113-118; Houde, D, Peng, Y, Berkowitz, S A, and Engen, J R (2010). Post-translational modifications differentially affect IgG1 conformation and receptor binding. Mol Cell Proteomics 9: 1716-1728. For example, deamidation of selected amino acids modulates the stability of and the immune response to the recombinant protective antigen-based anthrax vaccine. (Powell B S, et al. Proteins. 2007; 68(2):45879; Verma A, et al. Clin Vaccine Immunol. 2016; 23(5):396-402). In some instances, this process is catalyzed by viral or bacterial deamidases to modulate host cell signaling pathways or innate immune responses (Zhao J, et al. J Virol. 2016; 90(9):4262-8; Zhao J, et al. Cell Host Microbe. 2016; 20(6):770-84). More commonly, endogenous deamidation is an enzyme-independent spontaneous process. Although the purpose of spontaneous deamidation has not been fully elucidated, previous studies have suggested that this event serves as a molecular clock to indicate the relative age of a protein and regulate its turnover (Robinson N E and Robinson A B. Proc Natl Acad Sci USA. 2001; 98(3):944-9).

Deamidation occurs when the amide group of asparagine or less frequently glutamine undergoes nucleophilic attack from an adjacent nitrogen atom and the amide group is lost. This process leads to a succinimidyl intermediate (Yang H and Zubarev R A. Electrophoresis. 2010; 31(11):1764-72) that, via hydrolysis, resolves into a mixture of aspartic acid and isoaspartic acid (or glutamic acid and isoglutamic acid) (Catak S, et al. J Phys Chem A. 2009; 113(6):1111-20). Studies of short, synthetic peptides estimate that this hydrolysis results in a 3:1 mixture of isoaspartic acid to aspartic acid (Geiger T. and Clarke S. J Biol Chem. 1987; 262(2):785-94.

There continues to be a need for compositions comprising AAV-based constructs for delivery of heterologous molecules which have stable receptor binding and/or stable capsids, avoid neutralizing antibodies and/or retain purity on storage.

### SUMMARY OF THE INVENTION

In one embodiment, a composition is provided which comprise a mixed population of recombinant adeno-associated virus (rAAV), each of said rAAV comprising: (a) an AAV capsid comprising about 60 capsid vp1 proteins, vp2 proteins and vp3 proteins, wherein the vp1, vp2 and vp3 proteins are: a heterogeneous population of vp1 proteins which are produced from a nucleic acid sequence encoding a selected AAV vp1 amino acid sequence, a heterogeneous population of vp2 proteins which are produced from a nucleic acid sequence encoding a selected AAV vp2 amino acid sequence, a heterogeneous population of vp3 proteins which produced from a nucleic acid sequence encoding a selected AAV vp3 amino acid sequence, wherein: the vp1, vp2 and vp3 proteins contain subpopulations with amino acid modifications comprising at least two highly deamidated asparagines (N) in asparagine-glycine pairs in an AAV capsid and optionally further comprising subpopulations comprising other deamidated amino acids, wherein the deamidation results in an amino acid change; and (b) a vector genome in the AAV capsid, the vector genome comprising a nucleic acid molecule comprising AAV inverted terminal repeat sequences and a non-AAV nucleic acid sequence encoding a product operably linked to sequences which direct expression of the product in a host cell. A mixed population of rAAV results from a production system using a single type of AAV capsid nucleic acid sequence encoding a predicted AAV VP1 amino acid sequence of one AAV type. However, the production and manufacture process provides the heterogenous population of capsid proteins described above. In certain embodiments, the composition is as described in this paragraph, with the proviso that the rAAV is not AAVhu68. In certain embodiments, the composition is as described in this paragraph, with the proviso that the rAAV is not AAV2.

In certain embodiments, the deamidated asparagines are deamidated to aspartic acid, isoaspartic acid, an interconverting aspartic acid/isoaspartic acid pair, or combinations thereof. In certain embodiments, the capsid further comprises deamidated glutamine(s) which are deamidated to (α)-glutamic acid, γ-glutamic acid, an interconverting (α)-glutamic acid/γ-glutamic acid pair, or combinations thereof.

In certain embodiments, a method for reducing deamidation of an AAV capsid is provided. Such method comprises producing an AAV capsid from a nucleic acid sequence containing modified AAV vp codons, the nucleic acid sequence comprising independently modified glycine codons at one to three of the asparagine-glycine pairs relative to a reference AAV vp1 sequence, such that the modified codon encodes an amino acid other than glycine.

In other embodiments, a method for reducing deamidation of an AAV capsid is provided. Such method comprises producing an AAV capsid from a nucleic acid sequence containing modified AAV vp codons, the nucleic acid sequence comprising independently modified asparagine codons of at least one asparagine-glycine pair relative to a reference AAV vp1 sequence, such that the modified codon encodes an amino acid other than asparagine.

A method for increasing the titer, potency, and/or transduction efficiency of an AAV is provided. The method comprises producing an AAV capsid from a nucleic acid sequence containing at least one AAV vp codon modified to change the asparagine or glycine of at least one asparagine-glycine pairs in the capsid to a different amino acid. In certain embodiments, the modified codon(s) is/are in the v2 and/or vp3 region. In certain embodiments, the asparagine-glycine pair in the vp1-unique region is retained in the modified rAAV. In certain embodiments, a nucleic acid molecule sequences encoding these mutant AAV capsids are provided.

In certain embodiments, a deamidation site (e.g., an asparagine-glycine pair or a Gin) is modified at a location other than: (a) N57, N263, N385, N514, and/or N540 of SEQ ID NO: 6 (encoded AAV8 vp1], based on the numbering of the AAV8 vp1, with the initial M, for an AAV8 capsid; (b) N57, N329, N452, and/or N512, based on the numbering of the SEQ ID NO: 7 (encoded AAV9 vp1), with the initial M, for an AAV9 capsid; or (c) N263, N385, and/or N514, based on the numbering of SEQ ID NO: 112 (encoded AAVrh10 vp1), with the initial M, for an AAVrh10 capsid. In certain embodiments, the modified deamidation site is selected from a site on Table F or Table G. In certain embodiments, the modified deamidation site is selected from a site on Table F or Table G, exclusive of the positions in (a)-(c) above. In certain embodiments, a deamidation site (e.g., an asparagine-glycine pair or a Gln (Q) is modified at a location other than: (a) N57, N383, N512, and/or N718, based on the numbering of SEQ ID NO: 1, based on the numbering of the predicted vp1 amino acid sequence with the initial M, for an AAV1 capsid; (b) N57, N382, N512, and/or N718, with reference to the numbering of SEQ ID NO: 2, based on the numbering of the predicted vp1 amino acid sequence with the initial M, for an AAV3B capsid; (c) N56, N347, N347, and/or N509, with reference to the numbering of SEQ ID NO: 3, based on the numbering of the predicted vp1 amino acid sequence with the initial M, for an AAV5 capsid; (d) N41, N57, N384, and/or N514, with reference to the numbering of SEQ ID NO: 4, based on the numbering of the predicted vp1 amino acid sequence with the initial M, for an AAV7 capsid; (e) N57, N264, N292, and/or N318, with reference to the numbering of SEQ ID NO: 5, based on the numbering of the predicted vp1 amino acid sequence with the initial M, for an AAVrh32.33 capsid; or (f) N56, N264, N318, and/or N546, with reference to the numbering of SEQ ID NO: 111, based on the numbering of the predicted vp amino acid sequence with the initial M, for an AAV4 capsid. In certain embodiments, the modified deamidation site is selected from a site on Table A, Table B, Table C, Table D, Table E, Table F, or Table G. In certain embodiments, the modified deamidation site is exclusive of the positions in (a)-(f) listed above.

In certain embodiments, the method involves generating recombinant AAVs having a mutant AAV8 capsid selected having a mutation of: AAV8 G264A/G515A (SEQ ID NO: 21), AAV8G264A/G541A (SEQ ID NO: 23), AAV8G515A/G541A (SEQ ID NO: 25), or AAV8 G264A/G515A/G541A (SEQ ID NO: 27), AAV8 G264A/G541A/N499Q (SEQ ID NO: 115); (c) AAV8 G264A/G541A/N459Q (SEQ ID NO: 116); (d) AAV8 G264A/G541A/N305Q/N459Q (SEQ ID NO: 117); (e) AAV8 G264A/G541A/N305Q/N499Q (SEQ ID NO: 118); AAV8 G264A/G541A/N459Q/N499Q (SEQ ID NO: 119); or AAV8 G264A/G541A/N305Q/N459Q/N499Q (SEQ ID NO: 120); based on the numbering of AAV8 or in another AAV based on alignment of a selected sequence with AAV8. In certain embodiments, the method involves generating rAAV having a mutant AAV9 capsid selected from: AAV9 G330/G453A (SEQ ID NO: 29), AAV9G330A/G513A (SEQ ID NO: 31), AAV9G453A/G513A (SEQ ID NO 33), and/or AAV9 G330/G453A/G513A (SEQ ID NO: 35).

In certain embodiments, a nucleic acid molecule sequences encoding these mutant AAV capsids are provided. In certain embodiments, the nucleic acid sequences are provided in, e.g., SEQ ID NO: 20 (AAV8 G264A/G515A), SEQ ID NO: 22 (AAV8G264A/G541A), SEQ ID NO: 24 (AAV8G515A/G541A), or SEQ ID NO: 26 (AAV8 G264A/G515A/G541A). In certain embodiments, the nucleic acid sequences are provided in, e.g., SEQ ID NO: 28 (9G330AG453A); SEQ ID NO: 30 (9G330AG513A), SEQ ID NO: 32 (9G453AG513A), SEQ ID NO: 34 (9G330AG453AG513A). In certain embodiments, other AAVs may be mutated to have such changes in these or corresponding NG pairs, based on an alignment with AAV9.

A composition comprising a population of rAAV having increased titer, potency, or transduction is provided. In certain embodiments, the composition comprises rAAV having capsids which are modified to have decreased total deamidation as compared to an rAAV with a deamidation pattern with a capsid deamidation pattern according to any one of Table A (AAV1), Table B (AAV3B), Table C (AAV5), Table D (AAV7), Table E (AAVrh32.33), Table F (AAV8), Table G (AAV9), or Table H (AAVhu37). In certain embodiments, the rAAV are unmodified at the highly deamidated positions identified herein.

These and other aspects of the invention will be apparent from the following detailed description of the invention.

### BRIEF DESCRIPTION OF THE DRAWINGS

FIG. 1A-FIG. 1G. Electrophoretic analysis of AAV8 VP isoforms. (FIG. 1A) Diagram illustrating the mechanism by which asparagine residues undergo nucleophilic attack by adjacent nitrogen atoms, forming a succinimidyl intermediate. This intermediate then undergoes hydrolysis, resolving into a mixture of aspartic acid and isoaspartic acid. The beta carbon is labeled as such. The diagram was generated in BIOVIA Draw 2018. (FIG. 1B) 1 μg of AAV8 vector was run on a denaturing one-dimensional SDS-PAGE. (FIG. 1C) Isoelectric points of carbonic anhydrase pI marker spots are shown. (FIG. 1D) 5 μg of AAV8 vector was analyzed by two-dimensional gel electrophoresis and stained with Coomassie Blue. Spots 1-20 are carbamylated carbonic anhydrase pI markers. Boxed regions are as follows: a=VP1, b=VP2, c=VP3, d=internal tropomyosin marker (arrow: tropomyosin spot of MW=33 kDa, pI=5.2). Isoelectric focusing was performed with a pI range of 4-8. FIG. 1E FIG. 1G) Results of isoelectric focusing performed with a pI range of 4-8. 1e11 GC of wtAAV8 (FIG. 1E) or mutant (FIG. 1F and FIG. 1G) vector, which were analyzed by 2D gel electrophoresis and stained with Sypro Ruby. Protein labeling: A=VP1; B=VP2; C=VP3, D=chicken egg white conalbumin marker, E=turbonuclease marker. Isoelectric focusing was performed with a pI range of 6-10. Primary VP1/2/3 isoform spots are circled, and migration distance of major spots of markers are indicated by vertical lines (turbonuclease=dashed, conalbumin=solid).

FIG. 2A-FIG. 2E. Analysis of asparagine and glutamine deamidation in AAV8 capsid proteins. (FIG. 2A-FIG. 2B) Electrospray ionization (ESI) mass spectrometry and theoretical and observed masses of the 3+ peptide (93-103) containing Asn-94 (FIG. 2A) and Asp-94 (FIG. 2B) are shown. (FIG. 2C-FIG. 2D) ESI mass spectrometry and theoretical and observed masses of the 3+ peptide (247-259) containing Asn-254 (FIG. 2C) and Asp-254 (FIG. 2D) are shown. The observed mass shifts for Asn-94 and Asn-254 were 0.982 Da and 0.986 Da, respectively, versus a theoretical mass shift of 0.984 Da. (FIG. 2E) Percent deamidation at specific asparagine and glutamine residues of interest are shown for AAV8 tryptic peptides purified by different methods. Bars indicating deamidation at asparagine residues with N+1 glycines are crosshatched. Residues determined to be at least 2% deamidated in at least one prep analyzed were included. Data are represented as mean standard deviation.

FIG. 3A-FIG. 3E. Structural modeling of the AAV8 VP3 monomer and analysis of deamidated sites. (FIG. 3A) The AAV8 VP3 monomer (PDB identifier: 3RA8) is shown in a coil representation. The color of the ribbon indicates the relative degree of flexibility (blue=most rigid/normal temperature factor, red=most flexible/high temperature factor). Spheres indicate residues of interest. Expanded diagrams are ball and stick representations of residues of interest and their surrounding residues to demonstrate local protein structure (Blue=nitrogen, red=oxygen). Underlined residues are those in NG motifs. FIG. 3B-FIG. 3E: Isoaspartic models of deamidated asparagines with N+1 glycines are shown. The 2FoFc electron density map (1 sigma level) generated from refinement of the AAV8 crystal structure (PDB ID: 3RA8) with (FIG. 3B) an asparagine model of N410 in comparison with isoaspartic acid models of (FIG. 3C) N263, (FIG. 3D) N514, and (FIG. 3E) N540. Electron density map is shown in magenta grid. The beta carbon is labeled as such. Arrow indicates electron density corresponding to the R group of the residue of interest.

FIG. 4A-FIG. 4D. Determination of factors influencing AAV8 capsid deamidation. An AAV8 prep was (FIG. 4A) incubated at 70° C. for three or seven days, (FIG. 4B) exposed to pH 2 or pH 10 for seven days, or (FIG. 4C) prepared for mass spectrometry using D₂O in place of H₂O to determine possible sources of deamidation not intrinsic to AAV capsid formation. (FIG. 4D) A dot blot of vector treated as in FIG. 4A using the B1 antibody (reacts to denatured capsid) and an AAV8 conformation specific antibody (reacts to intact capsids) to assess capsid structural integrity.

FIG. 5A-FIG. 5B. Deamidation frequencies in non-AAV proteins. Deamidation percentages are shown for two non-AAV recombinant proteins containing NG motifs likely to be deamidated, human carbonic anhydrase (FIG. 5A) and rat phenylalanine-hydroxylase (FIG. 5B), for comparison with AAV deamidation percentages.

FIG. 6. Comparison of AAV8 percent deamidation calculated using data analysis pipelines from two institutions. Percent deamidation at specific asparagine and glutamine residues of interest are shown for AAV8 tryptic peptides evaluated at two different institutions.

FIG. 7A-FIG. 7C illustrate functional asparagine substitutions at non-NG sites with high variability between lots. (FIG. 7A) Titers of wtAAV8 and mutant vectors were produced by small-scale triple transfection in 293 cells, as measured by quantitative PCR (qPCR). Titers are reported relative to the wtAAV8 control. Transduction efficiencies were measured as described in FIG. 8B. Titers and transduction efficiencies are normalized to the value for the wtAAV8 control. (FIG. 7B) Representative luciferase images at day 14 post-injection are shown for mice receiving wtAAV8.CB7.ffluc and N499Q capsid mutant vector. (FIG. 7C) Luciferase expression on day 14 of the study periods from C57BL/6 mice injected intravenously with wtAAV8 or mutant vectors (n=3 or 4) was measured by luciferase imaging and reported in total flux units. All data are represented as mean+standard deviation.

FIG. 8A and FIG. 8B show the results of in vitro analysis of the impact of genetic deamidation on vector performance. (FIG. 8A) Titers of wtAAV8 and genetic deamidation mutant vectors produced by small-scale triple transfection in 293 cells, as measured by quantitative PCR (qPCR). Titers are reported relative to the wtAAV8 control. NG sites with high deamidation (patterned bars), sites with low deamidation (white bars) and highly variable sites (black bars) are presented with wtAAV8 and a negative control. (FIG. 8B) Transduction efficiency of mutant AAV8 vectors producing firefly luciferase reported relative to the wtAAV8 control. Transduction efficiency is measured in luminescence units generated per GC added to HUH7 cells, and is determined by performing transductions with crude vector at multiple dilutions. Transduction efficiency data are normalized to the wild-type (wt) reference. All data are represented as mean standard deviation.

FIG. 9A-FIG. 9D illustrate that vector activity loss through time is correlated to progressive deamidation. (FIG. 9A) Vector production (DNAseI resistant Genome Copies, GC) for a timecourse of triple-transfected HEK 293 cells producing AAV8 vector packaging a luciferase reporter gene. GC levels are normalized to the maximum observed value. (FIG. 8B) Purified timecourse vector was used to transduce Huh7 cells. Transduction efficiency (luminescence units per GC added to target cells) was measured as in FIG. 8B using multiple dilutions of purified timecourse vector samples. Error bars represent the standard deviation of at least 10 technical replicates for each sample time. Deamidation of AAV8 NG sites (FIG. 9C) and non-NG sites (FIG. 9D) for vector collected 1, 2 and 5 days post transfection.

FIG. 10A-FIG. 10D illustrates the impact of stabilizing asparagines on vector performance. FIG. 10A shows titers of wtAAV8 and +1 position mutant vectors produced by small-scale triple transfection in 293 cells, as measured by quantitative PCR (qPCR). Titers are reported relative to the wtAAV8 control. FIG. 10B shows the transduction efficiency of mutant AAV8 vectors producing firefly luciferase reported relative to the wtAAV8 control. Transduction efficiency was measured as in FIG. 8B using crude vector material. A two-sample t-test (*p<0.005) was run to determine significance between wtAAV8 and mutant transduction efficiency for G264A/G515A and G264A/G541A. FIG. 10C shows luciferase expression on day 14 of the study period in the liver region from C57BL/6 mice injected intravenously with wtAAV8 or mutant vectors (n=3 to 5) measured by luciferase imaging and reported in total flux units. FIG. 10D shows the titers and transduction efficiency of multi-site AAV8 mutant vectors producing firefly luciferase reported relative to the wtAAV8 control. All data are represented as mean standard deviation.

FIG. 11A-FIG. 11C. Analysis of asparagine and glutamine deamidation in AAV9 capsid proteins. (FIG. 1A) 1e11 GCs of wtAAV9 were analyzed by 2D gel electrophoresis and stained with Sypro Ruby. Protein labeling: A=VP1; B=VP2; C=VP3, D=chicken egg white conalbumin marker, E=turbonuclease marker. Isoelectric focusing was performed with a pI range of 6-10. (FIG. 11B) Percent deamidation at specific asparagine and glutamine residues of interest are shown for AAV9 tryptic peptides purified by different methods. Bars indicating deamidation at asparagine residues with N+1 glycines are crosshatched. Residues determined to be at least 2% deamidated in at least one prep analyzed were included. Data are represented as mean standard deviation. (FIG. 11C) Isoaspartic model of N512 is shown in the 2FoFc electron density map generated by non-biased refinement of the AAV9 crystal structure (PDB ID: 3UX1). Arrow indicates electron density corresponding to the R group of residue N512.

FIG. 11D-FIG. 11F. Determination of factors influencing AAV9 capsid deamidation. (FIG. 11D) Two AAV9 preps were incubated at 70° C. for three or seven days or (FIG. 11F) exposed to pH 2 or pH 10 for seven days to determine possible sources of deamidation not intrinsic to AAV capsid formation. Data are represented as mean standard deviation. (FIG. 11F) A dot blot of vector treated as in FIG. 1D using the B1 antibody (reacts to denatured capsid) to assess capsid structural integrity.

FIG. 11G and FIG. 11H illustrate in vitro analysis of the impact of genetic deamidation on vector performance for AAV9. (FIG. 11G) Titers of wtAAV9 and genetic deamidation mutant vectors were produced by small-scale triple transfection in 293 cells, as measured by quantitative PCR (qPCR). Titers are reported relative to the wtAAV9 control. NG sites with high deamidation (patterned bars), sites with low deamidation (white bars) and highly variable sites (black bars) are presented with wtAAV8 and a negative control. (FIG. 11H) The transduction efficiency of mutant AAV9 vectors producing firefly luciferase are reported relative to the wtAAV9 control. All data are represented as mean standard deviation.

FIG. 11I-FIG. 11K show AAV9 vector in vitro potency through time. (FIG. 11I) Vector production (DNAseI resistant Genome Copies, GC) for a timecourse of triple-transfected HEK 293 cells producing AAV9 vector packaging a luciferase reporter gene. GC levels are normalized to the maximum observed value. (FIG. 11J) Crude timecourse vector was used to transduce Huh7 cells. (FIG. 11K) Transduction efficiencies of vector collected 1 day post transfection vs 5 days post transfection are shown for crude and purified vector samples. Transduction efficiency is expressed as luciferase activity/GC, normalized to the value at day 1.

FIG. 12A-FIG. 12B. Characterization of the PAV9.1 monoclonal antibody and mutagenesis strategy based on the PAV9.1 epitope. FIG. 12A: PAV9.1 recognition of various AAV serotypes based on capture ELISA with native or denatured capsid protein. FIG. 12B: Alignment of AAV VP1 amino acid sequences (SEQ ID NOs: 10-19, top to bottom); residues of interest to the epitope of PAV9.1 are within the black box.

FIG. 13A-FIG. 13D. Cryo-EM reconstruction of AAV9 in complex with PAV9.1 Fab. FIG. 13A: Depiction of the molecular surface of AAV9 capsid (fuchsia) bound with PAV9.1 Fab (blue at the protrusion of the three-fold axis reconstructed at a 4.2 Å resolution. We boxed 3,022 particles and used Auto3dEM for electron microscopy reconstruction. FIG. 13B: Depiction of a cross-section of the AAV9-PAV9.1 complex. FIG. 13C: Pseudo-atomic model of AAV9-PAV9.1 trimer built into density as obtained from cryo-reconstruction. VP3 monomers are shown in green, gray, and cyan. Spheres represent bound residues. We have illustrated a single PAV9.1 Fab with the heavy chain in indigo and the light chain in red. FIG. 13D: Two-dimensional “roadmap” of residues involved in PAV9.1 binding.

FIG. 14A-FIG. 14E. Effect of epitope mutations on the EC50 of PAV9.1 mAb for AAV9. We used capsid capture ELISA against AAV9 to analyze and generate binding curves for PAV9.1. FIG. 14A-FIG. 14E illustrate the following: 586-590 swap mutants (FIG. 14A); 494-498 mutants (FIG. 14B); 586-590 point mutants (FIG. 14C); AAV9.TQAAA and AAV9.SAQAN single and combination mutants (FIG. 14D); AAV9.TQAAA and AAV9.SAQAA single and combination mutants (FIG. 14E). We normalized absorbance to the maximum absorbance for each capsid. We determined the line of best fit and EC50 using the dose response function in Prism.

FIG. 15A-FIG. 15K. Characterizing the impact of PAV9.1 epitope mutations on in vitro vector transduction and effective PAV9.1 mAb neutralizing titer. FIG. 15A: Transduction efficiency of PAV9.1 capsid mutants relative to AAV9.WT in HEK293 cells. We determined significance by using a two-sided one-sample t-test and compared the percent transduction of each mutant to the transduction of AAV9.WT (defined as 100%). P-values indicated as follows: p*<0.05, p***<0.001. FIG. 15B-FIG. 15K: Determining the neutralizing titer of PAV9.1 when transducing HEK293 cells with AAV9.WT.CMV.LacZ (FIG. 15B); AAV9.AAQAA (FIG. 15C); AAV9.QQNAA (FIG. 15D); AAV9.SSNTA (FIG. 15E); AAV9.RGNRQ (FIG. 15F); AAV9.RGHRE (FIG. 15G); AAV9.TQAAA (FIG. 15H); AAV9.AANNN (FIG. 15I); AAV9.SAQAN (FIG. 15J); or AAV9.SAQAA (FIG. 15K). We defined the neutralizing titer as the dilution prior to the time point when we could achieve transduction levels of 50% or greater than the vector without mAb (levels measured in relative light units). All data are reported as mean±SD.

FIG. 16. Correlation between PAV9.1 EC50 and neutralizing titer for a panel of AAV9 mutants. We calculated the fold reduction in PAV9.1 neutralizing titer against each mutant relative to PAV9.1 neutralizing titer against AAV9.WT. We plotted the data on a log scale against the fold increase in PAV9.1 EC50 for each mutant relative to PAV9.1 EC50 for AAV9.WT on a linear scale (semi-log plot). We used GraphPad Prism to determine the semi-log line of best fit; R²=0.8474.

FIG. 17A-FIG. 17G. In vivo analysis of AAV9 PAV9.1 mutant vectors. C57BL/6 mice received intravenous injections of either 1e11 GC per mouse (FIG. 17A-FIG. 17C) or 1e12 GC per mouse (FIG. 17D-FIG. 17F) AAV9.CMV.LacZ (WT or mutant; n=3). We sacrificed mice on day 14 and harvested tissues for biodistribution analysis (FIG. 17A and FIG. 17D) using Taqman qPCR. Values are reported as mean±SD. We also harvested liver (FIG. 17B and FIG. 17E), heart (FIG. 17C and FIG. 17F) and muscle (FIG. 17G) for β-gal histochemistry to determine enzyme activity. Representative 10× images are shown; scale bars=200 μm.

FIG. 18A-FIG. 18D. Effect of epitope mutations on EC50 of injected mouse plasma for AAV9. Using capsid capture ELISA, we analyzed the day-56 plasma of mice that received intravenous injections of either 7.5e8 GC/mouse (FIG. 18A); or 7.5e9 GC/mouse (FIG. 18B) wtAAV9.LSP.hFIX for AAV9.WT or AAV9 PAV9.1 mutant binding. We normalized absorbance to the maximum absorbance achieved for each capsid. We determined the line of best fit and EC50 using the dose response function in Prism. Each graph corresponds to a single animal. We compiled EC50 values for 7.5e8 GC/mouse (FIG. 18C); or 7.5e9 GC/mouse (FIG. 18D) to determine the average for each mutant. A two-sided one-sample t-test was used to determine if there was a significant difference between the EC50 of plasma for each mutant relative to the EC50 of plasma for AAV9.WT (defined as 1). The Bonferroni correction was applied to control for type 1 error. P-values are represented as follows: **=p<0.05, **=p<0.01, ***=p<0.001. EC50 data are reported as mean±SD.

FIG. 19A-FIG. 19D. Effect of epitope mutations on EC50 of NHP polyclonal serum for AAV9. Using capsid capture ELISA, we analyzed the sera from (FIG. 19A) NHPs treated with AAV9.WT or hu68.WT vector; or (FIG. 19B) naïve NHPs that are AAV9 NAb (+) for AAV9.WT or AAV9 PAV9.1 mutant binding. We normalized the absorbance to the maximum absorbance achieved for each capsid. We used the dose response function in Prism to determine the line of best fit and EC50. Each graph corresponds to a single animal. We compiled EC50 values for vector-treated NHPs (FIG. 19C); and naïve NAb (+) NHPs to determine (FIG. 19D) the average for each mutant. We used a two-sided one-sample t-test to determine if there is a significant difference between the EC50 of plasma for each mutant relative to the EC50 of plasma for AAV9.WT (defined as 1). The Bonferroni correction was applied to control for type 1 error. EC50 data are reported as mean±SD.

FIG. 20A-FIG. 20B. Effect of epitope mutations on EC50 of human donor polyclonal sera for AAV9. FIG. 20A: We analyzed the sera from naïve human donors that were AAV9 NAb (+) for AAV9.WT or AAV9 PAV9.1 mutant binding using capsid capture ELISA. We determined the line of best fit and EC50 using the dose-response function in Prism. Each graph corresponds to a single donor. FIG. 20B: We compiled EC50 values for NAb (+) human donor serum to determine the average for each mutant. We determined significance by using a two-sided one-sample t-test and compared the EC50 of plasma for each mutant relative to the EC50 of plasma for AAV9.WT (defined as 1). The Bonferroni correction was applied to control for type 1 error. EC50 data are reported as mean±SD.

FIG. 21A-FIG. 21B show AAV8 in vitro titer and transduction data from 6-well plate scale experiments, including N57Q, N263Q, N385Q, N514Q, N540Q, N94Q. and N410Q mutants for AAV8.

FIG. 22A-FIG. 22B show AAV9 in vitro titer and transduction data from 6-well plate scale experiments, including N57Q, N329Q, N452Q, N270Q, N409Q, N668Q, N94Q, N253Q, N663Q, and N704Q mutants for AAV9.

FIG. 23A-FIG. 23B provide in vivo transduction data for AAV8 and AAV9, respectively, in mice tested for liver expression in mice on day 14 (luciferase imaging). FIG. 23A illustrates AAV8 mutants N57Q, N263Q and N385Q, as compared to wild-type for AAV8. FIG. 23B illustrates AAV9 mutants N57Q, G58A, G330A, as compared to wild-type AAV9.

FIG. 24A-FIG. 24B illustrate relative titer (GC) and transduction efficiency for AAV9 double and triple mutants G330/G453A, G330A/G513A, G453A/G513A, and G330/G453A/G513A. FIG. 24A compares relative titer of the mutants to AAV9 wt and FIG. 24B compares relative transduction efficiency (luciferase/GC) of the mutants to AAV9 wt.

### DETAILED DESCRIPTION OF THE INVENTION

Provided herein are recombinant adeno-associated virus (rAAV) having sequence and charge heterogeneity in each of the three populations of capsid proteins VP1, VP2, and VP3 found within the capsid of a recombinant AAV and compositions containing same. Provided herein are novel rAAV, as well as methods for reducing the deamidation, and optionally other capsid monomer modifications. Further provided herein are modified rAAV having decreased modifications, which are useful for providing rAAV having capsids which retain greater stability, potency, and/or purity. In certain embodiments, the rAAV is not AAVhu68. In certain embodiments, the rAAV is not AAV2.

In one embodiment, a composition is provided which comprise a mixed population of recombinant adeno-associated virus (rAAV), each of said rAAV comprising: (a) an AAV capsid comprising about 60 capsid vp1 proteins, vp2 proteins and vp3 proteins, wherein the vp1, vp2 and vp3 proteins are: a heterogeneous population of vp1 proteins which are produced from a nucleic acid sequence encoding a selected AAV vp1 amino acid sequence, a heterogeneous population of vp2 proteins which are produced from a nucleic acid sequence encoding a selected AAV vp2 amino acid sequence, a heterogeneous population of vp3 proteins which produced from a nucleic acid sequence encoding a selected AAV vp3 amino acid sequence, wherein: the vp1, vp2 and vp3 proteins contain subpopulations with amino acid modifications comprising at least two highly deamidated asparagines (N) in asparagine-glycine pairs in an AAV capsid and optionally further comprising subpopulations comprising other deamidated amino acids, wherein the deamidation results in an amino acid change; and (b) a vector genome in the AAV capsid, the vector genome comprising a nucleic acid molecule comprising AAV inverted terminal repeat sequences and a non-AAV nucleic acid sequence encoding a product operably linked to sequences which direct expression of the product in a host cell. In certain embodiments, the composition is as described in this paragraph, with the proviso that the rAAV is not AAVhu68. As used herein, AAVhu68 is as define din WO 2018/160582. The predicated amino acid sequence of the AAVhu68 VP1 is reproduced in SEQ ID NO: 114 and the native nucleic acid sequence is provided n SEQ ID NO: 113. In certain embodiments, the composition is as described in this paragraph, with the proviso that the rAAV is not AAV2.

In certain embodiments, the mixed population of rAAV results from a production system using a single AAV capsid nucleic acid sequence encoding a predicted AAV VP1 amino acid sequence of one AAV type. However, the production and manufacture process provides the heterogenous population of capsid proteins described above.

In certain embodiments, recombinant AAVs are produced having mutant AAV8 capsids having one or more improved property relative to the unmodified AAV8 capsid are provided. Such improved properties may include, for example, increased titer and/or increased relative transduction efficiency as compared to AAV8. In certain embodiments, mutants may include AAV8 G264A/G515A (SEQ ID NO: 21), AAV8G264A/G541A (SEQ ID NO: 23), AAV8G515A/G541A (SEQ ID NO: 25), or AAV8 G264A/G515A/G541A (SEQ ID NO: 27). In certain embodiments, nucleic acid sequences encoding these mutant AAV8 capsids are provided. In certain embodiments, the nucleic acid sequences are provided in, e.g., SEQ ID NO: 20 (AAV8 G264A/G515A), SEQ ID NO: 22 (AAV8G264A/G54A), SEQ ID NO: 24 (AAV8G515A/G541A), or SEQ ID NO: 26 (AAV8 G264A/G515A/G541A). In certain embodiments, an AAV8 mutant may be N499Q, N459Q, N305Q/N459Q, N305QN499Q, N459Q, N305Q/N459Q, N305q/N499Q, or N205Q, N459Q, or N305Q/N459Q, N499Q. In certain embodiments, these mutations are combined with a G264A/G541A mutation. In certain embodiments, the mutation is AAV8 G264A/G541A/N499Q (SEQ ID NO: 115); AAV8 G264A/G541A/N459Q (SEQ ID NO: 116); AAV8 G264A/G541A/N305Q/N459Q (SEQ ID NO: 117); AAV8 G264A/G541A/N305Q/N499Q (SEQ ID NO: 118); G264A/G541A/N459Q/N499Q (SEQ ID NO: 119); or AAV8 G264A/G541A/N305Q/N459Q/N499Q (SEQ ID NO: 120). In other embodiments, single mutants, such as AAV8N263A, AAV8N514A, AAV8N540A or may selected. In certain embodiments, other AAVs may be mutated to have changes in these or corresponding NG pairs, based on an alignment with AAV8. Such AAVs may be clade E AAVs. See, for example, an AAV8 mutant described in Example 2 (SEQ ID NO:9).

In certain embodiments, AAV8 mutants avoid changing the NG pairs at positions N57, N94, N263, N305, G386, Q467, N479, and/or N653. In certain embodiments, other AAVs avoid mutation at corresponding N positions as determined based on an alignment with AAV8, using AAV8 numbering as a reference.

In certain embodiments, recombinant AAVs are produced having mutant AAV9 capsids having one or more improved property relative to the unmodified AAV9 capsid are provided. Such improved properties may include, for example, increased titer and/or increased relative transduction efficiency as compared to AAV9. In certain embodiments, mutant AAV9 capsids may include, e.g., AAV9 G330/G453A (SEQ ID NO: 29), AAV9G330A/G513A (SEQ ID NO: 31), AAV9G453A/G513A (SEQ ID NO 33), and/or AAV9 G330/G453A/G513A (SEQ ID NO: 35). In certain embodiments, nucleic acid sequences encoding these mutant AAV9 capsids are provided. In certain embodiments, the nucleic acid sequences are provided in, e.g., SEQ ID NO: 28 (9G330AG453A); SEQ ID NO: 30 (9G330AG513A), SEQ ID NO: 32 (9G453AG513A), SEQ ID NO: 34 (9G330AG453AG513A). In certain embodiments, other AAVs may be mutated to have such changes in these or corresponding NG pairs, based on an alignment with AAV9. Such AAVs may be clade F AAVs.

In certain embodiments, rAAVs having mutant AAV capsids of Clade A, Clade B, Clade C or Clade D may be engineered to have an amino acid modifications of the NG pairs corresponding to those identified above for Clade E and Clade F. In certain embodiments, Clade A (e.g., AAV) mutants may include mutations at positions N303, N497, or N303/N497, with reference to the numbering of SEQ ID NO: 1 (AAV1). In certain embodiments, the mutant is N497Q. In certain embodiments, AAV3B mutants may include mutations at positions N302, N497, or N302/N497, with reference to the numbering of SEQ ID NO: 2. In certain embodiments, the mutant is N497Q. In certain embodiments, AAV5 mutants may include mutations at positions N302, N497, or N302/N497, with reference to the numbering of SEQ ID NO: 3. In certain embodiments, the mutant is N497Q.

Without wishing to be bound by theory, mass spectrometry revealed deamidation of asparagine at a number of locations on the capsid as an explanation for the presence of multiple VP isoforms, which has not been previously described for AAV. Additionally, the distribution and extent of deamidation was consistent across a number of methods of vector purification, suggesting this phenomenon occurs independently of vector processing. The functional significance of these deamidations was interrogated by individually mutating some asparagines to aspartic acids. A subset of these mutations impacted not only the efficiency of particle assembly but also the ability of the vector to transduce target cells both in vitro and in vivo. De novo modeling of these deamidated residues into the AAV8 structure also revealed structural evidence for the presence of these deamidation events and provided a computational explanation for why the AAV8 capsid tolerates these changes in amino acid identity and properties. Virtually identical findings of deamination were seen with AAV9, and a variety of additional AAVs. Thus, rAAV are characterized by a previously unknown AAV capsid structural heterogeneity.

In the studies reported herein, we found widespread asparagine and occasional glutamine deamidation with 17 residues affected. Factors controlling AAV8 deamidation, principally primary-sequence and 3D structural constraints, are likely conserved across the entire AAV phylogeny, as all serotypes analyzed by us to date exhibit a strikingly similar pattern of modification. Thus deamidation is a potentially critical factor in the development of all future AAV therapeutics.

With this discovery, we were motivated to explore the functional impacts of AAV deamidation. The multimeric nature of AAV vector capsids, the extent and number of modified capsid residues, and the resulting mosaic diversity in vector particle compositions presented some special challenges to this analysis. The experimental repertoire that might sufficiently parameterize post-translational modification (PTM) impact in a simpler protein context did not apply directly to AAV capsid analysis. For example, it would be impossible to purify or even enrich a preparation for a particular deamidated vector species to test its function directly and in isolation.

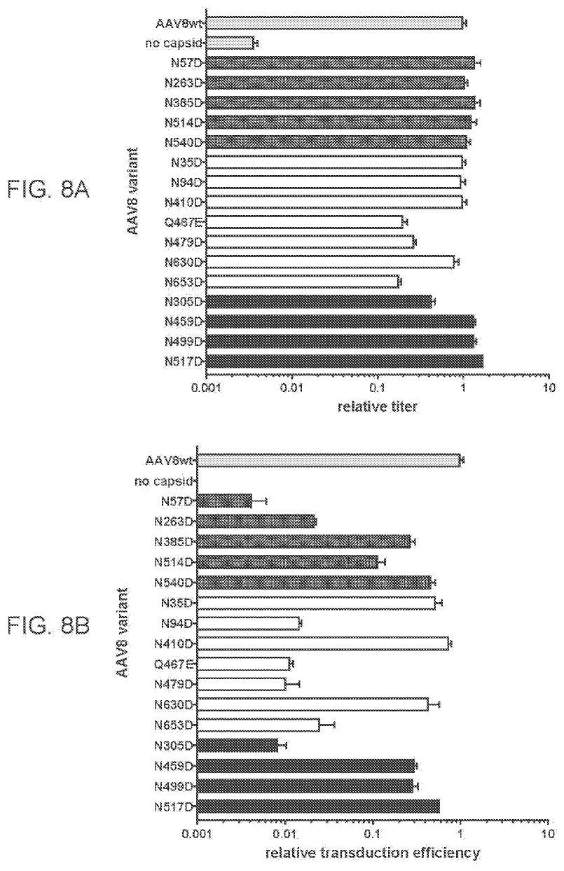

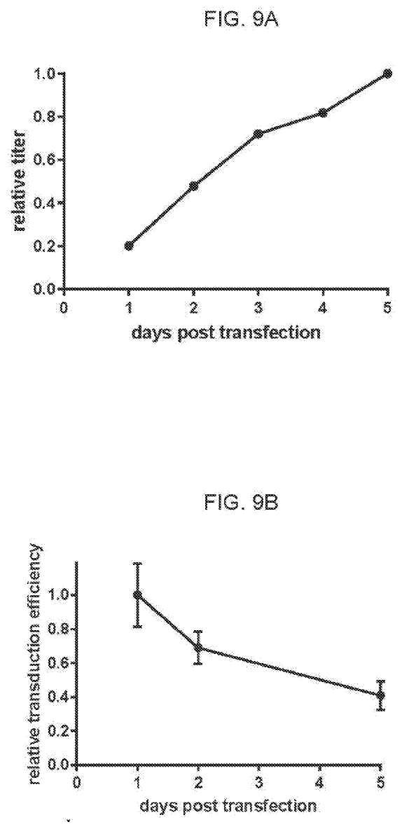

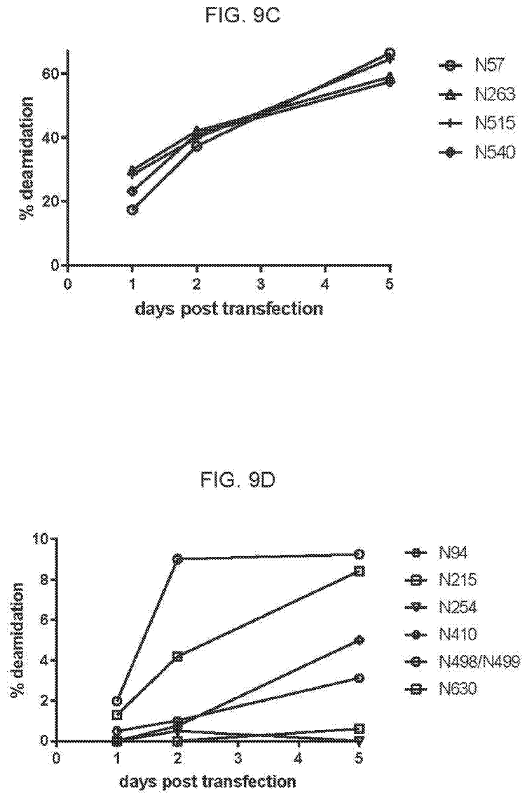

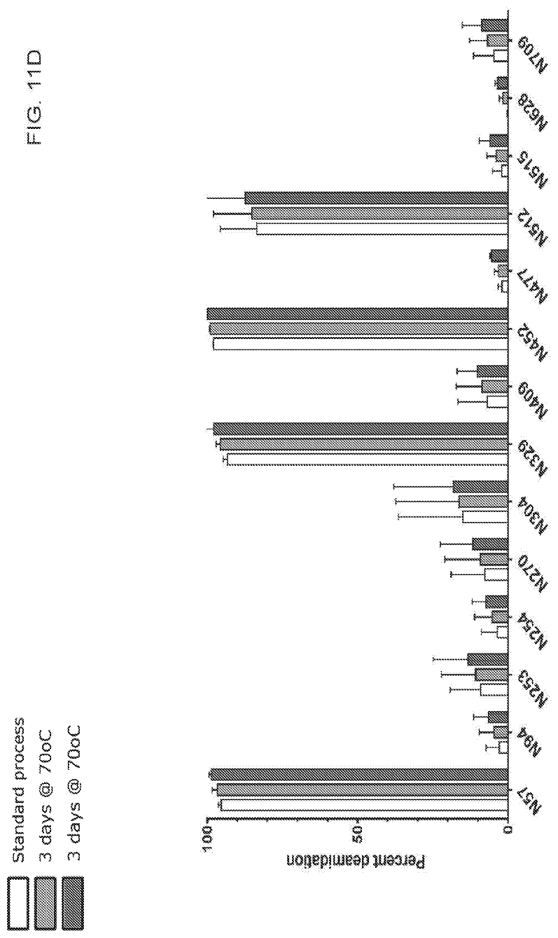

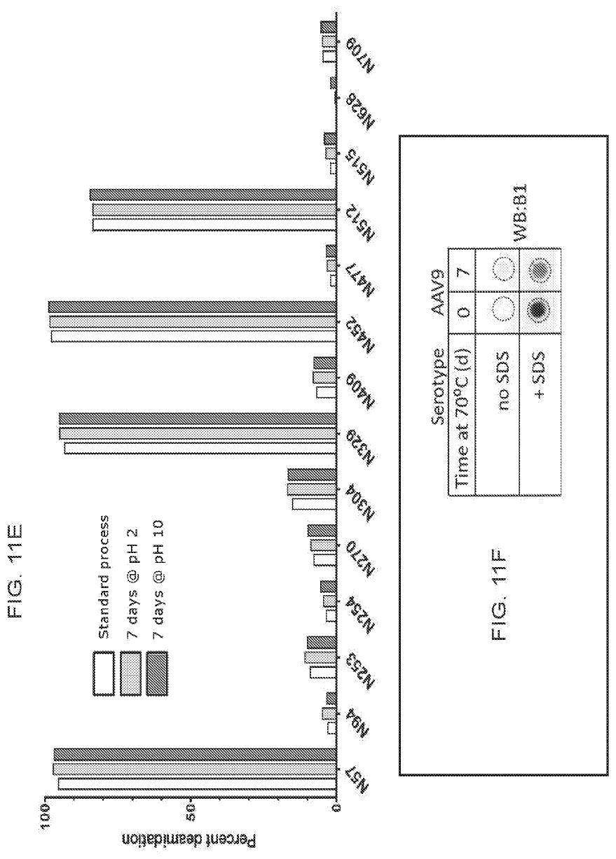

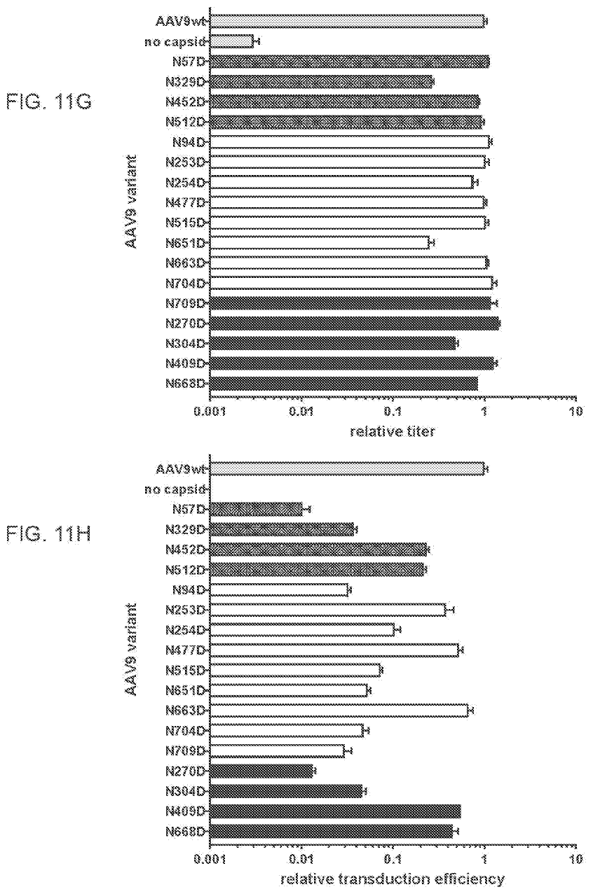

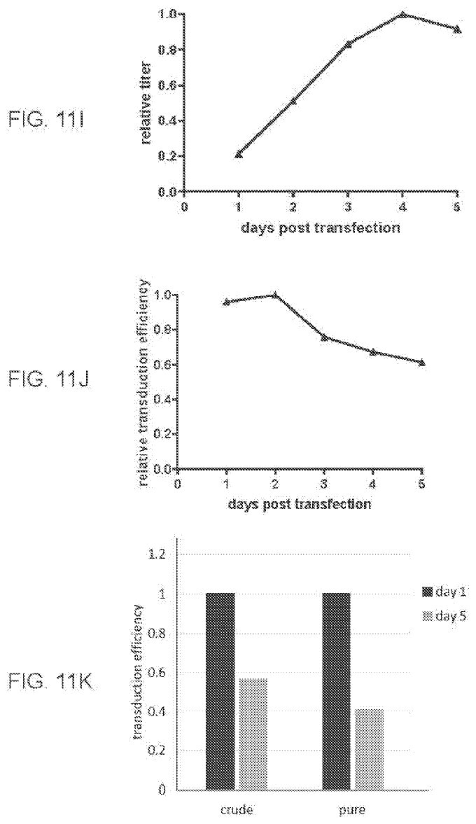

Genetic substitution to aspartate is one approach we tried to force an approximation of the modification at a given site. Beyond the previously noted differences between the distributions of position-specific modification on capsid assemblies with endogenous (mosaic) vs genetic (complete) deamidation, our data points out additional considerations for interpreting this data. For example, we observed >50-fold transduction loss for the N263D mutant relative to the wtAAV8 (FIG. 8B). This was surprising given that the change in aspartate content at this position upon genetic conversion would be marginal; N263 is deamidated at 99% in wtAAV8. One explanation for this discrepancy is that genetically encoded aspartate and the product of asparagine deamidation are molecularly distinct (L-aspartate vs a presumed 3:1 mixture of L/D-isoaspartate: L/D-aspartate). Thus the genetic approximation may be insufficient at some positions. Another residue, the highly conserved N57, was also not tolerant of substitution to aspartate, though it was on average 80% and 97% deamidated in AAV8 and AAV9, respectively (FIGS. 8B and 11). Here, the residual intact amides may buffer the activity of the wt preparations through mosaic effects, though we also detected the potential for cross-talk with other asparagines to confound analysis of N57; neighboring N66 became significantly deamidated when the position 57 amide was preserved mutagenically (N57Q, G58A, and G58S for AAV8; N57Q and G58A for AAV9; data not shown). This was the only case of cross-talk apparent we detected from mass spectrometry analysis of our mutants, but it highlights another complication of interpreting our loss-of-function mutagenic data.

Given these caveats, we developed evidence of the impact of deamidation through time course and gain-of-function mutagenesis experiments. Our data is consistent with a role for a subset of NG sites in the functional loss associated with very early timepoint deamidation. To our knowledge, this phenomenon has not been previously reported. Indeed, the particular experimental procedure we used to identify this decay was informed by the novel observation of very short half-life vector NG deamidation; storing early samples for even a single day in the refrigerator would likely diminish their distinction from late timepoint samples given the speed of spontaneous deamidation we observed. Storage stability experiments comparing the activity of vector preparations over days or weeks after processing are routine at our lab and other manufacturing groups, but these comparisons are almost always made with vector material that is at least 7 days old, when most or all of the activity decay (and NG site deamidation) is complete. The data highlights an opportunity for process interventions or N-stabilizing mutagenic approaches to yield improved capsids. From a broader perspective, it is of interest as well to consider the role of a “deamidation clock” in the natural ecology of AAVs, where this phenomenon would presumably advantage the most recently translated virus particles from an infected cell for the next round of infection.

We did not explore the mechanistic underpinnings of NG deamidation-induced functional loss, though some prominent possibilities exist. All the NG motifs in AAV8 and AAV9 VP3 are found in surface HVR loops. In AAV8, NG 514 and 540 are located near the 3-fold axis in an area known to play a significant role in transduction due to interactions with cellular receptors. While no AAV8 receptor binding site has been fully interrogated, the LamR receptor has been implicated in AAV8 transduction. These studies identify aa491-557 as important for these interactions. Receptor binding for AAV9 is better characterized than that of AAV8, as functional interrogation of the capsid identified the residues in the AAV9 galactose binding domain. Of these residues, we found a single asparagine, N515, to be deamidated at a low level (3%), while two other asparagines in this domain, N272 and N470, were not found to be deamidated. Therefore, while there is a potential for deamidation to influence galactose binding, it is likely only to a small degree.

In summary, we identified that AAV vector deamidation can impact transduction efficiency, and demonstrated strategies to stabilize amides and improve vector performance. A key future goal will be to extend these findings to appropriate animal model systems, and begin to consider the impact of deamidation and the performance of our stabilized variants in more complex functional contexts. Tissue tropism and interactions of the capsid with the immune system could be impacted and must be evaluated carefully. Because these complex effects will likely be very difficult to determine conclusively for every deamidated residue in the capsid, it may be prudent to target the limited number of residues with high lot-to-lot variability in deamidation for stabilization through mutagenesis, as we have demonstrated successfully for variable AAV8 asparagines 459 and 499. Additionally, deamidation analysis of vector preparations using our mass spectrometry workflow may prove beneficial in achieving functional consistency in manufactured lots of AAV gene therapy pharmaceuticals.

A “recombinant AAV” or “rAAV” is a DNAse-resistant viral particle containing two elements, an AAV capsid and a vector genome containing at least non-AAV coding sequences packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs may be used. Further, the vector genome contains regulatory sequences which direct expression of the gene products. Suitable components of a vector genome are discussed in more detail herein.

A rAAV is composed of an AAV capsid and a vector genome. An AAV capsid is an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences.

As used herein, the term “heterogeneous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, or up to about 100% deamidated at a referenced amino acid position, as compared to the predicted amino acid sequence at the reference amino acid position (e.g., at least 80% of the asparagines at amino acid 57 based on the numbering of SEQ ID NO: 1 [AAV1], 2 [AAV3B], 4 [AAV7], 5, [AAVrh32.33], 6 [AAV8], 7 [AAV9], 9 [AAV8 triple], or 111 [AAVhu37] or amino acid 56 based on the numbering of SEQ ID NO: 3 [AAV5], may be deamidated based on the total vp1 proteins may be deamidated based on the total vp1, vp2 and vp3 proteins). Such percentages may be determined using 2D-gel, mass spectrometry techniques, or other suitable techniques.

As used herein, a “deamidated” AAV is one in which one or more of the amino acid residues has been derivatized to a residue which differs from that which encodes it in the corresponding nucleic acid sequence.

Without wishing to be bound by theory, the deamidation of at least highly deamidated residues in the vp proteins in the AAV capsid is believed to be primarily non-enzymatic in nature, being caused by functional groups within the capsid protein which deamidate selected asparagines, and to a lesser extent, glutamine residues. Efficient capsid assembly of the majority of deamidation vp1 proteins indicates that either these events occur following capsid assembly or that deamidation in individual monomers (vp1, vp2 or vp3) is well-tolerated structurally and largely does not affect assembly dynamics. Extensive deamidation in the VP1-unique (VP1-u) region (˜aa 1-137), generally considered to be located internally prior to cellular entry, suggests that VP deamidation may occur prior to capsid assembly. The deamidation of N may occur through its C-terminus residue's backbone nitrogen atom conducts a nucleophilic attack to the Asn's side chain amide group carbon atom. An intermediate ring-closed succinimide residue is believed to form. The succinimide residue then conducts fast hydrolysis to lead to the final product aspartic acid (Asp) or iso aspartic acid (IsoAsp). Therefore, in certain embodiments, the deamidation of asparagine (N or Asn) leads to an Asp or IsoAsp, which may interconvert through the succinimide intermediate e.g., as illustrated below.

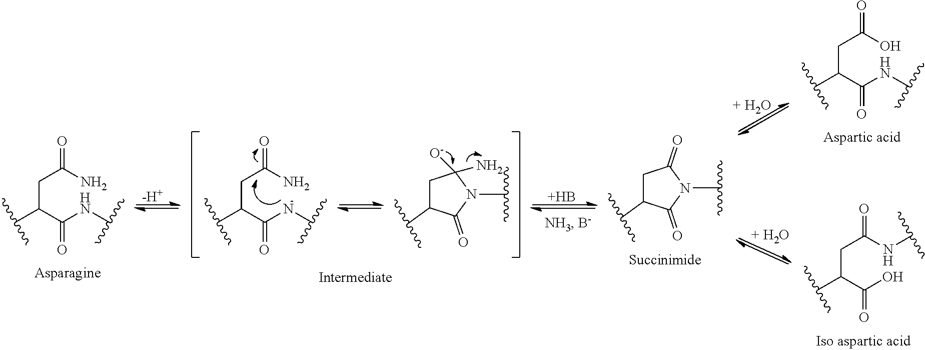

As provided herein, each deamidated N in the VP1, VP2 or VP3 may independently be aspartic acid (Asp), isoaspartic acid (isoAsp), aspartate, and/or an interconverting blend of Asp and isoAsp, or combinations thereof. Any suitable ratio of α- and isoaspartic acid may be present. For example, in certain embodiments, the ratio may be from 10:1 to 1:10 aspartic to isoaspartic, about 50:50 aspartic: isoaspartic, or about 1:3 aspartic: isoaspartic, or another selected ratio.

In certain embodiments, one or more glutamine (Q) may be derivatized (deamidate) to glutamic acid (Glu), i.e., α-glutamic acid, γ-glutamic acid (Glu), or a blend of α- and γ-glutamic acid, which may interconvert through a common glutarinimide intermediate. Any suitable ratio of α- and γ-glutamic acid may be present. For example, in certain embodiments, the ratio may be from 10:1 to 1:10 α to γ, about 50:50 α:γ, or about 1:3 α:γ or another selected ratio.

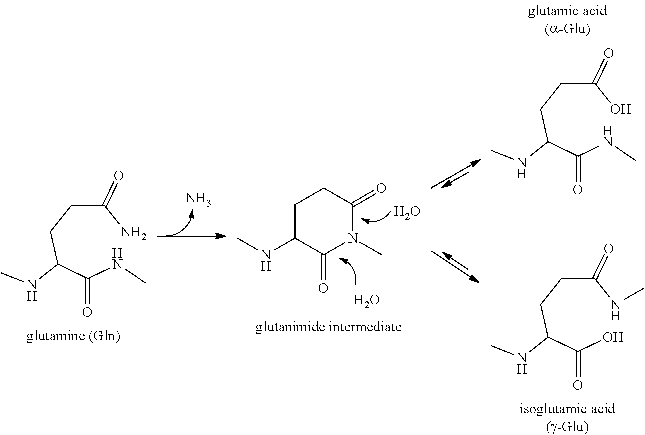

Thus, an rAAV includes subpopulations within the rAAV capsid of vp1, vp2 and/or vp3 proteins with deamidated amino acids, including at a minimum, at least one subpopulation comprising at least one highly deamidated asparagine. In addition, other modifications may include isomerization, particularly at selected aspartic acid (D or Asp) residue positions. In still other embodiments, modifications may include an amidation at an Asp position.

In certain embodiments, an AAV capsid contains subpopulations of vp1, vp2 and vp3 having at least 4 to at least about 25 deamidated amino acid residue positions, of which at least 1 to 10% are deamidated as compared to the encoded amino acid sequence of the vp proteins. The majority of these may be N residues. However, Q residues may also be deamidated.

In certain embodiments, a rAAV has an AAV capsid having vp1, vp2 and vp3 proteins having subpopulations comprising combinations of two, three, four or more deamidated residues at the positions set forth in the tables provided in the examples and incorporated herein by reference. Deamidation in the rAAV may be determined using 2D gel electrophoresis, and/or mass spectrometry, and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra. Examples of suitable proteases may include, e.g., trypsin or chymotrypsin. Mass spectrometric identification of deamidated peptides is relatively straightforward, as deamidation adds to the mass of intact molecule+0.984 Da (the mass difference between —OH and —NH₂ groups). The percent deamidation of a particular peptide is determined by the mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It will be understood by one of skill in the art that a number of variations on these illustrative methods can be used. For example, suitable mass spectrometers may include, e.g. a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

In addition to deamidations, other modifications may occur do not result in conversion of one amino acid to a different amino acid residue. Such modifications may include acetylated residues, isomerizations, phosphorylations, or oxidations.

Modulation of Deamidation: In certain embodiments, the AAV is modified to change the glycine in an asparagine-glycine pair, to reduce deamidation. In other embodiments, the asparagine is altered to a different amino acid, e.g., a glutamine which deamidates at a slower rate; or to an amino acid which lacks amide groups (e.g., glutamine and asparagine contain amide groups); and/or to an amino acid which lacks amine groups (e.g., lysine, arginine and histidine contain amine groups). As used herein, amino acids lacking amide or amine side groups refer to, e.g., glycine, alanine, valine, leucine, isoleucine, serine, threonine, cystine, phenylalanine, tyrosine, or tryptophan, and/or proline. Modifications such as described may be in one, two, or three of the asparagine-glycine pairs found in the encoded AAV amino acid sequence. In certain embodiments, such modifications are not made in all four of the asparagine-glycine pairs. A method for reducing deamidation of AAV and/or engineered AAV variants having lower deamidation rates is provided herein. Additionally, or alternative one or more other amide amino acids may be changed to a non-amide amino acid to reduce deamidation of the AAV. In certain embodiments, a mutant AAV capsid as described herein contains a mutation in an asparagine-glycine pair, such that the glycine is changed to an alanine or a serine. A mutant AAV capsid may contain one, two or three mutants where the reference AAV natively contains four NG pairs. In certain embodiments, an AAV capsid may contain one, two, three or four such mutants where the reference AAV natively contains five NG pairs. In certain embodiments, a mutant AAV capsid contains only a single mutation in an NG pair. In certain embodiments, a mutant AAV capsid contains mutations in two different NG pairs. In certain embodiments, a mutant AAV capsid contains mutation is two different NG pairs which are located in structurally separate location in the AAV capsid. In certain embodiments, the mutation is not in the VP1-unique region. In certain embodiments, one of the mutations is in the VP1-unique region. Optionally, a mutant AAV capsid contains no modifications in the NG pairs, but contains mutations to minimize or eliminate deamidation in one or more asparagines, or a glutamine, located outside of an NG pair.

In certain embodiments, a method of increasing the potency of a rAAV vector is provided which comprises engineering an AAV capsid which eliminating one or more of the NGs in the wild-type AAV capsid. In certain embodiments, the coding sequence for the “G” of the “NG” is engineered to encode another amino acid. In certain examples below, an “S” or an “A” is substituted. However, other suitable amino acid coding sequences may be selected. See, e.g., the tables below in which based on the numbering of AAV8, the coding sequence for at least one of the following positions: N57+1, N263+1, N385+1, N514+1, N540+1, is modified. In certain embodiments, AAV8 mutants avoid changing the NG pairs at positions N57, N94, N263, N305, Q467, N479, and/or N653. In certain embodiments, other AAVs avoid mutation at corresponding N positions as determined based on an alignment with AAV8, using AAV8 numbering as a reference.

These amino acid modifications may be made by conventional genetic engineering techniques. For example, a nucleic acid sequence containing modified AAV vp codons may be generated in which one to three of the codons encoding glycine in asparagine-glycine pairs are modified to encode an amino acid other than glycine. In certain embodiments, a nucleic acid sequence containing modified asparagine codons may be engineered at one to three of the asparagine-glycine pairs, such that the modified codon encodes an amino acid other than asparagine. Each modified codon may encode a different amino acid. Alternatively, one or more of the altered codons may encode the same amino acid. In certain embodiments, these modified AAV nucleic acid sequences may be used to generate a mutant rAAV having a capsid with lower deamidation than the native capsid. Such mutant rAAV may have reduced immunogenicity and/or increase stability on storage, particularly storage in suspension form.

Also provided herein are nucleic acid sequences encoding the AAV capsids having reduced deamidation. It is within the skill in the art to design nucleic acid sequences encoding this AAV capsid, including DNA (genomic or cDNA), or RNA (e.g., mRNA). Such nucleic acid sequences may be codon-optimized for expression in a selected system (i.e., cell type) can be designed by various methods. This optimization may be performed using methods which are available on-line (e.g., GeneArt), published methods, or a company which provides codon optimizing services, e.g., DNA2.0 (Menlo Park, Calif.). One codon optimizing method is described, e.g., in US International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence and produce a nucleic acid fragment of a codon-optimized coding region which encodes the polypeptide. A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In certain embodiments, AAV capsids are provided which have a heterogeneous population of AAV capsid isoforms (i.e., VP1, VP2, VP3) which contain multiple highly deamidated “NG” positions. In certain embodiments, the highly deamidated positions are in the locations identified below, with reference to the predicted full-length VP1 amino acid sequence. In other embodiments, the capsid gene is modified such that the referenced “NG” is ablated, and a mutant “NG” is engineered into another position.

In certain embodiments, AAV1 is characterized by a capsid composition of a heterogeneous population of VP isoforms which are deamidated as defined in the following table, based on the total amount of VP proteins in the capsid, as determined using mass spectrometry.

In certain embodiments, the AAV capsid is modified at one or more of the following positions, in the ranges provided below, as determined using mass spectrometry. Suitable modifications include those described in the paragraph above labelled modulation of deamidation, which is incorporated herein.

In certain embodiments, one or more of the following positions, or the glycine following the N is modified as described herein. In certain embodiments, an AAV1 mutant is constructed in which the glycine following the N at position 57, 383, 512 and/or 718 are preserved (i.e., remain unmodified). In certain embodiments, the NG at the four positions identified in the preceding sentence are preserved with the native sequence. Residue numbers are based on the published AAV1 VP1, reproduced in SEQ ID NO: 1.

In certain embodiments, an artificial NG is introduced into a different position than one of the positions identified below.

Residue numbers are based on the published AAV1 sequence, reproduced in SEQ ID NO: 1.

TABLE A

| AAV1 Capsid | |
|---|---|
| Position Based on | |
| VP1 numbering | % |
| N35 + Deamidation | 1-15, 5-10 |
| ~N57 + Deamidation | 65-90, 70-95, 80-95, 75-100, |
| | 80-100, or 90-100 |
| N113 + Deamidation | 0-8 |
| ~N223 + Deamidation | 0-30, 0, 20-28 |
| N227 + Deamidation | 0, 1-5 |
| ~N253 + Deamidation | 0, 1-35 |
| Q259 + Deamidation | 0, 10-25 |
| ~N269 + Deamidation | 0-25 |
| ~N271 + Deamidation | 0-25 |
| N286 + Deamidation | 2-10 |
| ~N302 + Deamidation | 10-50 |
| ~N303 + Deamidation | 0-55 |
| ~N383 + Deamidation | 65-90, 70-95, 80-95, 75-100, |
| | 80-100, or 90-100 |
| ~N408 + Deamidation | 30-65 |
| ~N451 + Deamidation | 0-25 |
| ~Q452 + Deamidation | 0-5 |
| N477 + Deamidation | 0-45 |
| ~N496 + Deamidation | 0-75 |
| N512 + Deamidation | 75-100, 80-100, 90-100 |
| N651 + Deamidation | 0-3 |
| N691 + Deamidation | 0, 1-60 |
| ~N704 + Deamidation | 0-10 |
| N718 + Deamidation | 75-100, 80-100, 90-100 |

In certain embodiments, an AAV3B capsid characterized by a capsid composition of a heterogeneous population of VP isoforms which are deamidated as defined in the following table, based on the total amount of VP proteins in the capsid, as determined using mass spectrometry. In certain embodiments, the AAV capsid is modified at one or more of the following position, in the ranges provided below, as determined using mass spectrometry. Suitable modifications include those described in the paragraph above labelled modulation of deamidation, which is incorporated herein. In certain embodiments, one or more of the following positions, or the glycine following the N is modified as described herein. In certain embodiments, an AAV3 mutant is constructed in which the glycine following the N at position 57, 383, 512 and/or 718 are preserved (i.e., remain unmodified). In certain embodiments, the NG at the four positions identified in the preceding sentence are preserved with the native sequence. Residue numbers are based on the published AAV3B VP1, reproduced in SEQ ID NO: 2. In certain embodiments, an artificial NG is introduced into a different position than one of the positions identified below. In certain embodiments, the capsid is modified to reduce “N” or “Q” at positions other than then “NG” pairs. Residue numbers are based on the published AAV3B sequence, reproduced in SEQ ID NO: 2.

TABLE B

| AAV3B Capsid | |
|---|---|
| Position based on | |
| VP1 numbering | % |
| ~N57 + Deamidation | 65-90, 70-95, 80-95, 75-100 |
| | 100, 80-100, or 90-100 |
| ~N223 + Deamidation | 10-30, 15-25 |
| N227 + Deamidation | 0-5 |
| ~N253 + Deamidation | 1-10, 2-8 |
| ~Q259 + Deamidation | 0-3 |
| ~N302 + Deamidation | 1-30, 5- 25, 10-25 |
| | 65-90, 70-95, 80-95, 75-100, |
| ~N382 + Deamidation | 80-100, or 90-100 |
| N477 + Deamidation | 0-1 |
| | 65-90, 70-95, 80-95, 75-100, |
| ~N512 + Deamidation | 80-100, |
| | or 90-100 |
| ~N582 + Deamidation | 1-20, 5-15 |
| ~Q599 + Deamidation | 0-5 |
| N691 + Deamidation | 5-20 |
| N718 + Deamidation | 75 - 100 |

In certain embodiments, an AAV5 capsid characterized by a capsid composition of a heterogeneous population of VP isoforms which are deamidated as defined in the following table, based on the total amount of VP proteins in the capsid, as determined using mass spectrometry. In certain embodiments, the AAV capsid is modified at one or more of the following position, in the ranges provided below, as determined using mass spectrometry. Suitable modifications include those described in the paragraph above labelled modulation of deamidation, which is incorporated herein. In certain embodiments, one or more of the following positions, or the glycine following the N is modified as described herein. In certain embodiments, an artificial NG is introduced into a different position than one of the positions identified below. In certain embodiments, the capsid is modified to reduce “N” or “Q” at positions other than then “NG” pairs. Residue numbers are based on the published AAV5 sequence, reproduced in SEQ ID NO: 3.

TABLE C

| AAV5 Capsid | |
|---|---|
| Position based on | |
| VP1 numbering | % |
| N34 + Deamidation | 1-15, 2-10 |
| N56 + Deamidation | 65-90, 70-95, 80-95, 75-100, |
| | 80-100, or 90-100 |
| N112 +Deamidation | 1-5 |
| ~N213 + Deamidation | 5 - 25, 15-25 |
| ~N243 + Deamidation | 15- 45, 30-35 |
| ~N292 + Deamidation | 15-40, 20-30 |
| N325+Deamidation | 5-15 |
| N347 + Deamidation | 65-90, 70-95, 80-95, 75-100, |
| | 80-100, or 90-100 |
| ~N400 + Deamidation | 1-10 |
| ~Q421 + Deamidation | 1-10 |
| ~N442 + Deamidation | 5-30, 10-30 |
| ~N459 + Deamidation | 5-20, 10-15 |
| | 65-90, 70-95, 80-95, 75-100 |
| ~N509 + Deamidation | 100, 80-100, or 90-100 |
| ~N691 + Deamidation | 10-40, 15-30 |

In certain embodiments, an AAV7 capsid is characterized by a capsid composition of a heterogeneous population of VP isoforms which are deamidated as defined in the following table, based on the total amount of VP proteins in the capsid, as determined using mass spectrometry. In certain embodiments, the AAV capsid is modified at one or more of the following position, in the ranges provided below, as determined using mass spectrometry. Suitable modifications include those described in the paragraph above labelled modulation of deamidation, which is incorporated herein. In certain embodiments, one or more of the following positions, or the glycine following the N is modified as described herein. In certain embodiments, an artificial NG is introduced into a different position than one of the positions identified below. In certain embodiments, the capsid is modified to reduce “N” or “Q” at positions other than then “NG” pairs. Residue numbers are based on the published AAV7 sequence, reproduced in SEQ ID NO: 4.

TABLE D

| AAV7 Capsid Position | |
|---|---|
| based on VP1 | |
| numbering | % |
| N41 + Deamidation | 65-90, 70-95, 80-95, 75- |
| | 100, 80-100, or 90-100 |
| ~N57 + Deamidation | 65-90, 70-95, 80-95, 75- |
| | 100, 80-100, or 90-100 |
| N66 + Deamidation | 5-25, 10-20 |
| ~N224 + Deamidation | 5-20, 5-15 |
| N228 + Deamidation | 0-5 |
| ~N304 + Deamidation | 15-35, 20-30 |
| ~N384 + Deamidation | 65-90, 70-95, 80-95, 75- |
| | 100, 80-100, or 90-100 |
| N479 + Deamidation | 0-5 |
| ~N499 + Deamidation | 1-30, 5-25 |
| N514 + Deamidation | 65-90, 70-95, 80-95, 75- |
| | 100, 80-100, or 90-100 |
| ~N517 + Deamidation | 1-15, 5-15 |
| N705 + Deamidation | 1-15, 5-15 |
| N736 + Deamidation | 1-20, 5-20 |

In certain embodiments, an AAVrh32.33 capsid characterized by a capsid composition of a heterogeneous population of VP isoforms which are deamidated as defined in the following table, based on the total amount of VP proteins in the capsid, as determined using mass spectrometry. In certain embodiments, the AAV capsid is modified at one or more of the following position, in the ranges provided below, as determined using mass spectrometry. Suitable modifications include those described in the paragraph above labelled modulation of deamidation, which is incorporated herein. In certain embodiments, one or more of the following positions, or the glycine following the N is modified as described herein. In certain embodiments, an artificial NG is introduced into a different position than one of the positions identified below. In certain embodiments, the capsid is modified to reduce “N” or “Q” at positions other than then “NG” pairs. Residue numbers are based on the published AAVrh32.33 sequence, reproduced in SEQ ID NO: 5.

TABLE E

| AAVrh32.33 Position | |
|---|---|
| Based on VP1 | |
| numbering | Avg % |
| N14 + Deamidation | 1-10 |
| N57 + Deamidation | 50-100 |
| N113 + Deamidation | 0-3 |
| Q210 + Deamidation | 0-20, 5-20, 10-20 |
| N247 + Deamidation | 10-40, 20-35, 25-35 |
| ~N264 + Deamidation | 50-100 |
| ~N292 + Deamidation | 25-75, 30-60, 40-55 |
| Q310 + Deamidation | 1-8 |
| ~N318 + Deamidation | 65-90, 70-95, 80-95, 75- |
| | 100, 80-100, or 90-100 |
| N383 + Deamidation | 0-5 |
| ~N400 + Deamidation | 10-40, 20-40, 30-40 |
| ~Q449 + Deamidation | 0-5 |
| N470 + Deamidation | 0-5 |
| N498 + Deamidation | 0-1 |

In certain embodiments, an AAV8 capsid is characterized by a capsid composition of a heterogeneous population of VP isoforms which are deamidated as defined in the following table, based on the total amount of VP proteins in the capsid, as determined using mass spectrometry. Suitable modifications include those described in the paragraph above labelled modulation of deamidation, which is incorporated herein. In certain embodiments, the AAV capsid is modified at one or more of the following position, in the ranges provided below, as determined using mass spectrometry. In certain embodiments, one or more of the following positions, or the glycine following the N is modified as described herein. In certain embodiments, an artificial NG is introduced into a different position than one of the positions identified below. In certain embodiments, an artificial NG is introduced into a different position than one of the positions identified below. In certain embodiments, one or more of the following positions, or the glycine following the N is modified as described herein. For example, in certain embodiments, a G may be modified to an S or an A, e.g., at position 58, 67, 95, 216, 264, 386, 411, 460, 500, 515, or 541. Significant reduction in deamidation is observed when NG57/58 is altered to NS 57/58 or NA57/58. However, in certain embodiments, an increase in deamidation is observed when NG is altered to NS or NA. In certain embodiments, an N of an NG pair is modified to a Q while retaining the G. In certain embodiments, both amino acids of an NG pair are modified. In certain embodiments, N385Q results in significant reduction of deamidation in that location. In certain embodiments, N499Q results in significant increase of deamidation in that location. In certain embodiments, an NG mutation is made at the pair located at N263 (e.g., to N263A). In certain embodiments, an NG mutation is made at the pair located at N514 (e.g., to N514A). In certain embodiments, an NG mutation is made at the pair located at N540 (e.g., N540A). In certain embodiments, AAV mutants containing multiple mutations and at least one of the mutations at these position are engineered. In certain embodiments, no mutation is made at position N57. In certain embodiments, no mutation is made at position N94. In certain embodiments, no mutation is made at position N305. In certain embodiments, no mutation is made at position G386. In certain embodiments, no mutation is made at position Q467. In certain embodiments, no mutation is made at position N479. In certain embodiments, no mutation is made at position N653. In certain embodiments, the capsid is modified to reduce “N” or “Q” at positions other than then “NG” pairs. Residue numbers are based on the published AAV8 sequence, reproduced in SEQ ID NO: 6.

TABLE F

| AAV8 Modification | |
|---|---|
| Based on VP1 numbering | % |
| N35 + Deamidation | 1 |
| N57 + Deamidation | 65-90, 70-95, 80-95, 75- |
| | 100, 80-100, or 90-100 |
| N66 + Deamidation | 0-10 |
| N94 + Deamidation | 1-15 |
| N113 + Deamidation | 0-10 |
| ~Q166 + Deamidation | 0-10 |
| ~N173 + Deamidation | 0-10 |
| N254/N255 + Deamidation | 5-45 |
| N263 + Deamidation | 65-90, 70-95, 80-95, 75- |
| | 100, 80-100, or 90-100 |
| ~N304 + Deamidation | 0-10 |
| ~N305 + Deamidation | 10-40 |
| N320 + Deamidation | 0-10 |
| ~Q322 + Deamidation | 0-10 |
| N385 + Deamidation | 65-90, 70-95, 80-95, 75- |
| | 100, 80-100, or 90-100 |
| N410 + Deamidation | 15-70 |
| ~Q431 + Deamidation | 0-10 |
| N438 + Deamidation | 0-10 |
| ~N459 + Deamidation | 0-10 |
| ~Q467 + Deamidation | 0-10 |
| ~N479 + Deamidation | 0-10 |
| N498/N499 + Deamidation | 0-10 |
| N502 + Deamidation | 0-10 |
| N514 + Deamidation | 65-90, 70-95, 80-95, 75- |
| | 100, 80-100, or 90-100 |
| N517 + Deamidation | 15-40 |
| N540 + Deamidation | 65-90, 70-95, 80-95, 75- |
| | 100, 80-100, or 90-100 |
| ~N554 + Deamidation | 0-10 |
| ~Q589 + Deamidation | 0-10 |
| ~N590 + Deamidation | 0-10 |
| ~N599 + Deamidation | 35-75 |
| ~Q601 + Deamidation | 45-75 |
| ~Q610 + Deamidation | 0-10 |
| Q617 + Deamidation | 0-10 |
| N630 + Deamidation | 5-30 |
| Q648 + Deamidation | 0-10 |
| N653 + Deamidation | 0-10 |
| N665 + Deamidation | 5-30 |
| N670 + Deamidation | 0-10 |
| N693 + Deamidation | 0-10 |
| ~N706 + Deamidation | 0-10 |
| N718 + Deamidation | 0-10 |
| N737 + Deamidation | 0-10 |

In certain embodiments, mutants may include AAV8 G264A/G515A (SEQ ID NO: 21), AAV8G264A/G541A (SEQ ID NO: 23), AAV8G515A/G541A (SEQ ID NO: 25), or AAV8 G264A/G515A/G541A (SEQ ID NO: 27). In certain embodiments, nucleic acid sequences encoding these mutant AAV8 capsids are provided. In certain embodiments, the nucleic acid sequences are provided in, e.g., SEQ ID NO: 20 (AAV8 G264A/G515A), SEQ ID NO: 22 (AAV8G264A/G541A), SEQ ID NO: 24 (AAV8G515A/G541A), or SEQ ID NO: 26 (AAV8 G264A/G515A/G541A). In certain embodiments, an AAV8 mutant may be N499Q, N459Q, N305Q/N459Q, N305QN499Q, N459Q, N305Q/N459Q, N305q/N499Q, or N205Q, N459Q, or N305Q/N459Q, N499Q. In certain embodiments, these mutations are combined with a G264A/G541A mutation. In certain embodiments, the mutation is AAV8 G264A/G541A/N499Q (SEQ ID NO: 115); AAV8 G264A/G541A/N459Q (SEQ ID NO: 116); AAV8 G264A/G541A/N305Q/N459Q (SEQ ID NO: 117); AAV8 G264A/G541A/N305Q/N499Q (SEQ ID NO: 118); G264A/G541A/N459Q/N499Q (SEQ ID NO: 119); or AAV8 G264A/G541A/N305Q/N459Q/N499Q (SEQ ID NO: 120). Also encompassed are nucleic acid sequences encoding these AAV8 mutants.

In certain embodiments, an AAV9 capsid is characterized by a capsid composition of a heterogeneous population of VP isoforms which are deamidated as defined in the following table, based on the total amount of VP proteins in the capsid, as determined using mass spectrometry. In certain embodiments, the AAV capsid is modified at one or more of the following position, in the ranges provided below, as determined using mass spectrometry. Suitable modifications include those described in the paragraph above labelled modulation of deamidation, which is incorporated herein. In certain embodiments, one or more of the following positions, or the glycine following the N is modified as described herein. In certain embodiments, the AAV9 capsid encoding position N214/G215 is modified to N214Q, which is observed to have significantly increased deamidation. In certain embodiments, an NG mutation is made at the pair located at N452 (e.g., to N452A). In certain embodiments, no mutation is made at position N57. In certain embodiments, AAV mutants containing multiple mutations and at least one of the mutations at these position are engineered. In certain embodiments, an artificial NG is introduced into a different position than one of the positions identified below. In certain embodiments, the capsid is modified to reduce “N” or “Q” at positions other than then “NG” pairs. Residue numbers are based on the published AAV9 sequence, reproduced in SEQ ID NO: 7.

TABLE G

| AAV9 | |
|---|---|
| Modifications based | |
| on VP1 numbering | % |
| N57 + Deamidation | 65-90, 70-95, 80-95, 75- |
| | 100, 80-100, or 90-100 |
| N94 + Deamidation | 1-10, 2-8 |
| N113 + Deamidation | 0-10 |
| ~N214 + Deamidation | 0-10 |
| N227 + Deamidation | 0-10 |
| N253 + Deamidation | 5-15 |
| N254 + Deamidation | 1-5 |
| Q259 + Deamidation | 0-10 |
| N270 + Deamidation | 5-20, 5-15 |
| N304 + Deamidation | 10-30, 15-30 |
| N314 + Deamidation | 0-10 |
| N319 + Deamidation | 0-10 |
| Q321 + Deamidation | 0-1- |
| N329 + Deamidation | 65-90, 70-95, 80-95, 75- |
| | 100, 80-100, or 90-100 |
| N383 + Deamidation | |
| N409 + Deamidation | 5-20, 5-15 |
| N437 + Deamidation | |
| N452 + Deamidation | |
| N470 + Deamidation | |
| N477 + Deamidation | 1-5 |
| ~N497 + Deamidation | |
| N512 + Deamidation | 65-90, 70-95, 80-95, 75- |
| | 100, 80-100, or 90-100 |
| N515 + Deamidation | 1-5 |
| N519 + Deamidation | 1-5 |
| N628 + Deamidation | |
| N651 + Deamidation | 1-3 |
| N663 + Deamidation | 1-10, 2-8 |
| ~N668 + Deamidation | 5-20 |
| ~N704 + Deamidation | 1-10 |
| N709 + Deamidation | 1-10 |

Additionally, or alternatively, an AAVhu37 capsid comprises: a heterogeneous population of vp11 proteins which are the product of a nucleic acid sequence encoding the amino acid sequence of SEQ ID NO: 36, a heterogeneous population of vp2 proteins which are the product of a nucleic acid sequence encoding the amino acid sequence of at least about amino acids 138 to 738 of SEQ ID NO: 36, and a heterogeneous population of vp3 proteins which are the product of a nucleic acid sequence encoding at least amino acids 204 to 738 of SEQ ID NO: 36 wherein: the vp, vp2 and vp3 proteins contain subpopulations with amino acid modifications comprising at least two highly deamidated asparagnes (N) in asparagine-glycine pairs in SEQ ID NO: 36 and optionally further comprising subpopulations comprising other deamidated amino acids, wherein the deamidation results in an amino acid change. AAVhu37 is characterized by having highly deamidated residues, e.g., at positions N57, N263, N385, and/or N514 based on the numbering of the AAVhu37 VP1 (SEQ ID NO: 36).

Deamidation has been observed in other residues, as shown in the table below and in the examples. In certain embodiments, an AAVhu37 capsid is modified in one or more of the following positions, in the ranges provided below, as determined using mass spectrometry with a trypsin enzyme. In certain embodiments, one or more of the following positions, or the glycine following the N is modified as described herein. For example, in certain embodiments, a G may be modified to an S or an A, e.g., at position 58, 264, 386, or 515. In one embodiment, the AAVhu37 capsid is modified at position N57/G58 to N57Q or G58A to afford a capsid with reduced deamidation at this position. In another embodiment, N57/G58 is altered to NS57/58 or NA57/58. However, in certain embodiments, an increase in deamidation is observed when NG is altered to NS or NA. In certain embodiments, an N of an NG pair is modified to a Q while retaining the G. In certain embodiments, both amino acids of an NG pair are modified. In certain embodiments, N385Q results in significant reduction of deamidation in that location. In certain embodiments, N499Q results in significant increase of deamidation in that location.

In certain embodiments, AAVhu37 may have these or other residues deamidated, e.g., typically at less than 10% and/or may have other modifications, including methylations (e.g, ˜R487) (typically less than 5%, more typically less than 1% at a given residue), isomerization (e.g., at D97) (typically less than 5%, more typically less than 1% at a given residue, phosphorylation (e.g., where present, in the range of about 10 to about 60%, or about 10 to about 30%, or about 20 to about 60%) (e.g., at one or more of S149, ˜S153, ˜S474, ˜T570, ˜S665), or oxidation (e.g, at one or more of W248, W307, W307, M405, M437, M473, W480, W480, W505, M526, M544, M561, W621, M637, and/or W697). Optionally the W may oxidize to kynurenine.

TABLE H

| AAVhu37 | |
|---|---|
| Deamidation | |
| based on VP1 | |
| numbering | % Deamidation |
| N57 + Deamidation | 65-90, 70-95, 80- |
| | 95, 75-100, 80- |
| | 100, or 90-100 |
| N94 + Deamidation | 5-15, about 10 |
| ~N254 + Deamidation | 10-20 |
| ~N263 + Deamidation | 75-100 |
| ~N305 + Deamidation | 1-5 |
| ~N385 + Deamidation | 65-90, 70-95, 80- |
| | 95, 75-100, 80- |
| | 100, or 90-100 |
| ~N410 + Deamidation | 1-25, |
| N479 + Deamidation | 1-5, 1-3 |
| ~N514 + Deamidation | 65-90, 70-95, 80- |
| | 95, 75-100, 80- |
| | 100, or 90-100 |
| ~Q601 + Deamidation | 0-1 |
| N653 + Deamidation | 0-2 |

Still other positions may have such these or other modifications (e.g., acetylation or further deamidations). In certain embodiments, the nucleic acid sequence encoding the AAVhu37 vp1 capsid protein is provided in SEQ ID NO: 37. In other embodiments, a nucleic acid sequence of 70% to 99.9% identity to SEQ ID NO: 37 may be selected to express the AAVhu37 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 37. However, other nucleic acid sequences which encode the amino acid sequence of SEQ ID NO: 36 may be selected for use in producing rAAVhu37 capsids. In certain embodiments, the nucleic acid sequence has the nucleic acid sequence of SEQ ID NO: 37 or a sequence at least 70% to 99.% identical, at least 75%, at least 80%, at least 85%, at least 90%, at least 95%, at least 97%, at least 99%, identical to SEQ ID NO: 37 which encodes SEQ ID NO: 36. In certain embodiments, the nucleic acid sequence has the nucleic acid sequence of SEQ ID NO: 37 or a sequence at least 70% to 99.%, at least 75%, at least 80%, at least 85%, at least 90%, at least 95%, at least 97%, at least 99%, identical to about nt 412 to about nt 2214 of SEQ ID NO: 37 which encodes the vp2 capsid protein (about aa 138 to 738) of SEQ ID NO: 36. In certain embodiments, the nucleic acid sequence has the nucleic acid sequence of about nt 610 to about nt 2214 of SEQ ID NO: 37 or a sequence at least 70% to 99.%, at least 75%, at least 80%, at least 85%, at least 90%, at least 95%, at least 97%, at least 99%, identical to nt SEQ ID NO: 37 which encodes the vp3 capsid protein (about aa 204 to 738) of SEQ ID NO: 36. See, EP 2 345 731 B1 and SEQ ID NO: 88 therein, which are incorporated by reference.

As used herein, “encoded amino acid sequence” refers to the amino acid which is predicted based on the translation of a known DNA codon of a referenced nucleic acid sequence being translated to an amino acid. The following table illustrates DNA codons and twenty common amino acids, showing both the single letter code (SLC) and three letter code (3LC).

| Amino Acid | SLC | 3 LC | DNA codons |
|---|---|---|---|
| Isoleucine | I | Ile | ATT, ATC, ATA |
| Leucine | L | Leu | CTT, CTC, CTA, CTG, TTA, TTG |
| Valine | V | Val | GTT, GTC, GTA, GTG |
| Phenylalanine | F | Phe | TTT, TTC |
| Methionine | M | Met | ATG |
| Cysteine | C | Cys | TGT, TGC |
| Alanine | A | Ala | GCT, GCC, GCA, GCG |
| Glycine | G | Gly | GGT, GGC, GGA, GGG |
| Proline | P | Pro | CCT, CCC, CCA, CCG |
| Threonine | T | Thr | ACT, ACC, ACA, ACG |
| Serine | S | Ser | TCT, TCC, TCA, TCG, AGT, AGC |
| Tyrosine | Y | Tyr | TAT, TAC |
| Tryptophan | W | Trp | TGG |
| Glutamine | Q | Gln | CAA, CAG |
| Asparagine | N | Asn | AAT, AAC |
| Histidine | H | His | CAT, CAC |
| Glutamic acid | E | Glu | GAA, GAG |
| Aspartic acid | D | Asp | GAT, GAC |
| Lysine | K | Lys | AAA, AAG |
| Arginine | R | Arg | CGT, CGC, CGA, CGG, AGA, AGG |
| Stop codons | Stop | | TAA, TAG, TGA |

rAAV Vectors

As indicated above, the novel AAV sequences and proteins are useful in production of rAAV, and are also useful in recombinant AAV vectors which may be antisense delivery vectors, gene therapy vectors, or vaccine vectors. Additionally, the engineered AAV capsids described herein may be used to engineer rAAV vectors for delivery of a number of suitable nucleic acid molecules to target cells and tissues.

Genomic sequences which are packaged into an AAV capsid and delivered to a host cell are typically composed of, at a minimum, a transgene and its regulatory sequences, and AAV inverted terminal repeats (ITRs). Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences from AAV2. A shortened version of the 5′ ITR, termed AITR, has been described in which the D-sequence and terminal resolution site (trs) are deleted. In other embodiments, the full-length AAV 5′ and 3′ ITRs are used. However, ITRs from other AAV sources may be selected. Where the source of the ITRs is from AAV2 and the AAV capsid is from another AAV source, the resulting vector may be termed pseudotyped. However, other configurations of these elements may be suitable.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

The regulatory control elements typically contain a promoter sequence as part of the expression control sequences, e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be used may be utilized in the vectors described herein. The promoter(s) can be selected from different sources, e.g., human cytomegalovirus (CMV) immediate-early enhancer/promoter, the SV40 early enhancer/promoter, the JC polyomavirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter. In addition to a promoter a vector may contain one or more other appropriate transcription initiation, termination, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. An example of a suitable enhancer is the CMV enhancer. Other suitable enhancers include those that are appropriate for desired target tissue indications. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g, the chicken beta-actin intron. Other suitable introns include those known in the art, e.g., such as are described in WO 2011/126808. Examples of suitable polyA sequences include, e.g., SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence [see, e.g., MA Zanta-Boussif, et al, Gene Therapy (2009) 16: 605-619.

These rAAVs are particularly well suited to gene delivery for therapeutic purposes and for immunization, including inducing protective immunity. Further, the compositions of the invention may also be used for production of a desired gene product in vitro. For in vitro production, a desired product (e.g., a protein) may be obtained from a desired culture following transfection of host cells with a rAAV containing the molecule encoding the desired product and culturing the cell culture under conditions which permit expression. The expressed product may then be purified and isolated, as desired. Suitable techniques for transfection, cell culturing, purification, and isolation are known to those of skill in the art.

### Therapeutic Transgenes

Useful products encoded by the transgene include a variety of gene products which replace a defective or deficient gene, inactivate or “knock-out”, or “knock-down” or reduce the expression of a gene which is expressing at an undesirably high level, or delivering a gene product which has a desired therapeutic effect. In most embodiments, the therapy will be “somatic gene therapy”, i.e., transfer of genes to a cell of the body which does not produce sperm or eggs. In certain embodiments, the transgenes express proteins have the sequence of native human sequences. However, in other embodiments, synthetic proteins are expressed. Such proteins may be intended for treatment of humans, or in other embodiments, designed for treatment of animals, including companion animals such as canine or feline populations, or for treatment of livestock or other animals which come into contact with human populations.

Examples of suitable gene products may include those associated with familial hypercholesterolemia, muscular dystrophy, cystic fibrosis, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB—P51608), Amyotrophic Lateral Sclerosis (ALS), Duchenne Type Muscular dystrophy, Friedrichs Ataxia (e.g., frataxin), progranulin (PRGN) (associated with non-Alzheimer's cerebral degenerations, including, frontotemporal dementia (FTD), progressive non-fluent aphasia (PNFA) and semantic demential), among others. See, e.g., www.orpha.net/consor/cgi-bin/Disease_Search_List.php; rarediseases.info.nih.gov/diseases.

Examples of suitable genes may include, e.g., hormones and growth and differentiation factors including, without limitation, insulin, glucagon, glucagon-like peptide-1 (GLP1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO) (including, e.g., human, canine or feline epo), connective tissue growth factor (CTGF), neutrophic factors including, e.g., basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-36 (including, e.g., human interleukins IL-1, IL-1a, IL-1, IL-2, IL-3, IL-4, IL-6, IL-8, IL-12, IL-11, IL-12, IL-13, IL-18, IL-31, IL-35), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. For example, in certain embodiments, the rAAV antibodies may be designed to delivery canine or feline antibodies, e.g., such as anti-IgE, anti-IL31, anti-CD20, anti-NGF, anti-GnRH. Useful gene products also include complement regulatory proteins such as complement regulatory proteins, membrane cofactor protein (MCP), decay accelerating factor (DAF), CR1, CF2, CD59, and C1 esterase inhibitor (C1-INH).

Still other useful gene products include any one of the receptors for the hormones, growth factors, cytokines, lymphokines, regulatory proteins and immune system proteins. The invention encompasses receptors for cholesterol regulation and/or lipid modulation, including the low density lipoprotein (LDL) receptor, high density lipoprotein (HDL) receptor, the very low density lipoprotein (VLDL) receptor, and scavenger receptors. The invention also encompasses gene products such as members of the steroid hormone receptor superfamily including glucocorticoid receptors and estrogen receptors, Vitamin D receptors and other nuclear receptors. In addition, useful gene products include transcription factors such asjun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase (OTC), arginosuccinate synthetase, arginosuccinate lyase (ASL) for treatment of arginosuccinate lyase deficiency, arginase, fumarylacetate hydrolase, phenylalanine hydroxylase, alpha-1 antitrypsin, rhesus alpha-fetoprotein (AFP), rhesus chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)).

In certain embodiments, the rAAV may be used in gene editing systems, which system may involve one rAAV or co-administration of multiple rAAV stocks. For example, the rAAV may be engineered to deliver SpCas9, SaCas9, ARCUS, Cpf1, and other suitable gene editing constructs.

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349). In some embodiments, the minigene comprises first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single minigene separated by 42 nucleic acids coding for 14 amino acids of the B domain [U.S. Pat. No. 6,200,560].

Other useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce “self”-directed antibodies. T cell mediated autoimmune diseases include Rheumatoid arthritis (RA), multiple sclerosis (MS), Sjbgren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Further illustrative genes which may be delivered via the rAAV include, without limitation, glucose-6-phosphatase, associated with glycogen storage disease or deficiency type 1A (GSD1), phosphoenolpyruate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-phosphate uridyl transferase, associated with galactosemia; phenylalanine hydroxylase, associated with phenylketonuria (PKU): branched chain alpha-ketoacid dehydrogenase, associated with Maple syrup urine disease: fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); Niemann-Pick disease, type C1); propionic academia (PA); low density lipoprotein receptor (LDLR) protein, associated with familial hypercholesterolemia (FH); UDP-glucouronosyltransferase, associated with Crigler-Najjar disease; adenosine deaminase, associated with severe combined immunodeficiency disease: hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccsinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; a-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

In certain embodiments, the rAAV described herein may be used in treatment of mucopolysaccaridoses (MPS) disorders. Such rAAV may contain carry a nucleic acid sequence encoding α-L-iduronidase (IDUA) for treating MPS I (Hurler, Hurler-Scheie and Scheie syndromes); a nucleic acid sequence encoding iduronate-2-sulfatase (IDS) for treating MPS II (Hunter syndrome); a nucleic acid sequence encoding sulfamidase (SGSH) for treating MPSIII A, B, C, and D (Sanfilippo syndrome); a nucleic acid sequence encoding N-acetylgalactosamine-6-sulfate sulfatase (GALNS) for treating MPS IV A and B (Morquio syndrome); a nucleic acid sequence encoding arylsulfatase B (ARSB) for treating MPS VI (Maroteaux-Lamy syndrome); a nucleic acid sequence encoding hyaluronidase for treating MPSI IX (hyaluronidase deficiency) and a nucleic acid sequence encoding beta-glucuronidase for treating MPS VII (Sly syndrome).

### Immunogenic Transgenes

In some embodiments, an rAAV vector comprising a nucleic acid encoding a gene product associated with cancer (e.g., tumor suppressors) may be used to treat the cancer, by administering a rAAV harboring the rAAV vector to a subject having the cancer. In some embodiments, an rAAV vector comprising a nucleic acid encoding a small interfering nucleic acid (e.g., shRNAs, miRNAs) that inhibits the expression of a gene product associated with cancer (e.g., oncogenes) may be used to treat the cancer, by administering a rAAV harboring the rAAV vector to a subject having the cancer. In some embodiments, an rAAV vector comprising a nucleic acid encoding a gene product associated with cancer (or a functional RNA that inhibits the expression of a gene associated with cancer) may be used for research purposes, e.g., to study the cancer or to identify therapeutics that treat the cancer. The following is a non-limiting list of exemplary genes known to be associated with the development of cancer (e.g., oncogenes and tumor suppressors): AARS, ABCB1, ABCC4, ABI2, ABL1, ABL2, ACK1, ACP2, ACY1, ADSL, AK1, AKR C2, AKT1, ALB, ANPEP, ANXA5, ANXA7, AP2M1, APC, ARHGAP5, ARHGEF5, ARID4A, ASNS, ATF4, ATM, ATP5B, ATP50, AXL, BARD1, BAX, BCL2, BHLHB2, BLMH, BRAF, BRCA1, BRCA2, BTK, CANX, CAP1, CAPN1, CAPNS1, CAV1, CBFB, CBLB, CCL2, CCND1, CCND2, CCND3, CCNE1, CCT5, CCYR61, CD24, CD44, CD59, CDCL2, CDCL5, CDCL5A, CDCL5B, CDCl₂L5, CDK10, CDK4, CDK5, CDK9, CDKL1, CDKN1A, CDKN1B, CDKN1C, CDKN2A, CDKN2B, CDKN2D, CEBPG, CENPC1, CGRRF1, CHAF1A, CIB1, CKMT1, CLK1, CLK2, CLK3, CLNS1A, CLTC, COL1A1, COL6A3, COX6C, COX7A2, CRAT, CRHR1, CSF1R, CSK, CSNK1G2, CTNNA1, CTNNB1, CTPS, CTSC, CTSD, CUL1, CYR61, DCC, DCN, DDX10, DEK, DHCR7, DHRS2, DHX8, DLG3, DVL1, DVL3, E2F1, E2F3, E2F5, EGFR, EGR1, EIF5, EPHA2, ERBB2, ERBB3, ERBB4, ERCC3, ETV1, ETV3, ETV6, F2R, FASTK, FBN1, FBN2, FES, FGFR1, FGR, FKBP8, FN1, FOS, FOSL1, FOSL2, FOXG1A, FOXO1A, FRAP1, FRZB, FTL, FZD2, FZD5, FZD9, G22P1, GAS6, GCN5L2, GDF15, GNA13, GNAS, GNB2, GNB2L1, GPR39, GRB2, GSK3A, GSPT1, GTF2I, HDAC1, HDGF, HMMR, HPRT1, HRB, HSPA4, HSPA5, HSPA8, HSPB1, HSPH1, HYAL 1, HYOUl, ICAM1, ID1, ID2, IDUA, IER3, IFITM1, IGF1R, IGF2R, IGFBP3, IGFBP4, IGFBP5, IL1B, ILK, ING1, IRF3, ITGA3, ITGA6, ITGB4, JAK1, JARID1A, JUN, JUNB, JUND, K-ALPHA-1, KIT, KITLG, KLK10, KPNA2, KRAS2, KRT18, KRT2A, KRT9, LAMB1, LAMP2, LCK, LCN2, LEP, LITAF, LRPAP1, LTF, LYN, LZTR1, MADH1, MAP2K2, MAP3K8, MAPK12, MAPK13, MAPKAPK3, MAPRE1, MARS, MAS1, MCC, MCM2, MCM4, MDM2, MDM4, MET, MGST1, MICB, MLLT3, MME, MMP1, MMP14, MMP17, MMP2, MNDA, MSH2, MSH6, MT3, MYB, MYBL1, MYBL2, MYC, MYCL1, MYCN, MYD88, MYL9, MYLK, NEO1, NF1, NF2, NFKB1, NFKB2, NFSF7, NID, NINE, NMBR, NME1, NME2, NME3, NOTCH1, NOTCH2, NOTCH4, NPM1, NQO1, NRID1, NR2F1, NR2F6, NRAS, NRG1, NSEP1, OSM, PA2G4, PABPC1, PCNA, PCTK1, PCTK2, PCTK3, PDGFA, PDGFB, PDGFRA, PDPK1, PEA 15, PFDN4, PFDN5, PGAM1, PHB, PIK3CA, PIK3CB, PIK3CG, PIM1, PKM2, PKMYT1, PLK2, PPARD, PPARG, PPIH, PPP1CA, PPP2R5A, PRDX2, PRDX4, PRKARIA, PRKCBP1, PRNP, PRSS15, PSMA 1, PTCH, PTEN, PTGS1, PTMA, PTN, PTPRN, RAB5A, RAC1, RAD50, RAF1, RALBP1, RAPlA, RARA, RARB, RASGRF1, RB1, RBBP4, RBL2, REA, REL, RELA, RELB, RET, RFC2, RGS19, RHOA, RHOB, RHOC, RHOD, RIPK1, RPN2, RPS6 KB1, RRM1, SARS, SELENBP1, SEMA3C, SEMA4D, SEPP1, SERPINH1, SFN, SFPQ, SFRS7, SHB, SHH, SIAH2, SIVA, SIVA TP53, SKI, SKIL, SLC16A1, SLC1A4, SLC20A1, SMO, sphingomyelin phosphodiesterase 1 (SMPD1), SNAI2, SND1, SNRPB2, SOCS1, SOCS3, SOD1, SORT1, SPINT2, SPRY2, SRC, SRPX, STAT1, STAT2, STAT3, STAT5B, STC1, TAF1, TBL3, TBRG4, TCF1, TCF7L2, TFAP2C, TFDP1, TFDP2, TGFA, TGFB1, TGFBI, TGFBR2, TGFBR3, THBS1, T1E, TIMP1, TIMP3, TJP1, TK1, TLE1, TNF, TNFRSFOA, TNFRSF10B, TNFRSF1A, TNFRSF1B, TNFRSF6, TNFSF7, TNK1, TOB1, TP53, TP53BP2, TP5313, TP73, TPBG, TPT1, TRADD, TRAM1, TRRAP, TSG101, TUFM, TXNRD1, TYRO3, UBC, UBE2L6, UCHL1, USP7, VDAC1, VEGF, VHL, VIL2, WEE1, WNT1, WNT2, WNT2B, WNT3, WNT5A, WT1, XRCC1, YES1, YWHAB, YWHAZ, ZAP70, and ZNF9.

A rAAV vector may comprise as a transgene, a nucleic acid encoding a protein or functional RNA that modulates apoptosis. The following is a non-limiting list of genes associated with apoptosis and nucleic acids encoding the products of these genes and their homologues and encoding small interfering nucleic acids (e.g., shRNAs, miRNAs) that inhibit the expression of these genes and their homologues are useful as transgenes in certain embodiments of the invention: RPS27A, ABL1, AKT1, APAF1, BAD, BAG1, BAG3, BAG4, BAK1, BAX, BCL10, BCL2, BCL2A1, BCL2L1, BCL2L10, BCL2L11, BCL2L12, BCL2L13, BCL2L2, BCLAF1, BFAR, BID, BIK, NAIP, BIRC2, BIRC3, XIAP, BIRC5, BIRC6, BIRC7, BIRC8, BNIP1, BNIP2, BNIP3, BNIP3L, BOK, BRAF, CARD10, CARD1, NLRC4, CARD14, NOD2, NOD1, CARD6, CARDS, CARDS, CASP1, CASP10, CASP14, CASP2, CASP3, CASP4, CASP5, CASP6, CASP7, CASP8, CASP9, CFLAR, CIDEA, CIDEB, CRADD, DAPK1, DAPK2, DFFA, DFFB, FADD, GADD45A, GDNF, HRK, IGF1R, LTA, LTBR, MCL1, NOL3, PYCARD, RIPK1, RIPK2, TNF, TNFRSFOA, TNFRSF10B, TNFRSF10C, TNFRSF10D, TNFRSF11B, TNFRSF12A, TNFRSF14, TNFRSF19, TNFRSFIA, TNFRSF1B, TNFRSF21, TNFRSF25, CD40, FAS, TNFRSF6B, CD27, TNFRSF9, TNFSF10, TNFSF14, TNFSF18, CD40LG, FASLG, CD70, TNFSF8, TNFSF9, TP53, TP53BP2, TP73, TP63, TRADD, TRAF1, TRAF2, TRAF3, TRAF4, and TRAF5.

Useful transgene products also include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

The following non-limiting list of miRNA genes, and their homologues, are useful as transgenes or as targets for small interfering nucleic acids encoded by transgenes (e.g., miRNA sponges, antisense oligonucleotides, TuD RNAs) in certain embodiments of the methods: hsa-let-7a, hsa-let-7a*, hsa-let-7b, hsa-let-7b*, hsa-let-7c, hsa-let-7c*, hsa-let-7d, hsa-let-7d*, hsa-let-7e, hsa-let-7e*, hsa-let-7f, hsa-let-7f-1*, hsa-let-7f-2*, hsa-let-7g, hsa-let-7g*, hsa-let-71, hsa-let-71*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-181a*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-2%-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-296-1*, hsa-miR-296-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302e, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30c, hsa-miR-30e*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-43*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518e, hsa-miR-518e*, hsa-miR-518f, hsa-miR-518f* hsa-miR-519a, hsa-miR-519b-3p, hsa-miR-519c-3p, hsa-miR-519d, hsa-miR-519e, hsa-miR-519e*, hsa-miR-520a-3p, hsa-miR-520a-5p, hsa-miR-520b, hsa-miR-520c-3p, hsa-miR-520d-3p, hsa-miR-520d-5p, hsa-miR-520c, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-5486-5p, hsa-miR-548c-3p, hsa-miR-548c-5p, hsa-miR-548d-3p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-5481, hsa-miR-548m, hsa-miR-548n, hsa-miR-548o, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609 hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96* hsa-miR-98, hsa-miR-99a, hsa-miR-99a*, hsa-miR-99b, and hsa-miR-99b*. For example, miRNA targeting chromosome 8 open reading frame 72 (C9orf72) which expresses superoxide dismutase (SOD1), associated with amyotrophic lateral sclerosis (ALS) may be of interest.

A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (derepress the polypeptide). In one embodiment, derepression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, a small interfering nucleic acid that is substantially complementary to a miRNA is one that is capable of hybridizing with a miRNA, and blocking the miRNA's activity. In some embodiments, a small interfering nucleic acid that is substantially complementary to a miRNA is an small interfering nucleic acid that is complementary with the miRNA at all but 1, 2, 3, 4, 5, 6, 7, 8, 9, 10, 11, 12, 13, 14, 15, 16, 17, or 18 bases. A “miRNA Inhibitor” is an agent that blocks miRNA function, expression and/or processing. For instance, these molecules include but are not limited to microRNA specific antisense, microRNA sponges, tough decoy RNAs (TuD RNAs) and microRNA oligonucleotides (double-stranded, hairpin, short oligonucleotides) that inhibit miRNA interaction with a Drosha complex.

Still other useful transgenes may include those encoding immunoglobulins which confer passive immunity to a pathogen. An “immunoglobulin molecule” is a protein containing the immunologically-active portions of an immunoglobulin heavy chain and immunoglobulin light chain covalently coupled together and capable of specifically combining with antigen. Immunoglobulin molecules are of any type (e.g., IgG, IgE, IgM, IgD, IgA and IgY), class (e.g., IgG1, IgG2, IgG3, IgG4, IgA1 and IgA2) or subclass. The terms “antibody” and “immunoglobulin” may be used interchangeably herein.

An “immunoglobulin heavy chain” is a polypeptide that contains at least a portion of the antigen binding domain of an immunoglobulin and at least a portion of a variable region of an immunoglobulin heavy chain or at least a portion of a constant region of an immunoglobulin heavy chain. Thus, the immunoglobulin derived heavy chain has significant regions of amino acid sequence homology with a member of the immunoglobulin gene superfamily. For example, the heavy chain in a Fab fragment is an immunoglobulin-derived heavy chain.

An “immunoglobulin light chain” is a polypeptide that contains at least a portion of the antigen binding domain of an immunoglobulin and at least a portion of the variable region or at least a portion of a constant region of an immunoglobulin light chain. Thus, the immunoglobulin-derived light chain has significant regions of amino acid homology with a member of the immunoglobulin gene superfamily.

An “immunoadhesin” is a chimeric, antibody-like molecule that combines the functional domain of a binding protein, usually a receptor, ligand, or cell-adhesion molecule, with immunoglobulin constant domains, usually including the hinge and Fc regions.

A “fragment antigen-binding” (Fab) fragment” is a region on an antibody that binds to antigens. It is composed of one constant and one variable domain of each of the heavy and the light chain.

The anti-pathogen construct is selected based on the causative agent (pathogen) for the disease against which protection is sought. These pathogens may be of viral, bacterial, or fungal origin, and may be used to prevent infection in humans against human disease, or in non-human mammals or other animals to prevent veterinary disease.

The rAAV may include genes encoding antibodies, and particularly neutralizing antibodies against a viral pathogen. Such anti-viral antibodies may include anti-influenza antibodies directed against one or more of Influenza A, Influenza B, and Influenza C. The type A viruses are the most virulent human pathogens. The serotypes of influenza A which have been associated with pandemics include, H1N1, which caused Spanish Flu in 1918, and Swine Flu in 2009; H2N2, which caused Asian Flu in 1957; H3N2, which caused Hong Kong Flu in 1968; H5N1, which caused Bird Flu in 2004; H7N7; HN2; H9N2; H7N2; H7N3; and H10N7. Other target pathogenic viruses include, arenaviruses (including funin, machupo, and Lassa), filoviruses (including Marburg and Ebola), hantaviruses, picornoviridae (including rhinoviruses, echovirus), coronaviruses, paramyxovirus, morbillivirus, respiratory synctial virus, togavirus, coxsackievirus, JC virus, parvovirus B19, parainfluenza, adenoviruses, reoviruses, variola (Variola major (Smallpox)) and Vaccinia (Cowpox) from the poxvirus family, and varicella-zoster (pseudorabies). Viral hemorrhagic fevers are caused by members of the arenavirus family (Lassa fever) (which family is also associated with Lymphocytic choriomeningitis (LCM)), filovirus (ebola virus), and hantavirus (puremala). The members of picomavirus (a subfamily of rhinoviruses), are associated with the common cold in humans. The coronavirus family, which includes a number of non-human viruses such as infectious bronchitis virus (poultry), porcine transmissible gastroenteric virus (pig), porcine hemagglutinatin encephalomyelitis virus (pig), feline infectious peritonitis virus (cat), feline enteric coronavirus (cat), canine coronavirus (dog). The human respiratory coronaviruses, have been putatively associated with the common cold, non-A, B or C hepatitis, and sudden acute respiratory syndrome (SARS). The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus (RSV). The parvovirus family includes feline parvovirus (feline enteritis), feline panleukopenia virus, canine parvovirus, and porcine parvovirus. The adenovirus family includes viruses (EX, AD7, ARD, O.B.) which cause respiratory disease. Thus, in certain embodiments, a rAAV vector as described herein may be engineered to express an anti-ebola antibody, e.g., 2G4, 4G7, 13C6, an anti-influenza antibody, e.g., FI6, CR8033, and anti-RSV antibody, e.g, palivizumab, motavizumab.

A neutralizing antibody construct against a bacterial pathogen may also be selected for use in the present invention. In one embodiment, the neutralizing antibody construct is directed against the bacteria itself. In another embodiment, the neutralizing antibody construct is directed against a toxin produced by the bacteria. Examples of airborne bacterial pathogens include, e.g., Neisseria meningitidis (meningitis), Klebsiella pneumonia (pneumonia), Pseudomonas aeruginosa (pneumonia), Pseudomonas pseudomallei (pneumonia), Pseudomonas mallei (pneumonia), Acinetobacter (pneumonia), Moraxella catarrhalis, Moraxella lacunata, Alkaligenes, Cardiobacterium, Haemophilus influenzae (flu), Haemophilus parainfluenzae, Bordetella pertussis (whooping cough), Francisella tularensis (pneumonia/fever), Legionella pneumonia (Legionnaires disease), Chlamydia psittaci (pneumonia), Chlamydia pneumoniae (pneumonia), Mycobacterium tuberculosis (tuberculosis (TB)), Mycobacterium kansasii (TB), Mycobacterium avium (pneumonia), Nocardia asteroides (pneumonia), Bacillus anthracis (anthrax), Staphylococcus aureus (pneumonia), Streptococcus pyogenes (scarlet fever), Streptococcus pneumoniae (pneumonia), Corynebacteria diphtheria (diphtheria), Mycoplasma pneumoniae (pneumonia).

The rAAV may include genes encoding antibodies, and particularly neutralizing antibodies against a bacterial pathogen such as the causative agent of anthrax, a toxin produced by Bacillius anthracis. Neutralizing antibodies against protective agent (PA), one of the three peptides which form the toxoid, have been described. The other two polypeptides consist of lethal factor (LF) and edema factor (EF). Anti-PA neutralizing antibodies have been described as being effective in passively immunization against anthrax. See, e.g., U.S. Pat. No. 7,442,373; R. Sawada-Hirai et al, J Immune Based Ther Vaccines. 2004; 2: 5. (on-line 2004 May 12). Still other anti-anthrax toxin neutralizing antibodies have been described and/or may be generated. Similarly, neutralizing antibodies against other bacteria and/or bacterial toxins may be used to generate an AAV-delivered anti-pathogen construct as described herein.

Antibodies against infectious diseases may be caused by parasites or by fungi, including, e.g., Aspergillus species, Absidia corymbifera, Rhixpus stolonifer, Mucor plumbeaus, Cryptococcus neoformans, Histoplasm capsulatum, Blastomyces dermatitidis, Coccidioides immitis, Penicillium species, Micropolysporafaeni, Thermoactinomyces vulgaris, Alternaria alternate, Cladosporium species, Helminthosporium, and Stachybotrys species.

The rAAV may include genes encoding antibodies, and particularly neutralizing antibodies, against pathogenic factors of diseases such as Alzheimer's disease (AD), Parkinson's disease (PD), GBA-associated-Parkinson's disease (GBA-PD), Rheumatoid arthritis (RA), Irritable bowel syndrome (IBS), chronic obstructive pulmonary disease (COPD), cancers, tumors, systemic sclerosis, asthma and other diseases. Such antibodies may be, without limitation, e.g., alpha-synuclein, anti-vascular endothelial growth factor (VEGF) (anti-VEGF), anti-VEGFA, anti-PD-1, anti-PDL1, anti-CTLA-4, anti-TNF-alpha, anti-IL-17, anti-IL-23, anti-IL-21, anti-IL-6, anti-IL-6 receptor, anti-IL-5, anti-IL-7, anti-Factor XII, anti-IL-2, anti-HIV, anti-IgE, anti-tumour necrosis factor receptor-1 (TNFR1), anti-notch 2/3, anti-notch 1, anti-OX40, anti-erb-b2 receptor tyrosine kinase 3 (ErbB3), anti-ErbB2, anti-beta cell maturation antigen, anti-B lymphocyte stimulator, anti-CD20, anti-HER2, anti-granulocyte macrophage colony-stimulating factor, anti-oncostatin M (OSM), anti-lymphocyte activation gene 3 (LAG3) protein, anti-CCL20, anti-serum amyloid P component (SAP), anti-prolyl hydroxylase inhibitor, anti-CD38, anti-glycoprotein IIb/IIIa, anti-CD52, anti-CD30, anti-IL-1beta, anti-epidermal growth factor receptor, anti-CD25, anti-RANK ligand, anti-complement system protein C5, anti-CDIIa, anti-CD3 receptor, anti-alpha-4 (α4) integrin, anti-RSV F protein, and anti-integrin a₄β₇. Still other pathogens and diseases will be apparent to one of skill in the art. Other suitable antibodies may include those useful for treating Alzheimer's Disease, such as, e.g., anti-beta-amyloid (e.g., crenezumab, solanezumab, aducanumab), anti-beta-amyloid fibril, anti-beta-amyloid plaques, anti-tau, a bapineuzamab, among others. Other suitable antibodies for treating a variety of indications include those described, e.g., in PCT/US2016/058968, filed 27 Oct. 2016, published as WO 2017/075119A1.

rAAV Vector Production

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

In one embodiment, the expression cassettes described herein are engineered into a genetic element (e.g., a shuttle plasmid) which transfers the immunoglobulin construct sequences carried thereon into a packaging host cell for production a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, N.Y. (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See. e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See. e.g., Gao, et al, Proc. Natl. Acad. Sci. U.S.A. 100 (10), 6081-6086 (2003) and US 2013/0045186A1.

In one embodiment, a production cell culture useful for producing a recombinant AAV is provided. Such a cell culture contains a nucleic acid which expresses the AAV capsid protein in the host cell; a nucleic acid molecule suitable for packaging into the AAV capsid, e.g., a vector genome which contains AAV ITRs and a non-AAV nucleic acid sequence encoding a gene product operably linked to sequences which direct expression of the product in a host cell; and sufficient AAV rep functions and adenovirus helper functions to permit packaging of the nucleic acid molecule into the recombinant AAV capsid. In one embodiment, the cell culture is composed of mammalian cells (e.g., human embryonic kidney 293 cells, among others) or insect cells (e.g., baculovirus).

Optionally the rep functions are provided by an AAV other than the AAV providing the capsid. For example the rep may be, but is not limited to, AAV1 rep protein, AAV2 rep protein, AAV3 rep protein, AAV4 rep protein, AAV5 rep protein, AAV6 rep protein, AAV7 rep protein, AAV8 rep protein; or rep 78, rep 68, rep 52, rep 40, rep68/78 and rep40/52; or a fragment thereof; or another source. Optionally, the rep and cap sequences are on the same genetic element in the cell culture. There may be a spacer between the rep sequence and cap gene. Any of these AAV or mutant AAV capsid sequences may be under the control of exogenous regulatory control sequences which direct expression thereof in a host cell.

In one embodiment, cells are manufactured in a suitable cell culture (e.g., HEK 293) cells. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Application No. PCT/US2016/065970, filed Dec. 9, 2016 and its priority documents, US Patent Application Nos. 62/322,071, filed Apr. 13, 2016 and 62/226,357, filed Dec. 11, 2015 and entitled “Scalable Purification Method for AAV9”, which is incorporated by reference herein. Purification methods for AAV8, International Patent Application No. PCT/US2016/065976, filed Dec. 9, 2016 and its priority documents US Patent Application Nos. 62/322,098, filed Apr. 13, 2016 and 62/266,341, filed Dec. 11, 2015, and rh10, International Patent Application No. PCT/US16/66013, filed Dec. 9, 2016 and its priority documents, U.S. Patent Application No. 62/322,055, filed Apr. 13, 2016 and 62/266,347, entitled “Scalable Purification Method for AAVrh10”, also filed Dec. 11, 2015, and for AAV1, International Patent Application No. PCT/US2016/065974, filed Dec. 9, 2016 and its priority documents US Patent Application Nos. 62/322,083, filed Apr. 13, 2016 and 62/26,351, for “Scalable Purification Method for AAV1”, filed Dec. 11, 2015, are all incorporated by reference herein.

To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

In brief, the method for separating rAAV particles having packaged genomic sequences from genome-deficient AAV intermediates involves subjecting a suspension comprising recombinant AAV viral particles and AAV capsid intermediates to fast performance liquid chromatography, wherein the AAV viral particles and AAV intermediates are bound to a strong anion exchange resin equilibrated at a high pH, and subjected to a salt gradient while monitoring eluate for ultraviolet absorbance at about 260 and about 280. The pH may be adjusted depending upon the AAV selected. See, e.g., WO2017/160360 (AAV9), WO2017/100704 (AAVrh10), WO 2017/100676 (e.g., AAV8), and WO 2017/100674 (AAV1)] which are incorporated by reference herein. In this method, the AAV full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2 serotype. Under these ionic conditions, a significant percentage of residual cellular DNA and proteins flow through the column, while AAV particles are efficiently captured.

### Compositions and Uses

Provided herein are compositions containing at least one rAAV stock (e.g., an rAAV stock or a mutant rAAV stock) and an optional carrier, excipient and/or preservative. An rAAV stock refers to a plurality of rAAV vectors which are the same, e.g., such as in the amounts described below in the discussion of concentrations and dosage units.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery), lung, heart, eye, kidney,), oral, inhalation, intranasal, intrathecal, intratracheal, intraarterial, intraocular, intravenous, intramuscular, subcutaneous, intradermal, and other parental routes of administration. Routes of administration may be combined, if desired.

Dosages of the viral vector will depend primarily on factors such as the condition being treated, the age, weight and health of the patient, and may thus vary among patients. For example, a therapeutically effective human dosage of the viral vector is generally in the range of from about 25 to about 1000 microliters to about 100 mL of solution containing concentrations of from about 1×10⁹ to 1×10¹⁶ genomes virus vector. The dosage will be adjusted to balance the therapeutic benefit against any side effects and such dosages may vary depending upon the therapeutic application for which the recombinant vector is employed. The levels of expression of the transgene can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10, 5×10⁹, 6×10⁹, 7×10⁹, 8×10⁹, or 9×10⁹GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In certain embodiments, the dose may be in the range of about 1×10⁹ GC/g brain mass to about 1×10¹² GC/g brain mass. In certain embodiments, the dose may be in the range of about 3×10¹⁰ GC/g brain mass to about 3×10¹¹ GC/g brain mass. In certain embodiments, the dose may be in the range of about 5×10¹⁰ GC/g brain mass to about 1.85×10¹¹ GC/g brain mass.

In one embodiment, the viral constructs may be delivered in doses of from at least about least 1×10⁹ GCs to about 1×10¹⁵, or about 1×10¹¹ to 5×10¹³ GC. Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

The above-described recombinant vectors may be delivered to host cells according to published methods. The rAAV, preferably suspended in a physiologically compatible carrier, may be administered to a human or non-human mammalian patient. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of about 6.8 to about 7.2 may be desired. However, other pHs within the broadest ranges and these subranges may be selected for other route of delivery.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo. A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy-oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension. In one example, the formulation may contain, e.g., buffered saline solution comprising one or more of sodium chloride, sodium bicarbonate, dextrose, magnesium sulfate (e.g., magnesium sulfate.7H₂O), potassium chloride, calcium chloride (e.g., calcium chloride.2H₂O), dibasic sodium phosphate, and mixtures thereof, in water. Suitably, for intrathecal delivery, the osmolarity is within a range compatible with cerebrospinal fluid (e.g., about 275 to about 290); see, e.g., emedicine.medscape.com/article/2093316-overview. Optionally, for intrathecal delivery, a commercially available diluent may be used as a suspending agent, or in combination with another suspending agent and other optional excipients. See, e.g., Elliotts B® solution [Lukare Medical]. In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intrathecal delivery.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna.

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube.

In one aspect, the vectors provided herein may be administered intrathecally via the method and/or the device. See, e.g., WO 2017/181113, which is incorporated by reference herein. Alternatively, other devices and methods may be selected. The method comprises the steps of advancing a spinal needle into the cisterna magna of a patient, connecting a length of flexible tubing to a proximal hub of the spinal needle and an output port of a valve to a proximal end of the flexible tubing, and after said advancing and connecting steps and after permitting the tubing to be self-primed with the patient's cerebrospinal fluid, connecting a first vessel containing an amount of isotonic solution to a flush inlet port of the valve and thereafter connecting a second vessel containing an amount of a pharmaceutical composition to a vector inlet port of the valve. After connecting the first and second vessels to the valve, a path for fluid flow is opened between the vector inlet port and the outlet port of the valve and the pharmaceutical composition is injected into the patient through the spinal needle, and after injecting the pharmaceutical composition, a path for fluid flow is opened through the flush inlet port and the outlet port of the valve and the isotonic solution is injected into the spinal needle to flush the pharmaceutical composition into the patient.

This method and this device may each optionally be used for intrathecal delivery of the compositions provided herein. Alternatively, other methods and devices may be used for such intrathecal delivery.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% (10%) from the reference given, unless otherwise specified.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

The term “expression” is used herein in its broadest meaning and comprises the production of RNA or of RNA and protein. With respect to RNA, the term “expression” or “translation” relates in particular to the production of peptides or proteins. Expression may be transient or may be stable.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199(3): p. 381-390, which is incorporated by reference herein.

As used herein, an “expression cassette” refers to a nucleic acid molecule which comprises a coding sequence, promoter, and may include other regulatory sequences therefor, which cassette may be delivered via a genetic element (e.g., a plasmid) to a packaging host cell and packaged into the capsid of a viral vector (e.g., a viral particle). Typically, such an expression cassette for generating a viral vector contains the coding sequence for the gene product described herein flanked by packaging signals of the viral genome and other expression control sequences such as those described herein.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See. e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

As used herein, the term “operably linked” refers to both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

In many instances, rAAV particles are referred to as DNase resistant. However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a transgene to a host cell and protects these packaged genomic sequences from degradation (digestion) during nuclease incubation steps designed to remove contaminating nucleic acids which may be present from the production process.

The term “translation” in the context of the present invention relates to a process at the ribosome, wherein an mRNA strand controls the assembly of an amino acid sequence to generate a protein or a peptide.

As used throughout this specification and the claims, the terms “comprising” and “including” are inclusive of other components, elements, integers, steps and the like. Conversely, the term “consisting” and its variants are exclusive of other components, elements, integers, steps and the like.

As described above, the term “about” when used to modify a numerical value means a variation of 10%, unless otherwise specified.

The following examples are illustrative only and are not intended to limit the present invention.

### EXAMPLES

The following examples report the extensive deamidation of AAV8 and 7 additional diverse AAV serotypes, with supporting evidence from structural, biochemical, and mass spectrometry approaches. The extent of deamidation at each site was dependent on the age of the vector and multiple primary-sequence and 3D-structural factors, but was largely independent of the conditions of vector recovery and purification. We demonstrate the potential for deamidation to impact vector transduction activity, and correlate an early timepoint loss in vector activity to rapidly progressing, spontaneous deamidation at several AAV8 asparagines. We explore mutational strategies that stabilize side-chain amides, improving vector transduction and reducing the lot-to-lot molecular variability that is a key concern in biologics manufacturing. This study illustrates a previously unknown aspect of AAV capsid heterogeneity and highlights its importance in the development of these vectors for gene therapy.

Example 1 below provide the characterization of post-translational modifications to the AAV8 vector capsid by one- and two-dimensional gel electrophoresis, mass spectrometry, and de novo structural modeling. Following the identification of a number of putative deamidation sites on the capsid surface, we evaluate their impact on capsid structure and function both in vitro and in vivo. Example 1 further extends this analysis to AAV9 to determine if this phenomenon applies to serotypes other than AAV8, confirming that deamidation of the AAV capsid is not serotype specific. Examples 2 and 3 illustrates deamidation in further AAVs.

Example 4 relates to a novel epitope mapped on the AAV9 capsid.

### Example 1: Deamidation of Amino Acids on the Surface of Adeno-Associated Virus Capsids

A. Materials and Methods

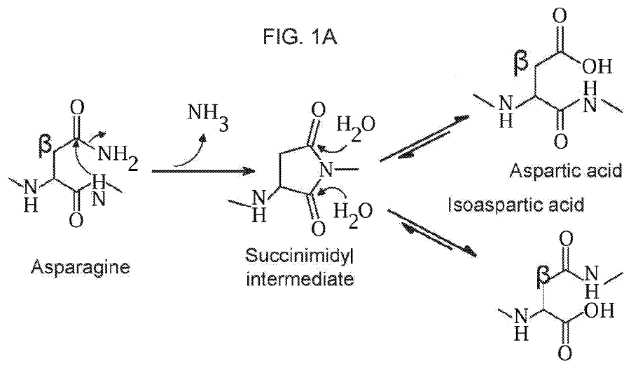

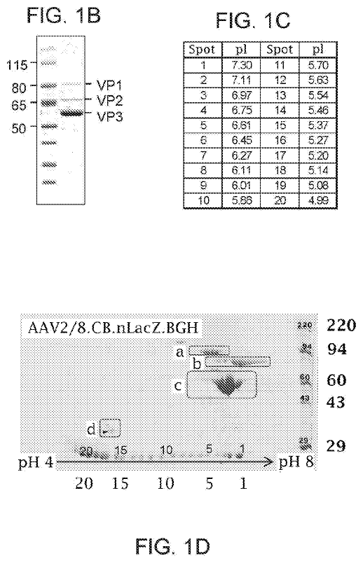

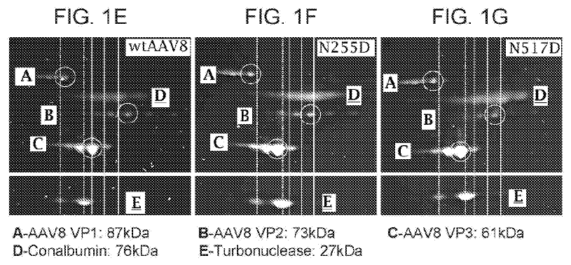

1. 1D and 2D Gel Electrophoresis For 1D SDS polyacrylamide gel electrophoresis (SDS-PAGE) analysis, we first denatured AAV vectors at 80° C. for 20 minutes in the presence of lithium dodecyl sulfate and reducing agent. Then, we ran them on a 4-12% Bis-Tris gel for 90 minutes at 200V and stained with Coomassie blue. For the data in FIG. 1A-FIG. 1D, Kendrick Laboratories, Inc. (Madison, Wis.) performed the 2D gel electrophoresis. For subsequent experiments, we performed 2D SDS-PAGE in-house. For this, we combined 3×10¹¹ GCs of AAV vector with 500U turbonuclease marker (Accelagen, San Diego, Calif.) in 150μL phosphate buffered saline (PBS) with 35 mM NaCl and 1 mM MgCl₂ and incubated at 37° C. for ten minutes. We next added nine volumes of absolute ethanol, vortexed the samples, and incubated them at −80° C. for at least two hours followed by incubation on ice for five minutes and centrifugation at maximum speed for 30 minutes at 15° C. We decanted the supernatant and air-dried the pellet, which we then resuspended in resuspension buffer #1 [0.15% SDS, 50 mM dithiothreitol (DTT), 10 mM Tris pH 7.5, and 1 μL pH6-9 ampholytes, ThermoFisher ZM0023, added day-of, in ddH₂O] and incubated undisturbed at room temperature. After 30 minutes, we flicked the sample tubes to mix them, added 1 μg chicken conalbumin marker (Sigma Aldrich, St. Louis, Mo.), and incubated the samples at 37° C. for 30 minutes, flicking to mix at 15 minutes. Samples were then transferred to 50° C. for 15-20 minutes, vortexed, incubated at 95° C. for 2.5 minutes, and allowed to cool before being centrifuged at maximum speed for one minute and briefly vortexed. We then mixed 10 μL of each sample with 140 μL resuspension buffer #2 (9.7M urea, 2% CHAPS, 0.002% bromophenol blue, and 0.05% ampholytes, described above, added day-of, in ddH₂O) and incubated at room temperature for ten minutes. We then applied the mixtures to pH 6-10 immobilized pH gradient (IPG) strips (ThermoFisher Waltham, Mass.) and ran them on the ZOOM IPGRunner system according to manufacturer's instructions. We used the following isoelectric focusing parameters: 100-1,000V for 120 minutes, 1,000-2,000V for 120 minutes, 2,000V for 120 minutes, limits of 0.1W and 0.05 mA per strip run. IPG strips were then reduced and loaded in a single-well 4-12% Bis-Tris gel and run in 1D as described above. We determined the relative migration of AAV VPs by comparison to internal control proteins turbonuclease (Accelagen, 27 kDa) and chicken egg white conalbumin (Sigma Aldrich, 76 kDa, pI 6.0-6.6).

2. Vector Production

The University of Pennsylvania Vector Core produced recombinant AAV vectors for 1D and 2D gel electrophoresis and mass spectrometry experiments and purified them by cesium chloride or iodixanol gradients as previously described. (Lock M, et al. Hum Gene Ther 2010; 21(10):1259-71; Gao G P, et al. Proc Natl Acad Sci USA. 2002; 99(18):11854-9). We produced the affinity purified vectors as follows: We grew HEK293 cells in ten 36-layer hyperstack vessels (Corning), co-transfected them with a mixture of vector genome plasmid (pAAV-LSP-IVS2.hFIXco-WPRE-bGH), trans plasmid containing AAV2 rep and AAV8 cap genes, and adenovirus helper plasmid. We used PEIpro (PolyPlus) as the transfection reagent. Five days post transfection, the supernatant was harvested, clarified through Sartoguard PES Midicap filters (Sartorious Stedim), and treated with benzonase (Millipore), after which we added salt to bring it to 0.6M. The clarified bulk harvest material was concentrated ten-fold by tangential flow filtration (TFF) and then diafiltered against four volumes of affinity column loading buffer. We captured vectors on a POROS CaptureSelect (ThermoFisher) affinity column and eluted the vector peak at low pH directly into neutralization buffer. We diluted the neutralized eluate into a high-pH binding buffer and loaded it onto an anion exchange polishing column (Cimultus QA-8; Bia Separations), where the preparation was enriched for genome-containing (full) particles. The full vector particles were eluted with a shallow salt elution gradient and neutralized immediately. Finally, we subjected the vector to a second round of TFF for final concentration and buffer exchange into formulation buffer (PBS+0.001% pluronic F-68).

We produced mutant vectors for in vitro assays by small-scale triple transfection of HEK293 cells in six-well plates. We mixed 5.6 μL of a 1 mg/mL polyethylenimine solution in 90 μL serum-free media with plasmid DNA (0.091 μg cis plasmid, 0.91 μg trans plasmid, 1.82 μg deltaF6 Ad-helper plasmid, in 90 μL serum-free media), incubated it at room temperature for 15 minutes, and added it to cells in and additional 0.8 mL of fresh serum-free media. The next day, we replaced 0.5 mL of the top media with full serum media. We harvested vector three days post-transfection by three freeze/thaw cycles followed by centrifugation to remove cell debris and supernatant harvest. Cis plasmid contained a transgene cassette encoding the firefly luciferase transgene under the control of the chicken-beta actin (CB7) promoter with the Promega chimeric intron and rabbit beta-globin (RBG) polyadenylation signal. Trans plasmid encoded the wtAAV8 cap gene; to generate mutant AAV8 cap variants, we used the Quikchange Lightning Mutagenesis kit (Agilent Technologies, Wilmington, Del.). Vector was titered as previously described. (Lock M, et al. Hum Gene Ther 2010; 21(10):1259-71).

For timecourse vector production experiments, we generated vector by medium-scale triple transfection of HEK293 cells in 15 cm tissue culture dishes. Per plate, we mixed 36 μL of a 1 mg/mL polyethylenimine solution in 2 mL serum-free media with plasmid DNA (0.6 μg cis plasmid, 5.8 μg trans plasmid, 11.6 μg deltaF6 Ad-helper plasmid), incubated it at room temperature for 15 minutes, and added it to cells at approximately 60% confluency on plates refreshed with 14 ml of serum-free media. The following day, we replaced 8 ml of the top media with fresh, full serum media. We harvested vector by collecting all top media, scraping cells from the dish and freezing this at −80° C. We recovered crude vector from the supernatant/cell mixture by applying 3 freeze/thaw cycles, and clarifying the lysate by centrifugation. We purified and concentrated the vector for mass spectrometry analysis by adding benzonase, 1M Tris pH7.5, and 5M NaCl to the clarified lysate to final concentrations of 20 mM Tris and 360 mM NaCl. We captured vectors on a 1 ml POROS CaptureSelect affinity column and eluted the vector peak at low pH directly into neutralization buffer. Fractions were analysed by absorbance at 280 nm, and the most concentrated fraction was subjected to mass spectrometry analysis.

For in vivo experiments, we produced vectors as previously described with a wtAAV8 capsid or with one of the 6 deamidation mutants; the transgene cassette included a CB7 promoter, PI intron, firefly luciferase transgene, and RBG polyadenylation signal (Lock M, et al. Hum Gene Ther 2010; 21(10):1259-71).

3. Mass Spec Run/Digest/Analysis

Materials: We purchased ammonium bicarbonate, DTT, iodoacetamide (IAM), and 180-enriched water (97.1% purity) from Sigma (St. Louis, Mo.); and acetonitrile, formic acid, trifluoroacetic acid (TFA), 8M guanidine hydrochloride (GndHCl), and trypsin from Thermo Fischer Scientific (Rockford, Ill.).

Trypsin digestion: We prepared stock solutions of 1M DTT and 1.0M iodoacetamide. Capsid proteins were denatured and reduced at 90° C. for ten minutes in the presence of 10 mM DTT and 2M GndHCl. We allowed the samples to cool to room temperature and then alkylated them with 30 mM IAM at room temperature for 30 minutes in the dark. We quenched the alkylation reaction with the addition of 1 mL DTT. We added 20 mM ammonium bicarbonate (pH 7.5-8) to the denatured protein solution at a volume that diluted the final GndHCl concentration to 200 mM. We added trypsin solution for a 1:20 trypsin to protein ratio and incubated at 37° C. overnight. After digestion, we added TFA to a final concentration of 0.5% to quench the digestion reaction.

For 180-water experiments, the capsid sample was first buffer exchanged into 100 mM ammonium bicarbonate prepared in 180-water using Zeba spin desalting columns (Thermo Scientific, Rockford, Ill.). To ensure a complete removal of the water in the sample, we performed the buffer exchange twice. We prepared stock solutions of 1M DTT and 1M IAM in 180-water. We followed the same denaturation, alkylation, and digestion steps as above with 180-water reagents and buffers.

Liquid chromatography tandem-mass spectrometry: We performed online chromatography with an Acclaim PepMap column (15 cm long, 300 μm inner diameter) and a Thermo UltiMate 3000 RSLC system (Thermo Fisher Scientific) coupled to a Q Exactive HF with a NanoFlex source (Thermo Fisher Scientific). During online analysis, the column temperature was maintained at a temperature of 35° C. We separated peptides with a gradient of mobile phase A (MilliQ water with 0.1% formic acid) and mobile phase B (acetonitrile with 0.1% formic acid). We ran the gradient from 4% B to 6% B over 15 minutes, to 10% B for 25 minutes (40 minutes total), and then to 30% B for 46 minutes (86 minutes total). We loaded the samples directly to the column. The column size was 75 cm×15 um I.D. and was packed with 2 micron C18 media (Acclaim PepMap). Due to the loading, lead-in, and washing steps, the total time for each liquid chromatography tandem-mass spectrometry run was about two hours.

We acquired mass spectrometry data using a data-dependent top-20 method on the Q Exactive HF mass spectrometer, dynamically choosing the most abundant not-yet-sequenced precursor ions from the survey scans (200-2000 m/z). We performed sequencing via higher energy collisional dissociation fragmentation with a target value of 1e5 ions determined with predictive automatic gain control; we performed isolation of precursors with a window of 4m/z. We acquired survey scans at a resolution of 120,000 at 200m/z. We set the resolution for HCD spectra to 30,000 at m/z200 with a maximum ion injection time of 50 ms and a normalized collision energy of 30. We set the S-lens RF level to 50, which gave optimal transmission of the m/z region occupied by the peptides from our digest. We excluded precursor ions with single, unassigned, or six and higher charge states from fragmentation selection.

Data processing: We used BioPharma Finder 1.0 software (Thermo Fischer Scientific) to analyze all data acquired. For peptide mapping, we performed searches using a single-entry protein FASTA database with carbamidomethylation set as a fixed modification, and oxidation, deamidation, and phosphorylation set as variable modifications. We used a 10 ppm mass accuracy, a high protease specificity, and a confidence level of 0.8 for tandem-mass spectrometry spectra. Mass spectrometric identification of deamidated peptides is relatively straightforward, as deamidation adds to the mass of intact molecule +0.984 Da (the mass difference between —OH and —NH2 groups). We determined the percent deamidation of a particular peptide by dividing the mass area of the deamidated peptide by the sum of the area of the deamidated and native peptides. Considering the number of possible deamidation sites, isobaric species that are deamidated at different sites may co-migrate at a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent of the site of deamidation. This approach allows the definition of the specific sites involved in deamidation and the potential combinations involved in deamidation.

Secondary data processing: Secondary analysis of raw mass spectrometry was performed at the University of Maryland, Baltimore County using the following method. Peaks Studio v5.3 software (Bioinformatics Solutions Inc.) was used for all mass spectrometry analysis. Data refinement of the raw data files was performed with the following parameters: a precursor m/z tolerance of ≤10 ppm, and precursor charge state with a minimum of 2, maximum of 4. De novo sequencing of the input spectrum was performed using the Peaks algorithm with a precursor ion error tolerance of 10 ppm and product ion error tolerances of 0.1 Da. The digestion enzyme was set as trypsin, the variable modifications were oxidation, phosphorylation, and deamidation, and the fixed modification was carbamidomethylation of cysteine.

4. Structural Analysis of the AAV Capsid

We obtained the AAV8 atomic coordinates, structural factors, and associated capsid model from the RCSB Protein Data Bank (PDB ID: 3RA8). We performed structure refinement and generated an electron density independent of the primary amino acid sequence of AAV8 VP3 for use in three-dimensional (3D) structural analysis of the capsid. We performed this analysis in order to observe the isoaspartic acid electron density in the AAV8 capsid that was not biased by the expected primary sequence of AAV8 VP3. Using the resulting structure, we modeled the four asparagines in the AAV8 VP3 primary sequence with N+1 glycines as isoaspartic acids and then refined the AAV8 capsid structure using Crystallography and NMR System (CNS) software by strictly imposing the icosahedral non-crystallographic matrices using the standard refinement protocol (Brunger A T, et al. Acta Crystallogr D Biol Crystallogr 1998; 54(Pt 5):905-21). We obtained a structural model of isoaspartic acid from the HIC-UP database, followed by generation of a molecular dictionary in PRODRG for structure refinement (Kleywegt G J Acta Crystallogr D Biol Crystallogr 2007; 63(Pt 1):94-100). We then calculated the average electron density map of the AAV8 capsid (also in CNS) and visualized it using COOT software, followed by minor adjustments of the resulting model to fit the modeled isoaspartic acid residues into the electron density map (Emsley P and Cowtan K Acta Crystallogr D Biol Crystallogr 2004; 60(Pt 12 Pt 1):2126-32). We repeated this protocol to additionally model N512 in the AAV9 VP3 primary sequence with N+1 glycines (PDB ID: 3UX1). We generated all figures using COOT, PyMol, and UCSF Chimera (Emsley P and Cowtan K Acta Crystallogr D Biol Crystallogr 2004; 60(Pt 12 Pt 1):2126-32; DeLano W L PyMOL: An Open-Source Molecular Graphics Tool Vol. 40, 2002:82-92; Pettersen E F, et al. J Comput Chem 2004; 25(13):1605-12). We obtained a number of structures of previously identified deamidated proteins (PDB IDs: 1DY5, 4E7G, 1RTU, 1W9V, 4E7D, and 1C9D) for comparison of their electron density map for deamidated isoaspartic acid residues with our modeled isoaspartic acid residues from AAV8 and AAV9 (Rao F V, et al. Chem Biol 2005; 12(1):65-76; Noguchi S, et al. Biochemistry 1995; 34(47):15583-91; Esposito L, et al. J Mol Biol 2000; 297(3):713-32).

We determined temperature factors for deamidated residues by averaging the temperature factors for each atom of each asparagine residue reported in the AAV8 or AAV9 crystal structure atomic coordinates (PDB ID: 3RA8, 3UX1).

5. Animal Studies

The Institutional Animal Care and Use Committee of the University of Pennsylvania approved all animal procedures. To evaluate vector performance, we injected eight-week-old C57BL/6 mice intravenously via tail vein injection with 3e10 GCs of wtAAV8 or capsid mutant vector in a volume of 100 μL. All mice were sacrificed at day 14. For in vivo evaluation of luciferase expression, mice (˜20g) were anesthetized and injected intraperitoneally with 200 μL or 15 mg/mL luciferin substrate (Perkin Elmer, Waltham, Mass.). Mice were imaged five minutes after luciferin administration and imaged via an IVIS Xenogen In Vivo Imaging System. We used Living Image 3.0 software to quantify signal in the described regions of interest. We took measurements at days 7 and 14.

6. Evaluation of Mutant Vector Titer and In Vitro Transduction Efficiency

We determined vector titers by qPCR of the DNAseI-resistant genomes. The qPCR primers anneal to the polyadenylation sequence of the packaged transgene. For in vitro evaluation of vector transduction efficiency by luciferase expression, we seeded 0.9e5 Huh7 cells/well in a black-walled 96-well plate in complete DMEM (10%/fetal bovine serum, 1% penicillin/streptomycin). The next day, we removed the media and replaced it with 50 μL crude or purified vector diluted in complete media. We tested 4 dilutions in a 3 fold dilution series for each crude vector sample. After 48 hours, we prepared luciferin (Promega, Madison, Wis.) in complete media at 0.3 μg/μL and added it to transduced cells in a volume of 50 μL. Results were read on a Biotek Clarity luminometer. We find that luciferase activity % GC added to target cells is constant over a wide range of GCs, but can become saturated at high MOIs. Thus we inspect the dilution series data (luminescent units vs GC) for linearity, exclude the highest point if saturation is evident, and calculate an average Luciferase/GC for values in the linear range of each assay for each variant. This yields a transduction efficiency value. The data are normalized to simplify comparison by setting the wt control to a value of 1. 7. Biodistribution

We extracted DNA from liver samples using the QIAamp DNA Mini Kit (Qiagen, Hilden, Germany), and then analyzed the DNA for vector GC by real-time PCR as described previously with a primer/probe set designed against the RBG polyadenylation signal of the transgene cassette (Chen S J, et al. Hum Gene Ther Clin Dev 2013; 24(4):154-60).

Primer Sequences for AAV8 Mutants

| Sequence | Description | |
|---|---|---|
| SED ID NO: 38 | CGACAACCGGGCAAAACcagAATAGC | QC mutagenic primers to |
| | AACTTTGCCTGG | change AAV8 N499 to Q |
| SED ID NO: 39 | CCAGGCAAAGTTGCTATTCTGGTTTTG | QC mutagenic primers to |
| | CCCGGTTGTCG | change AAV8 N499 to Q |
| SED ID NO: 40 | GACAACCGGGCAAAACgacAATAGCA | QC mutagenic primers to |
| | ACTTTGCCTG | change AAV8 N499 to D |
| SED ID NO: 41 | CAGGCAAAGTTGCTATTGTCGTTTTGC | QC mutagenic primers to |
| | CCGGTTGTC | change AAV8 N499 to D |
| SED ID NO: 42 | GGAGGCACGGCAcagACGCAGACTCTG | qc mutagenic primers to |
| | GG | change AAV8 N459 to Q |
| SED ID NO: 43 | CCCAGAGTCTGCGTCTGTGCCGTGCCT | qc mutagenic primers to |
| | CC | change AAV8 N459 to Q |
| SED ID NO: 44 | CAGGAGGCACGGCAgatACGCAGACTC | qc mutagenic primers to |
| | TGG | change AAV8 N459 to D |
| SED ID NO: 45 | CCAGAGTCTGCGTATCTGCCGTGCCTC | qc mutagenic primers to |
| | CTG | change AAV8 N459 to D |
| SED ID NO: 46 | ctcctcccgatgtcgcgttggagatttgc | AAV8 NA263 F |
| SED ID NO: 47 | gcaaatctccaacgcgacatcgggaggag | AAV8 NA263 R |
| SED ID NO: 48 | cccacggcctgactagcgttgttgagtgtta | AAV8 NA385 F |
| SED ID NO: 49 | taacactcaacaacgctagtcaggccgtggg | AAV8 NA385 R |
| SED ID NO: 50 | ggattagccaatgaatttcttgcattcagatggtatttggtcc | AAV8 NA514 F |
| SED ID NO: 51 | ggaccaaataccatctgaatgcaagaaattcattggctaatcc | AAV8 NA514 R |
| SED ID NO: 52 | tttgccaaaaatcaggatcgcgttactgggaaaaaaacg | AAV8 NA540 F |
| SED ID NO: 53 | cgtttatcccagtaacgcgatcctgatttttggcaaa | AAV8 NA540 R |
| SED ID NO: 54 | ggacccttcaacgcactcgacaagggg | AAV8 NA57 F |
| SED ID NO: 55 | ccccttgtcgagtgcgttgaagggtcc | AAV8 NA57 R |
| SED ID NO: 56 | tggctcctcccgatgtgctgttggagatttgcttg | AAV8 NS263 F |
| SED ID NO: 57 | caagcaaatctccaacagcacatcgggaggagcca | AAV8 NS263 R |
| SED ID NO: 58 | cccacggcctgactactgttgttgagtgttagg | AAV8 NS385 F |
| SED ID NO: 59 | cctaacactcaacaacagtagtcaggccgtggg | AAV8 NS385 R |
| SED ID NO: 60 | ttagccaatgaatttctgctattcagatggtatttggtcccagca | AAV8 NS514 F |
| | g | |
| SED ID NO: 61 | ctgctgggaccaaataccatctgaatagcagaaattcattggc | AAV8 NS514 R |
| | taa | |
| SED ID NO: 62 | ttgtttgccaaaaatcaggatgctgttactgggaaaaaaacgct | AAV8 NS540 F |
| | C | |
| SED ID NO: 63 | gagcgtttttttcccagtaacagcatcctgatttttggcaaacaa | AAV8 NS540 R |
| SED ID NO: 64 | ctcccccttgtcgaggctgttgaagggtccgag | AAV8 NS57 F |
| SED ID NO: 65 | ctcggacccttcaacagcctcgacaagggggag | AAV8 NS57 R |
| SED ID NO: 66 | cagcgactcatcaacGACaactggggattccg | QC primer for AAV8 |
| | | N305D |
| SED ID NO: 67 | ggaggcacggcaGATacgcagactctgg | QC primer for AAV8 |
| | | N459D |
| SED ID NO: 68 | gacaaccgggcaaaacGACaatagcaactttgcctg | QC primer for AAV8 |
| | | N499D |
| SED ID NO: 69 | ccatctgaatggaagaGATtcattggctaatcctggcatc | QC primer for AAV8 |
| | | N517D |
| SED ID NO: 70 | cgaagcccaaagccGACcagcaaaagcagg | QC primer for AAV8 |
| | | N35D |
| SED ID NO: 71 | gtacctgcggtatGACcacgccgacgcc | QC primer for AAV8 |
| | | N94D |
| SED ID NO: 72 | gatgctgagaaccggcGACaacttccagtttacttac | QC primer for AAV8 |
| | | N410D |
| SED ID NO: 73 | cagactctgggcttcagcGATggtgggcctaatacaatg | QC primer for AAV8 |
| | | Q467D |
| SED ID NO: 74 | ccaatcaggcaaagGACtggctgccaggac | QC primer for AAV8 |
| | | N479D |
| SED ID NO: 75 | cacggacggcGACttccacccgtctc | QC primer for AAV8 |
| | | N630D |
| SED ID NO: 76 | gatcctgatcaagGACacgcctgtacctgcg | QC primer for AAV8 |
| | | N653D |
| SED ID NO: 77 | gtacctcggacccttcCAGggactcgacaaggg | QC primer for AAV8 |
| | | N57Q |
| SED ID NO: 78 | ctacaagcaaatctccCAGgggacatcgggaggagc | QC primer for AAV8 |
| | | N263Q |
| SED ID NO: 79 | gctacctaacactcaacCAGggtagtcaggccgtgg | QC primer for AAV8 |
| | | N385Q |
| SED ID NO: 80 | gctgggaccaaataccatctgCAGggaagaaattcattgg | QC primer for AAV8 |
| | c | N514Q |
| SED ID NO: 81 | ggagcgtttttttcccagtCAGgggatcctgatttttggc | QC primer for AAV8 |
| | | N540Q |
| SED ID NO: 82 | cggaatccccagttgtcgttgatgagtcgctg | QC primer for AAV8 |
| | | N305D |
| SED ID NO: 83 | ccagagtctgcgtatctgccgtgcctcc | QC primer for AAV8 |
| | | N459D |
| SED ID NO: 84 | caggcaaagttgctattgtcgttttgcccggttgtc | QC primer for AAV8 |
| | | N499D |
| SED ID NO: 85 | gatgccaggattagccaatgaatctcttccattcagatgg | QC primer for AAV8 |
| | | N517D |
| SED ID NO: 86 | cctgcttttgctggtcggctttgggcttcg | QC primer for AAV8 |
| | | N35D |
| SED ID NO: 87 | ggcgtcggcgtggtcataccgcaggtac | QC primer for AAV8 |
| | | N94D |
| SED ID NO: 88 | gtaagtaaactggaagttgtcgccggttctcagcatc | QC primer for AAV8 |
| | | N410D |
| SED ID NO: 89 | cattgtattaggcccaccatcgctgaagcccagagtctg | QC primer for AAV8 |
| | | Q467D |
| SED ID NO: 90 | gtcctggcagccagtcctttgcctgattgg | QC primer for AAV8 |
| | | N479D |
| SED ID NO: 91 | gagacgggtggaagtcgccgtccgtg | QC primer for AAV8 |
| | | N630D |
| SED ID NO: 92 | cgcaggtacaggcgtgtccttgatcaggatc | QC primer for AAV8 |
| | | N653D |
| SED ID NO: 93 | gcagcgactcatcaacGACaactggggattccggc | alternative longer primer |
| | | to make AAV8 N305D by |
| | | qc mutagenesis |
| SED ID NO: 94 | GCCGGAATCCCCAGTTGTCGTTGATG | alternative longer primer |
| | AGTCGCTGC | to make AAV8 N305D by |
| | | qc mutagenesis |
| SED ID NO: 95 | cagcgactcatcaacGACaactggggattccggc | alternative longer primer |
| | | to make AAV8 N305D by |
| | | qc mutagenesis |
| SED ID NO: 96 | GCCGGAATCCCCAGTTGTCGTTGATG | alternative longer primer |
| | AGTCGCTG | to make AAV8 N305D by |
| | | qc mutagenesis |
| SED ID NO: 97 | gcgactcatcaacGACaactggggattccg | alternative shorter primer |
| | | to make AAV8 N305D by |
| | | qc mutagenesis |
| SED ID NO: 98 | CGGAATCCCCAGTTGTCGTTGATGAG | alternative shorter primer |
| | TCGC | to make AAV8 N305D by |
| | | qc mutagenesis |
| SED ID NO: 99 | ctctgggcttcagcGAAggtgggcctaatac | mutagenic QC primer to |
| | | make aav8 Q467E |
| SED ID NO: | GTATTAGGCCCACCTTCGCTGAAGCC | mutagenic QC primer to |
| 100 | CAGAG | make aav8 Q467E |
| SED ID NO: | cctcggacccttcGACggactcgacaagg | QC primer for AAV8 |
| 101 | | N57D |
| SED ID NO: | tacaagcaaatctccGACgggacatcgggaggag | QC primer for AAV8 |
| 102 | | N263D |
| SED ID NO: | ctacctaacactcaacGACggtagtcaggccgtg | QC primer for AAV8 |
| 103 | | N385D |
| SED ID NO: | ctgggaccaaataccatctgGATggaagaaattcattggct | QC primer for AAV8 |
| 104 | aatc | N514D |
| SED ID NO: | gagcgtttnttcccagtGACgggatcctgattntggc | QC primer for AAV8 |
| 105 | | N540D |
| SED ID NO: | ccttgtcgagtccgtcgaagggtccgagg | QC primer for AAV8 |
| 106 | | N57D |
| SED ID NO: | ctcctcccgatgtcccgtcggagatttgcttgta | QC primer for AAV8 |
| 107 | | N263D |
| SED ID NO: | cacggcctgactaccgtcgttgagtgttaggtag | QC primer for AAV8 |
| 108 | | N385D |
| SED ID NO: | gattagccaatgaatttcttccatccagatggtatttggtcccag | QC primer for AAV8 |
| 109 | | N514D |
| SED ID NO: | gccaaaaatcaggatcccgtcactgggaaaaaaacgctc | QC primer for AAV8 |
| 110 | | N540D |

B. Results

AAV8 shows substantial charge heterogeneity in its capsid proteins

To qualitatively assess the presence of post-translational modifications on the AAV8 vector capsid that could affect vector performance, we analyzed AAV8 total capsid protein purified by iodixanol gradient both by 1D and 2D gel electrophoresis. In a 1D reducing sodium dodecyl sulfate SDS gel, VP1, VP2, and VP3 resolved as single bands at the appropriate molecular weights (FIG. 1B) (Rose J A, et al. J Vrol 1971; 8(5):766-70). When further evaluated by 2D gel electrophoresis, which separates proteins based on charge (FIG. 1C), each of the capsid proteins additionally resolved as a series of distinct spots with different isoelectric points (pIs) ranging from pH 6.3 to >7.0 dependent on the VP isoform (FIG. 1D). Individual spots on each VP were separated by discrete intervals of 0.1 pI units as measured as migration relative to the carbonic anhydrase isoform internal isoelectric point standards, suggesting a single residue charge change. The presence of these isoforms suggests that each VP has the potential to undergo many modifications, thereby causing them to migrate differently under isoelectric focusing.

Deamidation, in which a fraction of (typically asparagine) side-chain amide groups are converted to carboxylic acid (FIG. 1A), is a common source of charge heterogeneity in protein preparations. To determine if deamidation could be responsible for the distinct population of VP charge isoforms, we mutated two AAV8 asparagine residues individually to aspartate. These capsid mutations should shift the charge by an amount equivalent to the complete deamidation of a single additional asparagine residue. 2D gel analysis of the mutants indicates the major spots for VP1, VP2, and VP3 shifted one spot location more acidic (0.1 pH units) than the equivalent spots in wild-type (wt) AAV8 (FIG. 1E-FIG. 1G). The magnitude of this shift is equivalent to the observed spacing between the wt VP charge isoforms. Thus, the 2D gel patterning of AAV capsid proteins is consistent with multi-site deamidation.

Spontaneous Deamidation Occurs on the AAV8 Vector Capsid

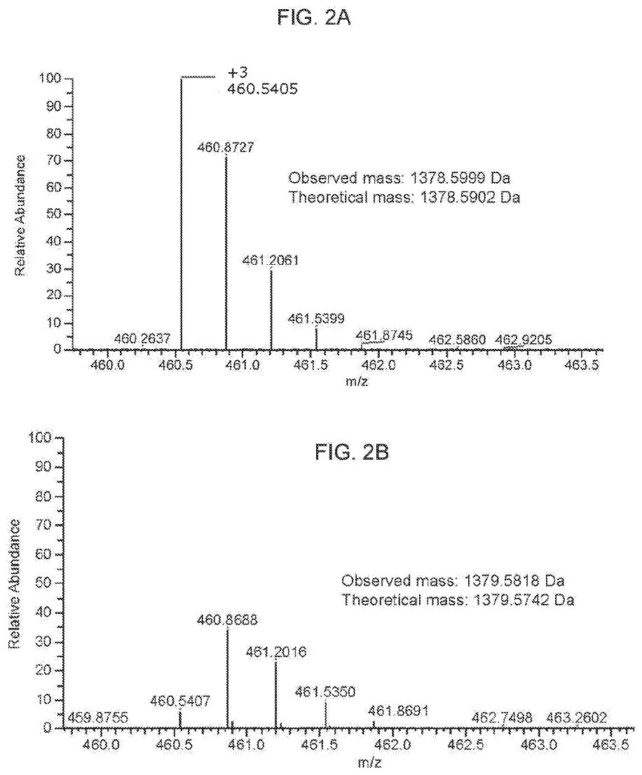

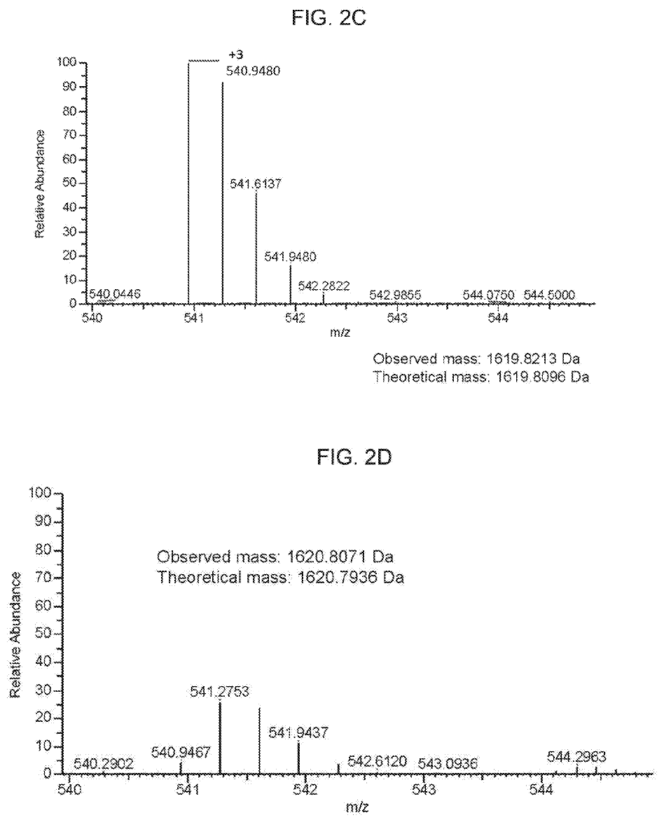

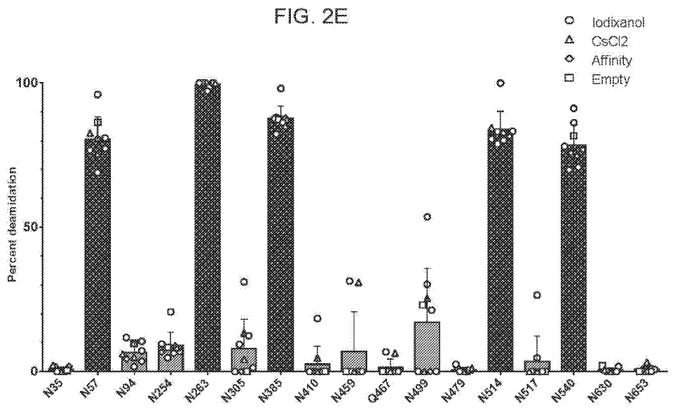

To identify modifications responsible for the discrete spotting pattern for each capsid protein, we analyzed a panel of AAV8 vectors by mass spectrometry. Coverage of the AAV8 capsid protein averaged >95% of the total VP sequence (data not shown). We detected extensive deamidation of a subset of asparagine and glutamine residues by mass spectroscopy, which showed an increase of ˜1 Da in the observed mass of the individual peptides as compared to predicted values based on the sequence encoded by the DNA; we observed this pattern of deamidation in all preparations of AAV8 vectors (FIG. 2A-FIG. 2D).

To evaluate the global heterogeneity of deamidation between commonly used purification methods and to examine deamidation in the VP1 and VP2 unique regions, we selected nine lots of AAV8 produced by triple transfection in 293 cells and purified them by either cesium chloride gradient, iodixanol gradient, or affinity chromatography. Vectors also varied with respect to promoters and transgene cassettes. To determine if the presence of the vector genome had an impact on deamidation, we also evaluated an AAV8 prep produced by triple transfection in 293 cells in the absence of cis plasmid (producing empty capsids only) and purified by iodixanol gradient.

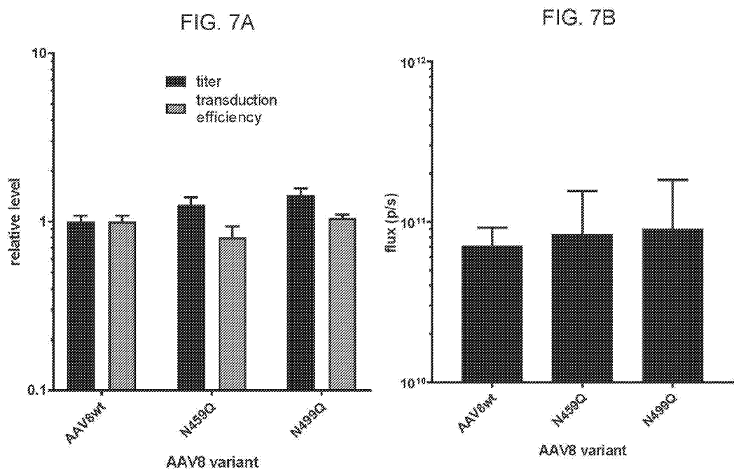

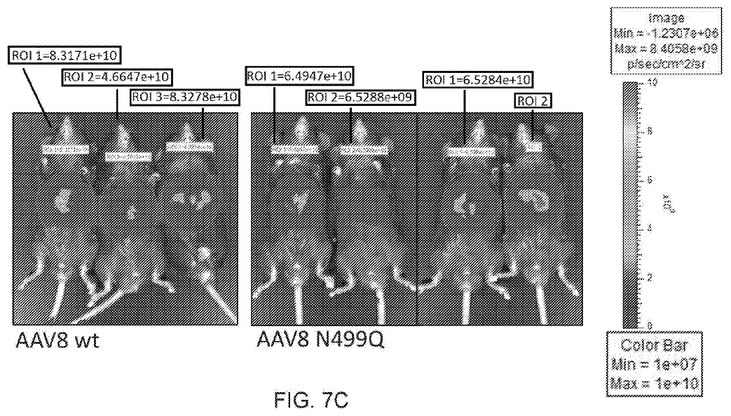

A wide range of deamidation was present across asparagine and glutamine residues of the AAV8 capsid, ranging from undetectable to over 99% of individual amino acids being deamidated (FIG. 2E). The highest levels of deamidation (>75%) occurred at asparagine residues where the N+1 residue was glycine (i.e., NG pairs) (Table 1). We detected lower levels of deamidation (i.e., up to 17%) at additional asparagine residues where the N+1 was not glycine. The average deamidation for asparagines was largely consistent between preps. We also detected deamidation at glutamine residues but at a lower frequency than at asparagines; the highest percent we observed was <2% at Q467 (FIG. 7). This observation was inconsistent across preparations (data not shown). We observed the greatest preparation-to-preparation differences at residue N499 (N+1 residue is asparagine), with values ranging from <1% to over 50% deamidation. Regardless, the variations we observed in deamidation between preparations of vector did not appear to be related to purification method, transgene identity, or the presence of vector genome, suggesting that these factors do not impact deamidation rates.

TABLE 1
Characteristics of AAV8 deamidated residues of interest.
Asterisks represent residues selected for further analysis.

| | N + 1 | Structural | Structural | Average | % Temperature |
|---|---|---|---|---|---|
| | residue | topology | motif | deamidation | factor (Å∧2) |
| N35 | Q | N/A | N/A | 1 | N/A |
| N57 | G | N/A | N/A | 80 | N/A |
| N94 | H | N/A | N/A | 7 | N/A |
| N254* | N | Surface exposed | Not assigned | 9 | 35 |
| N255* | H | Surface exposed | Not assigned | N/A | 42 |
| N263 | G | Surface exposed | HVR I | 99 | 51 |
| N305 | N | Buried | Alpha helix | 8 | 33 |
| N385 | G | Surface exposed | HVR III | 88 | 41 |
| N410 | N | Buried | Not assigned | 3 | 33 |
| N459 | T | Surface exposed | HVR IV | 7 | 65 |
| N499 | N | Surface exposed | HVR V | 17 | 45 |
| N514* | G | Surface exposed | HVR V | 84 | 36 |
| N517* | S | Surface exposed | HVR V | 4 | 40 |
| N540* | G | Buried | HVR VII | 79 | 40 |
| N630* | F | Buried | Not assigned | 1 | 32 |
| N653 | T | Surface exposed | HI loop | 1 | 35 |

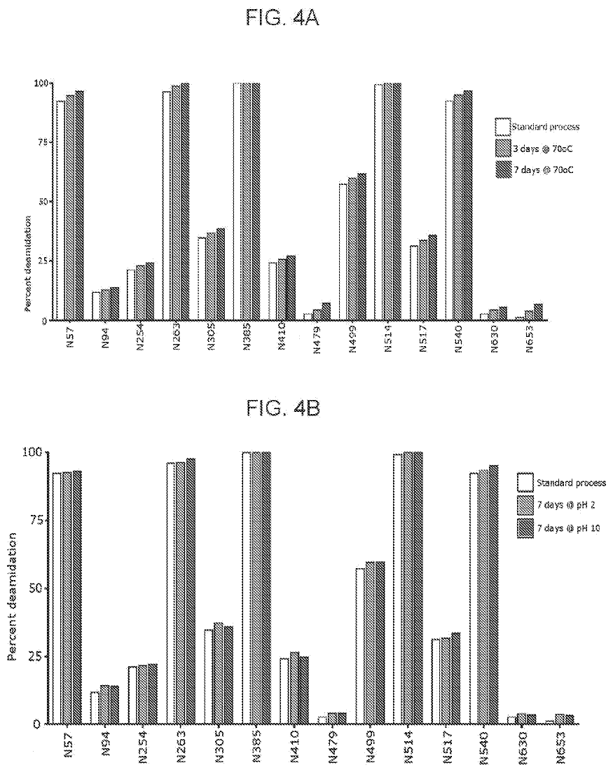

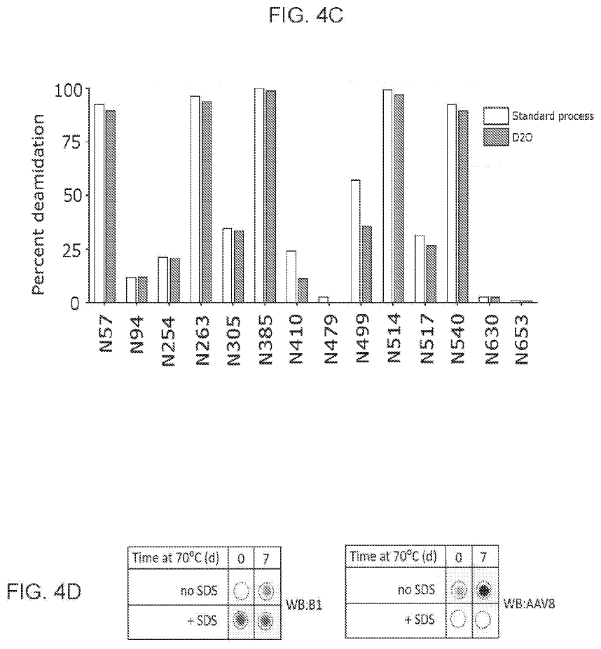

Next, we ran a series of experiments to determine if sample handling contributed to the observed levels of deamidation in AAV8. Extreme temperature (70° C. for 7 days) or pH (pH 2 or pH 10 for 7 days) did not significantly induce additional deamidation in the AAV8 capsid (FIG. 4A and FIG. 4B). Given this resistance, we reason that it was unlikely that the deamidation observed occurred only in the purification phase, which was shorter and relatively mild in comparison. We attempted to perform mass spectrometry analysis on unpurified vector to determine the extent of deamidation before and after purification, but were unsuccessful. Likewise, heavy water controls indicate that processing specific to our mass spectrometry workflow do not contribute additional deamidation events (FIG. 4C).

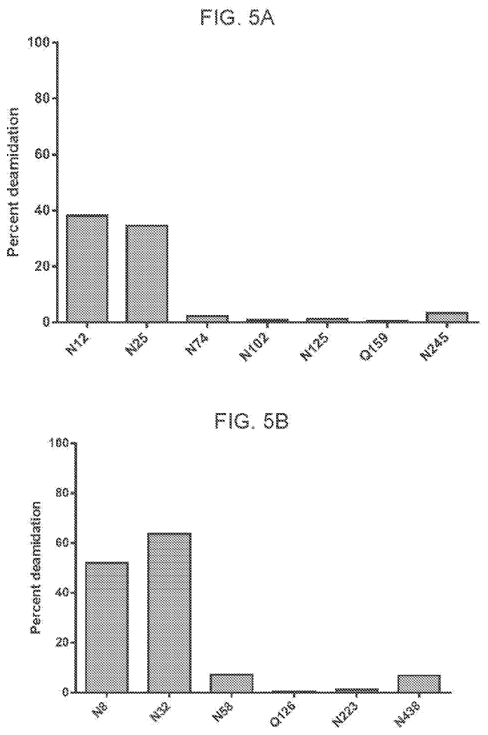

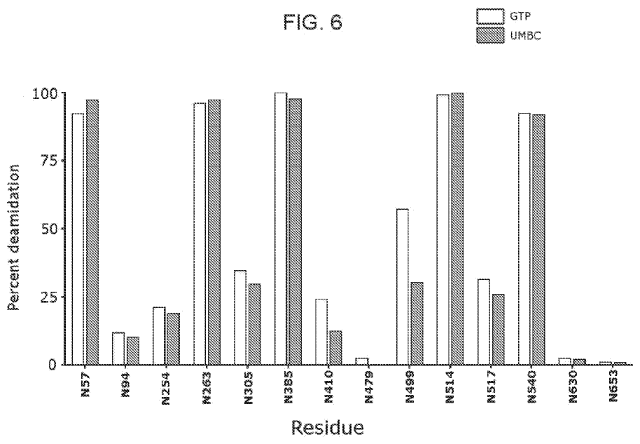

To validate our mass spectrometry workflow, we examined two recombinant proteins that have been evaluated previously for deamidation; our findings (FIG. 5A and FIG. 5B) agree with the published results [Henderson, L E, Henriksson, D, and Nyman, P O (1976). Primary structure of human carbonic anhydrase C. The Journal of biological chemistry 251: 5457-5463 and Carvalho, R N, Solstad, T, Bjorgo, E, Barroso, J F, and Flatmark, T (2003). Deamidations in recombinant human phenylalanine hydroxylase. Identification of labile asparagine residues and functional characterization of Asn-->Asp mutant forms. The Journal of biological chemistry 278: 15142-1515]. Additionally, we engaged a secondary institution to evaluate our raw data from AAV8. This independent analysis identified the same sites as deamidated, with minimal variation in the extent of modification at each site attributable to software-to-software variations in peak detection and area calculation (FIG. 6).

Structural Topology, Temperature Factor, and the Identity of the N+1 Amino Acid Contribute to Deamidation Frequency

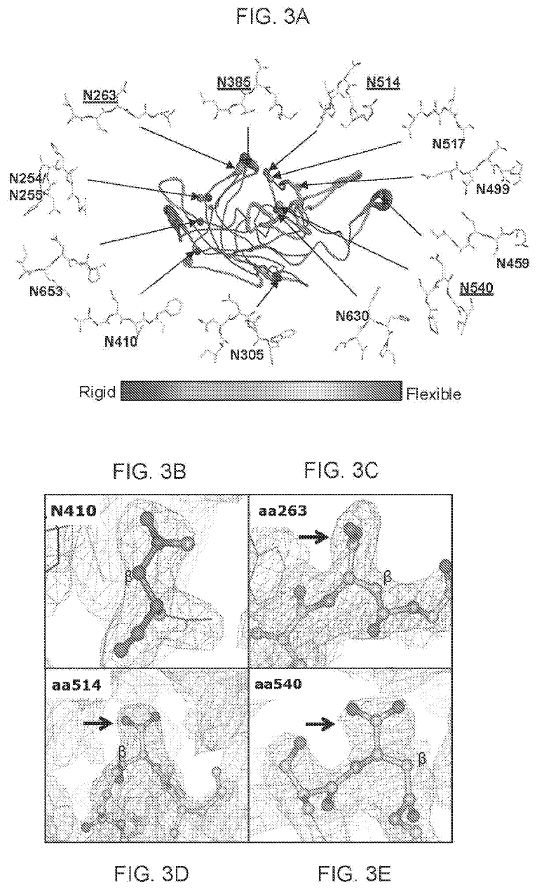

As the structure of AAV8 has been solved and published (PDB identifier: 2QA0) (Nam H J, et al. J Virol 2011; 85(22):11791-99), we next examined the AAV8 capsid structure for evidence of favorable conditions for non-enzymatic deamidation and to correlate percent deamidation with established structural features (Nam H J, et al. J Virol 2007; 81(22):12260-71). We focused on asparagine residues exclusively, as the factors influencing asparagine deamidation are better characterized in the literature and asparagine deamidation events are far more common than glutamine deamidation events (Robinson, N E, and Robinson, AB (2001). Molecular clocks. Proc Natl Acad Sci USA 98: 944-949). We also determined the temperature (or B) factor for each of these residues from the AAV8 crystal structure; temperature factor is a measure of the displacement of an atom from its mean position, with higher values indicating a larger displacement, higher thermal vibration, and therefore increased flexibility (Parthasarathy S and Murphy M R. Protein Science: A Publication of the Protein Society 1997; 6:2561-7). The majority of asparagines of interest were located in or near the surface-exposed HVRs (Table 1), which are structurally favorable for deamidation and provide a solvent-exposed environment (Govindasamy L, et al. J Virol 2013; 87(20):11187-99). We found that residues located in these flexible loop regions were, on average, more frequently deamidated than residues in less flexible regions such as beta strands and alpha helices. For example, the NG residue at position N263 is part of HVR I, has a high temperature factor, and was >98% deamidated on average (FIG. 7A and FIG. 6, Table 1). N514, which was deamidated ˜85% of the time (FIG. 3 and FIG. 6, Table 1), is also in an HVR (HVR V) with an N+1 glycine; however, the local temperature factor is relatively low in comparison to that of N263 due to its interaction with residues on other VP monomers at the three-fold axis. Less-favorable +1 residues and lower local temperature factors correlated with lower deamidation, even for HVR residues. For example, N517 was on average only 4% deamidated (Table 1); this residue has an equivalent temperature factor to the highly deamidated N514, but its N+1 residue is a serine, decreasing the likelihood of deamidation events due to steric hindrance. This demonstrates that a number of factors cumulatively determine the extent of deamidation at a given capsid position, although the identity of the +1 residue is apparently the most influential factor.

To test the role of the +1 residue in asparagine deamidation, we generated mutant vectors in which AAV8 NG sites were individually mutated at the +1 position to either alanine or serine. Model peptide studies indicate that NG peptides deamidate with a half-life as short as 1 day, whereas NA or NS peptides typically deamidate 25- or 16-fold more slowly, respectively (Robinson N E and Robinson A B. Proc Natl Acad Sci USA. 2001; 98(8):4367-72). Mass spectrometry analysis of the vector mutants confirmed the central role of the +1 site in determining the extent of vector deamidation. NG sites in this set (>80% deamidation in wt) showed selective stabilization of the adjacent asparagine when the +1 site was changed to alanine (<5% deamidation) or serine (<14% deamidation) (Table 2).

TABLE 2
Extent of deamidation (%) at five AAV8 NG sites in wt
and six + 1 site mutants.

| position\ | WT | | | | | | |
|---|---|---|---|---|---|---|---|
| variant | (average) | G58S | G58A | G264A | G386S | G386A | G515A |
| N57 | 81.8 | 8.4 | 1.9 | 89.7 | 89.7 | 91.6 | 93.6 |
| N263 | 99.3 | 98.2 | 98.9 | 4.8 | 100.0 | 94.5 | 97.2 |
| N385 | 89.1 | 96.3 | 94.8 | 97.1 | 13.5 | 2.5 | 97.0 |
| N514 | 85.2 | 100.0 | 98.0 | 98.8 | 100.0 | 100.0 | 2.2 |
| N540 | 84.5 | 95.0 | 92.6 | 97.9 | 96.9 | 86.1 | 89.5 |

Residues that were at least partially buried and not readily exposed to solvent and/or were located in regions of low local flexibility in the intact, fully assembled AAV8 capsid had a lower frequency of deamidation compared to those located in a more favorable environment Table 1). Despite this, a few of the residues in unfavorable conditions were deamidated. For example, N630 is at least partially buried but still had a detectable degree of deamidation. For this residue, the presence of phenylalanine as the N+1 residue suggests that this region could be a novel site of non-enzymatic autoproteolytic cleavage within the AAV8 VP3 protein.

Structural Modeling of AAV8 VP3 Confirms Deamidation Events

To provide direct evidence of deamidation in the context of an assembled capsid, we evaluated the crystal structure of AAV8 (Nam H-J, et al. J Virol 2011; 85(22):11791-9). The resolution of the available crystal structure (i.e., 2.7 Å) of this serotype is not high enough to identify the terminal atoms in the R groups and, therefore, is insufficient to directly distinguish between asparagine, aspartic and isoaspartic acid residues. Other aspects of the structure of the isomer of aspartic acid that forms under these conditions provided us an opportunity to determine deamidation from the 2.7 Å structure. This analysis was based on two assumptions: 1) The predominant product of spontaneous deamidation of an asparagine is isoaspartic rather than aspartic acid, which is generated at a 3:1 ratio (Geiger T and Clarke S. J Biol Chem 1987; 262(2):785-94), and 2) an asparagine or aspartic acid can be differentiated from an isoaspartic acid because the electron density map corresponding to the R group of isoaspartic acid is shorter in length. This shorter R group is created when the beta carbon from the R group of isoaspartic acid is lost when incorporated into the main chain of the AAV8 VP3 capsid protein backbone following resolution of the succinimidyl intermediate during the deamidation reaction.

We first refined the AAV8 structure itself, generating an AAV8 capsid electron density that was not biased by the known AAV8 VP3 sequence. We then examined the refined AAV8 crystal structure for evidence of deamidation based on the presence of a shorter R group associated with isoaspartic acid (FIG. 3A-FIG. 3E). The electron density map confirmed a shorter R group for the highly deamidated N+1 glycine residues at positions 263 (FIG. 3C), 385 (not shown), 514 (FIG. 3D), and 540 (FIG. 3E) when compared to the asparagine at 410 that had no deamidation detected by mass spectrometry (FIG. 3B). The deamidation indicated by the electron density map is therefore consistent with the data generated by mass spectrometry at these sites with >75% deamidation. The resulting isoaspartic acid models were comparable to isoaspartic acid residues observed in the crystal structures of other known deamidated proteins, supporting the validity of our analysis of AAV8 (Rao F V, et al. Chem Biol. 2005; 12(1):65-76; Noguchi S, et al. Biochemistry 1995; 34(47):15583-91; Esposito L, et al. J Mol Biol 2000; 297(3):713-32). This structural analysis serves as an independent confirmation of the deamidation phenomena observed when analyzing the AAV8 capsid via mass spectrometry.

Deamidation of the AAV Capsid is not Serotype Specific

We investigated serotypes beyond AAV8 for evidence of capsid deamidation. We examined AAV9 vector preparations using 2D gel electrophoresis (FIG. 1A) and mass spectrometry (FIG. 11B), including controls for potential vector-processing effects (FIG. 11D-FIG. 11F). The pattern and extent of AAV9 deamidation was similar to that of AAV8. All four AAV9 NG sites were >85% deamidated; 13 non-NG sites were deamidated to lesser extent, with a few sites showing high lot-to-lot variability in % deamidation. Next, we applied our structural analysis workflow and refit existing AAV9 crystallographic data (FIG. 11C, Table 3). As with AAV8, isoaspartic acid fit better into the electron density of several NG sites in the AAV9 crystal structure. We extended our 2D gel analysis (data not shown) and mass spectrometry (summarized in Table 4) to five additional evolutionarily diverse serotypes (rh32.33, AAV7, AAV5, AAV4, AAV3B and AAV1). All of the capsids examined contain a similar pattern and extent of deamidation, indicating that this modification is widespread in clinically relevant AAV vectors, and is determined by similar underlying primary-sequence and structural factors.

TABLE 3
Characteristics of AAV9 deamidated residues of interest. Conserved
asparagine residues with homologous N +1 residues (in comparison to
AAV8) are denoted in italics (determined by alignment of the full-
length amino acid sequences of AAV8 and AAV9 VP1).

| | N +1 | Structural | Structural | Average % | Temperature |
|---|---|---|---|---|---|
| | residue | topology | motif | deamidation | factor (Å∧2) |
| N57 | G | N/A | N/A | 97 | N/A |
| N94 | H | N/A | N/A | 5 | N/A |
| N253 | N | Surface exposed | Not assigned | 9 | 41 |
| N254 | H | Surface exposed | Not assigned | 2 | 50 |
| N270 | D | Surface exposed | HVR I | 11 | 65 |
| N304 | N | Buried | Alpha helix | 23 | 35 |
| N329 | G | Surface exposed | HVR II | 94 | 89 |
| N409 | N | Buried | Not assigned | 9 | 36 |
| N452 | G | Surface exposed | HVR IV | 98 | 64 |
| N477 | Y | Buried | Not assigned | 2 | 33 |
| N512 | G | Surface exposed | HVR V | 89 | 48 |
| N515 | S | Surface exposed | HVR V | 3 | 47 |
| N651 | T | Buried | HI loop | 1 | 38 |
| N663 | K | Surface exposed | HI loop | 4 | 49 |
| N668 | S | Surface exposed | HI loop | 13 | 52 |
| N704 | Y | Surface exposed | HVR IX | 5 | 68 |
| N709 | N | Surface exposed | HVR IX | 5 | 55 |

TABLE 4
Extent of deamidation observed for diverse serotypes

| | | Average % | | | # of non | average |
|---|---|---|---|---|---|---|
| | | sequence | | average | NG sites | non- |
| | vector | Coverage | | NG % | observed | NG % |
| | preps | by | # of | deami- | deami- | deami- |
| serotype | analyzed | MS | NGs | dation | dated | dation |
| AAV1 | 3 | 91.4 | 4 | 95.6 | 19 | 12.9 |
| AAV3B | 1 | 89.8 | 4 | 97.0 | 9 | 9.4 |
| AAV4 | 3 | 84.7 | 4 | 96.2 | 15 | 15.3 |
| AAV5 | 1 | 88.7 | 3 | 88.7 | 11 | 15.3 |
| AAV7 | 1 | 90.9 | 4 | 92.1 | 9 | 13 |
| AAV8 | 21 | 93.4 | 5 | 90.5 | 37 | 7.4 |
| AAV9 | 7 | 90.2 | 4 | 95.5 | 26 | 5.3 |
| rh32.33 | 1 | 100 | 3 | 97.4 | 14 | 16.2 |

Deamidation Events can Affect Capsid Assembly and Transduction Efficiency

One approach to testing the functional impact of deamidation is by substituting asparagine with aspartate by genetic mutation. We generated an aspartate mutant vector encoding a luciferase reporter for each deamidated AAV8 asparagine by small-scale triple transfection of 293 cells, and titered the vectors by qPCR of DNAseI resistant genome copies (FIG. 8A). The mutations rarely affected capsid assembly relative to wtAAV8, and effects were limited to mostly buried, non-NG sites with low overall deamidation in the wt vector. Next, we assessed the mutation panel for in vitro transduction efficiency of human liver-derived Huh7 cells (FIG. 8B). Several mutants showed impaired transduction efficiency, with positions N57, N94, N263, N305, Q467, N479, and N653 exhibiting >10-fold transduction loss. We observed a similar number of sensitive sites for AAV9 (FIG. 11G and FIG. 11H). As typically only a fraction of residues at a given position are deamidated endogenously, this approach has the potential to overestimate functional loss for proteins such as capsids where the functional unit is a homomeric assembly; endogenous modification at one capsid site may be compensated for by a neighboring subunit with an intact residue. Nonetheless, we reasoned that the method could help prioritize deamidated residues for future monitoring during manufacturing or mutational stabilization. Functional data from populations of endogenously deamidating vectors will be required to place this loss-of-function mutagenesis data in the proper context.

Vector Activity Loss Through Time is Correlated with Progressive Deamidation

Given the apparently short half-life of NG deamidation, we reasoned that vector samples differing in age by as little as 1 day could show distinct deamidation profiles, thus providing an opportunity to correlate endogenous deamidation to function. Our large-scale vector preparation protocol calls for triple transfection of 293 cells followed by 5 days of incubation for vector production and 1-2 days for vector purification. To approximate this process, we prepared medium scale triple transfections (10×15 cm cell culture dishes each) of 293 cells with wt AAV8. We collected vector (2×15 cm cell culture dishes/day) at 1 day intervals for 5 days, preserving the timepoints until the end of the 5 day period by freezing vector at −80° C. Next, we assessed crude vector titer and in vitro transduction efficiency as described above. As expected, the number of assembled, DNAse-resistant genome copies increased over time (FIG. 9A). We then quickly processed crude vector for early (day 1 and 2) and late (day 5) timepoints by affinity purification and measured in vitro transduction efficiency of huh7 cells. Relative transduction efficiency of the vector dropped progressively over time (FIG. 9B). In terms of transgene expression per GC added to target cells, day 5 vector was only 40% as efficient as day 1 material. This activity drop was observed for crude material as well, indicating a change in molecular composition before purification (FIG.). We observed a similar trend in activity loss for AAV9 over 5 days, with approximately 40% reduction in vector potency (FIG. 11I-FIG. 11K).

Next we measured deamidation of the time course samples by mass spectrometry. NG site deamidation progressed substantially over every interval, with an average of 25% deamidation at day 1, and >60% of sites converted by day 5 (FIG. 9C). Non-NG site deamidation generally progressed over 5 days, although at much lower levels and with less consistency between days 2 and 5 (FIG. 9D). The data correlates endogenous vector deamidation to an early timepoint decay in specific activity, and highlights a potential opportunity to capture more active vector by shortening the production cycle or finding capsid mutations that stabilize asparagines.

We note that the material used for mass spectrometry analysis in FIG. 2A-FIG. 2E was at least 7 days post-transfection, due to an additional 2 days for purification. The higher NG site deamidation in these samples (>80%) indicates that deamidation likely continues after the period of expression and during the recovery and purification processes at approximately the same rates until NG sites are completely deamidated or the vector sample is frozen. Thus deamidation is largely determined by the age of the vector and is not a process that is exclusive to or caused by the recovery and purification process. The much lower deamidation values in the day 1 material vs the day 5 material (both affinity purified) underscore this point.

Stabilizing NG Asparagines can Improve Vector Performance

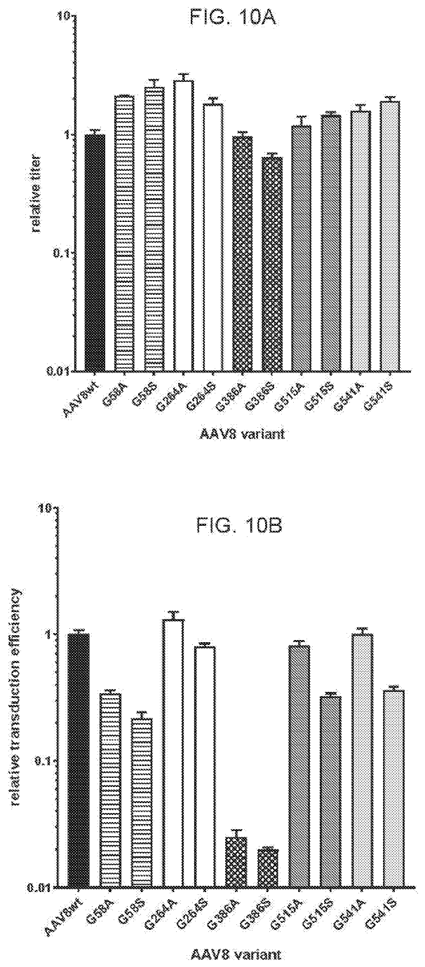

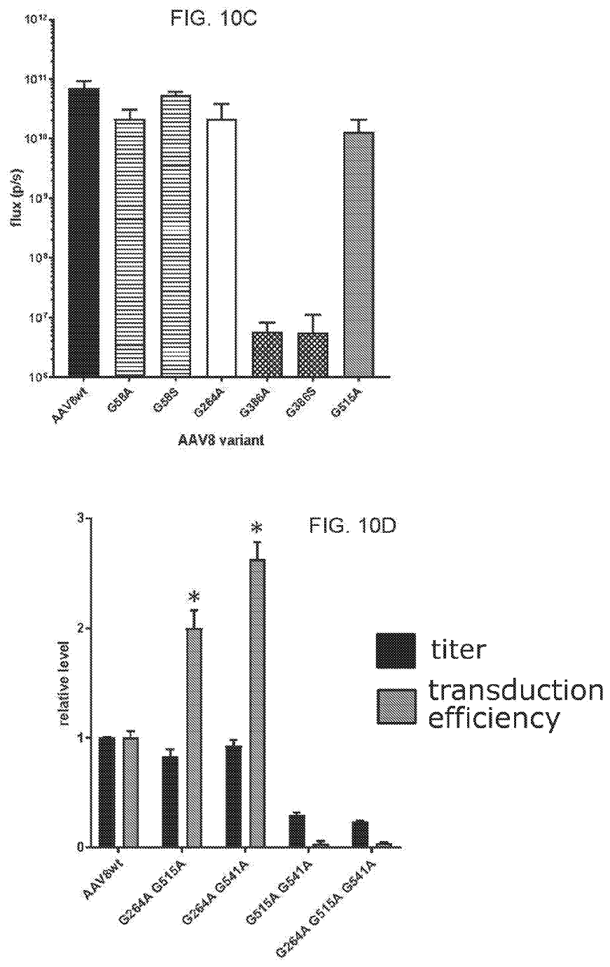

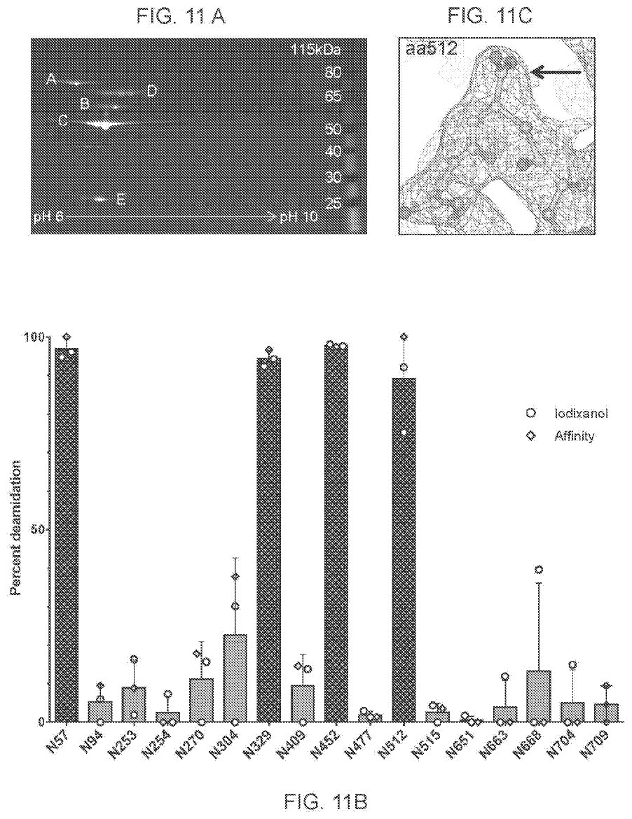

Given the correlation between vector NG deamidation and transduction efficiency loss, we reasoned that stabilizing NG amides by +1 site mutagenesis may improve vector function. We produced vector in small scale for AAV8 NG site mutants in which each +1 residue was individually converted to alanine or serine. Single+1 mutants were well tolerated in terms of vector assembly (FIG. 10A) and transduction efficiency (FIG. 10B). G386 substitutions, located near a previously defined “dead zone” on the capsid surface (Aydemir F, et al. J Virol July 2016; 90(16):7196-204), were defective for in vitro transduction. The loss of function for G386 mutants could indicate a preference for a deamidated asparagine at N385. Alternatively, the additional sidechain bulk at the +1 position may have a negative impact on function that is independent of amide-group stabilization. No single-site mutants significantly improved in vitro transduction, in spite of dramatic stabilization of their neighboring asparagines (Table 2). Because in vitro and in vivo transduction activities can be discordant, we tested a subset of the single-site+1 mutants for liver transduction in C57BL/6 mice. We performed intravenous tail vein injection (n=3 to 5) and examined luciferase expression by imaging weekly for 2 weeks (FIG. 10C). In vivo and in vitro transduction data were in agreement to within the associated errors of each assay (i.e., within the error range). G386 substitutions were defective for transduction, while +1 site mutations at other positions were largely tolerated, transducing liver at levels equivalent to but not exceeding wtAAV8.

Because stabilizing the amide at any one NG site may be necessary but not sufficient for functional restoration, we next evaluated vector variants with combinations of +1 site alanine substitutions. We recombined all 3 AAV8 NG sites for which the +1 alanine was highly functional (N263, N514, and N540). Some combinations, including the triple mutant G264A/G515A/G541A, assembled poorly and were dysfunctional for transduction. However, both pairwise combinations involving N263 (G246A/G515A and G264A/G541A) improved in vitro transduction efficiency (2.0- and 2.6-fold over wtAAV8, respectively) with no loss of titer (FIG. 10D). Because these mutations introduce at least two changes (N-amide stabilization and a +1 residue side chain substitution) these data do not conclusively link NG deamidation to functional loss. However, the data are consistent with the model established in the timecourse study in which NG site deamidation can impact in vitro transduction efficiency.

Functional Asparagine Substitutions Improve Lot-to-Lot Reproducibility in Vector Manufacturing

Another potentially problematic aspect of the vector deamidation profiles we report is the high lot-to-lot variability in deamidation at some positions. For wtAAV8, this variability is most pronounced for N459 (observed deamidation ranging from 0% to 31%) and N499 (observed deamidation ranging from 0% to 53%). Variability in post-translational modifications is typically defacto avoided during biologics development, either by avoiding clones altogether that exhibit this variability, carefully monitoring and controlling production strains and conditions, or by protein engineering of the affected candidate.

As we were unable to determine the production or processing factors contributing to N459 and N499 deamidation variability (FIG. 2E), we sought functional amino acid substitutions at these positions. We first evaluated small scale vector preparations for conservative substitutions to glutamine at each position individually. Both N459Q and N499Q were assembled efficiently into vector, and were equivalent to the wtAAV8 reference for in vitro transduction efficiency (FIG. 7A). Next, we produced the mutants in large scale and performed mass spectrometry. Consistent with our observations of extremely rare glutamine deamidation, we observed selective and complete stabilization of the glutamine amides at positions 459 or 499 in these mutants (data not shown). We evaluated these mutant lots in vivo as above for liver transduction after tail vein injection in C57BL/6 mice (FIG. 7B and FIG. 7C). The wtAAV8 vector lot used as a control in this experiment was deamidated 16.8% at N499, but no deamidation was detected at N459 (data not shown). Liver transduction at day 14 for both mutants was equivalent to wtAAV8. This data demonstrates the potential for a protein engineering approach to address the molecular variability associated with deamidation in manufactured AAV vectors.

C. Discussion

We identified and evaluated non-enzymatic deamidation of asparagine and glutamine residues on the AAV8 capsid independently by 2D gel electrophoresis, mass spectrometry, de novo protein modeling, and functional studies both in vitro and in vivo. Deamidation has been shown to occur in a wide variety of proteins and to significantly impact the activity of biologics, including antibody-based therapeutics (Nebija D et al. Int J Mol Sci 2014; 15(4):6399-411) and peptide-based vaccines (Verma A et al. Clin Vaccine Immunol. 2016; 23(5):396-402). Other viral proteins, such as the VP6 protein of rotavirus, have been shown by mass spectrometry to undergo deamidation events (Emslie K R et al. Funct Integr Genomics 2000; 1(1):12-24).

The context in which these deamidations occurred in AAV8 suggested that they are the result of spontaneous non-enzymatic events. Asparagine residues are known to be more extensively deamidated than glutamine residues; the amino acid downstream of the asparagine substantially influences the rate of deamidation with an N+1 of glycine (i.e., NG) being the most efficiently deamidated. We observed remarkable confirmation of the role of the N+1 amino acid in deamidation of AAV capsids in that every NG present in VP1 was deamidated at levels >75% while deamidation was never consistently >20% in any of the other asparagines or glutamines in the capsid. Virtually all NG motifs in the AAV8 and AAV9 capsids (i.e., 7/9) were also present on the surface of the capsid contained in HVR regions that are associated with high rates of conformational flexibility and thermal vibration. This is consistent with previous reports of NG motifs of other proteins that are located in regions where flexibility may be required for proper protein function and not in more ordered structures, such as alpha helices or beta sheets (Yan B X and Sun Y Q J Biol Chem 1997; 272(6):3190-4). The preference of NG motifs in surface exposed HVRs further enhances the rate of deamidation by providing solvent accessibility and conformational flexibility, thereby facilitating the formation of the succinimidyl intermediate. As predicted, less favorable environments lead to much lower rates of deamidation.

An important question regarding the biology of AAV and its use as a vector is the functional consequences of these deamidations. Mutagenesis of the capsid DNA to convert an asparagine to an aspartic acid allows for an evaluation of capsids in which all amino acids at a particular site are represented as aspartic acids. However, no easy strategy exists to use mutagenesis to prevent deamidations other than potentially mutating the N+1 residue, which is confounded by direct consequences of the second site mutation. We studied a limited number of variants in which the asparagine residue was converted to an aspartic acid by mutagenesis. Functional analysis included capsid assembly and in vitro and in vivo transduction. The most substantial effects of mutagenesis on vector function were those involving asparagines that were incompletely deamidated at baseline and were not surface exposed. What was surprising, however, was that mutagenesis of the highly deamidated asparagine at 514 to an aspartic acid did have some effect on function. This result suggests that the presence of residual amounts of the corresponding amide may influence function. This could be due in part to the presence of hydrogen bond interactions between N514 and D531 of another three-fold related VP3 monomer (identified in the wtAAV8 crystal structure) that are lost upon conversion of this residue to aspartic acid following deamidation.

A better understanding of the factors that influence the extent of deamidation in AAV vectors is important when assessing the impact of these deamidations on the development of novel therapeutics. Incubation of vectors under extreme conditions, known to markedly accelerate deamidation kinetics, had little effect. Coupled with our isotope incorporation studies, this result suggests that deamidation occurs during capsid assembly and is not an artifact of vector processing or mass spectrometry analysis. Deamidations at NG sites are unlikely to have substantive impact on vector performance, as the reaction was virtually complete in every sample that we evaluated. However, our initial functional studies suggest that residual amounts of non-deamidated asparagines can contribute to function. We are more concerned about sites where deamidation was less complete, which in most cases was also associated with sample-to-sample variation. An example is the asparagine at position 499 that showed deamidation ranging from 0% to 53% with a mean of 17%. It is possible that subtle differences in the conditions of vector production could contribute to this heterogeneity. The striking similarity in deamidation in AAV8 and AAV9 suggests this is a property of this entire family of viruses.

In summary, we discovered substantial heterogeneity in the primary amino acid structure of AAV8 and AAV9 capsid proteins. These studies potentially impact the development of AAV as vectors in several ways. First, the actual amino acid sequences of the VP proteins are not what are predicted by the corresponding DNA sequences. Second, aspects of the production method could lead to variations in deamidation and corresponding changes in vector function. Until we have a handle on the factors that influence deamidation rates at non-NG sites and a better understanding of their functional consequences it may be necessary to include deamidation in the characterization of clinical-grade AAV vectors. 2D gel electrophoresis can provide an overall assessment of net deamidation, although mass spectrometry will be necessary to assess deamidation at specific residues.

### Example 2: Deamidation AAV8 Triple Mutant (Clade E)

An AAV8 triple mutant capsid was used to generate an rAAV vector. The predicted amino acid sequence for the VP1 protein of this capsid is provided in SEQ ID NO: 9 herein and a nucleic acid sequence encoding the capsid is provided in SEQ ID NO:8. See, also, PCT application PCT/US17/27392, published as WO 2017/180854.

AAV8Triple mutant vectors were assessed for deamidation as described in Example 1 for AAV8. Highly deamidated residues are seen at N57, N384, N498, N513, N539. Deamidation of 10% to 40% is observed at N94, N254, N255 N304, N409, N516.

| AAV8 Triple mutant | | |
|---|---|---|
| Modification | | |
| SEQ ID NO: 9 | WL1938S | WL1938S |
| Enzyme | Trypsin | Chymotrypsin |
| % Coverage | 91.6 | 88.3 |
| N57 + Deamidation | 93.1 | 91.9 |
| N94 + Deamidation | 10.4 | 10.8 |
| ~N254 + Deamidation | 14.7 | 14.4 |
| ~N255 + Deamidation | 11.9 | 12.0 |
| N304 + Deamidation | 32.7 | 32.1 |
| N384 + Deamidation | 94.6 | 93.9 |
| N409 + Deamidation | 22.8 | 22.3 |
| N478 + Deamidation | 2.5 | 2.5 |
| ~N498 + Deamidation | 54.1 | 52.7 |
| N513 + Deamidation | 93.8 | 93.0 |
| N516 + Deamidation | 29.6 | 29.6 |
| N539 + Deamidation | 87.4 | 88.4 |
| N629 + Deamidation | 2.5 | 2.4 |
| N652 + Deamidation | 1.1 | 1.1 |
| S149 + Phosphorylation | 43.9 | 41.7 |
| S153 + Phosphorylation | 62.9 | 61.4 |
| M212 + Oxidation | 94.1 | 95.9 |
| M404 + Oxidation | 11.1 | 10.7 |
| M436 + Oxidation | 15.5 | 15.8 |
| M472 + Oxidation | 2.5 | 2.6 |
| W479 + Oxidation | 1.9 | 1.9 |
| W504 + Oxidation | 1.0 | 1.0 |
| M525 + Oxidation | 42.6 | 44.3 |
| M636 + Oxidation | 11.4 | 11.7 |
| W696 + Oxidation | 0.4 | 0.4 |

### Example 3: Further Deamidation Studies

Illustrative vectors were assessed for deamidation as described in Example 1 for AAV8 and AAV9. AAV1 falls within Clade A, AAV7 falls within Clade D, while AAV3B, AAV5, AAVrh32/33, and AAV4 are outside any of the clades A-F.

A. AAV1 Deamidation

AAV1 vectors were assessed for deamidation as described in Example 1 for AAV8 and AAV9. The results show that the vectors contain four amino acids which are highly deamidated (N57, N383, N512, and N718), based on the numbering of the primary sequence of the AAV1 VP1 reproduced in SEQ ID NO: 1.

B. AAV3B Deamidation

AAV3B vectors were assessed for deamidation as described in Example 1 for AAV8 and AAV9. High levels of deamidation are observed at four asparagine residue, N57, N382, N512, and N718, with reference to the numbering of AAV3B. These numbers are based on the AAV3B VP1 reproduced in SEQ ID NO: 2.

C. AAV5 Deamidation

AAV5 vectors were assessed for deamidation as described in Example 1 for AAV8 and AAV9. High levels of deamidation are observed at residues N56, N347, N347, and N509. Deamidation at about 1% to about 35% are observed for the position: N34, N112, N213, N243, N292, N325, N400, Q421, N442, N459, and N691. These numbers are based on the AAV5 VP1 reproduced in SEQ ID NO: 3.

| AAV5 Modification | | | | | | | |
|---|---|---|---|---|---|---|---|
| Enzyme | | Trypsin | Trypsin | Trypsin | Trypsin | Trypsin | Trypsin |
| % Coverage | N + 1 | 88.7 | 89.2 | 81.4 | 88.8 | 91.7 | 82.9 |
| N34 + Deamidation | Q | 7.6 | | | | | |
| N56 + Deamidation | G | 99.9 | 87.3 | 84.9 | 88.3 | 82.8 | 87.9 |
| ~N79 + Deamidation | E | | | | 0.3 | | |
| ~N93 + Deamidation | H | | 6.3 | 5.8 | 5.5 | 7.7 | 2.9 |
| N112 + Deamidation | L | 2.3 | | | | | |
| ~N213 + Deamidation | A | 16.5 | | | | | |
| ~N243 + Deamidation | N | 32.8 | | | | 24.8 | |
| ~N259 + Deamidation | A | | | | | 2.7 | |
| ~N292 + Deamidation | N | 27.6 | | | | 27.0 | |
| N325 + Deamidation | N | 9.9 | | | | | |
| N347 + Deamidation | G | 81.1 | 94.2 | 91.7 | 87.2 | 88.1 | 85.7 |
| ~N400 + Deamidation | N | 5.4 | 3.3 | 2.4 | 4.8 | 3.3 | 3.3 |
| ~Q421 + Deamidation | N | 7.0 | | | | | |
| ~N442 + Deamidation | N | 24.1 | | | | | |
| ~N459 + Deamidation | T | 12.5 | | | | | |
| ~N509 + Deamidation | G | 85.1 | 92.6 | 89.9 | 98.0 | 92.0 | 94.0 |
| ~N572 + Deamidation | N | | 0.9 | 4.1 | 2.5 | 0.1 | 2.3 |
| ~N691 + Deamidation | N | 23.1 | | 13.3 | 4.2 | 0.4 | 0.5 |

D. AAV7 Deamidation

AAV7 vectors were assessed for deamidation as described in Example 1 for AAV8 and AAV9. High levels of deamidation are observed at N41, N57, N384, and N514. Deamidation at rates of 1% to 25% are observed at N66, N224, N228, N304, N499, N517, N705, and N736. These numbers are based on the AAV7 VP1 reproduced in SEQ ID NO: 4.

| AAV7 Modification | WL1839S |
|---|---|
| Enzyme | Trypsin |
| % Coverage | 90.9 |
| N41 + Deamidation | 95.2 |
| ~N57 + Deamidation | 93.9 |
| N66 + Deamidation | 17.5 |
| ~N224 + Deamidation | 12.8 |
| N228 + Deamidation | 1.8 |
| ~N304 + Deamidation | 26.3 |
| ~N384 + Deamidation | 88.1 |
| N479 + Deamidation | 0.3 |
| ~N499 + Deamidation | 20.2 |
| N514 + Deamidation | 91.1 |
| ~N517 + Deamidation | 11.9 |
| N705 + Deamidation | 10.3 |
| N736 + Deamidation | 16.0 |

E. AAVrh32.33 Deamidation

AAVrh32.33 vectors were assessed for deamidation as described in Example 1 for AAV8 and AAV9. High levels of deamidation are observed at positions N57, N264, N292, N318. Deamidation between 1 to 45% are observed at positions N14, N113, Q210, N247, Q310, N383, N400, N470, N510 and N701. These number are based on the rh32.33 AAV VP1 reproduced in SEQ ID NO: 5.

| AAVrh32.33 | |
|---|---|
| Modification | WL1408S |
| Enzyme | Trypsin |
| % Coverage | 100 |
| N14 + Deamidation | 3.0 |
| N57 + Deamidation | 100.0 |
| N113 + Deamidation | 1.1 |
| Q210 + Deamidation | 14.8 |
| N247 + Deamidation | 31.1 |
| ~N264 + Deamidation | 100.0 |
| ~N292 + Deamidation | 50.2 |
| Q310 + Deamidation | 5.4 |
| ~N318 + Deamidation | 92.2 |
| N383 + Deamidation | 2.1 |
| ~N400 + Deamidation | 39.7 |
| ~Q449 + Deamidation | 2.9 |
| N470 + Deamidation | 2.6 |
| N498 + Deamidation | 0.6 |
| ~N510 + Deamidation | 27.3 |
| ~N701 + Deamidation | 6.2 |
| N731 + Deamidation | 40.2 |

F. AAV4 Deamidation

AAV4 was assessed as described previously. High levels of deamidation were observed at positions 56 and 264. Other positions with high levels of deamidation may include positions 318 and 546.

| AAV4 Modification | CS1227L | CS1227L | CS1227L |
|---|---|---|---|
| Enzyme | Trypsin | Chymotrypsin | Combined* |
| % Coverage | 84.3 | 85.1 | |
| ~Q35 + Deamidation | | 0.3 | 0.3 |
| ~N56 + Deamidation | 97.2 | 96.9 | 97.0 |
| N112 + Deamidation | 11.6 | 9.8 | 10.7 |
| ~N247 + Deamidation | 28.3 | 29.4 | 28.9 |
| ~N264 + Deamidation | 97.0 | 97.3 | 97.1 |
| ~N292 + Deamidation | 27.5 | Missing ² | 27.5 |
| N318 + Deamidation | Missing ¹ | 97.0 | 97.0 |
| N358 + Deamidation | Missing ¹ | 2.1 | 2.1 |
| ~N375 + Deamidation | Missing ¹ | 12.4 | 12.4 |
| ~N401 + Deamidation | 34.9 | 29.5 | 32.2 |
| N464 + Deamidation | 34.6 | 32.2 | 33.4 |
| ~N467 + Deamidation | 7.2 | 8.7 | 7.9 |
| N471 + Deamidation | 5.9 | 7.7 | 6.8 |
| ~Q481 + Deamidation | 3.7 | | 3.7 |
| Q489 + Deamidation | 1.4 | 0.8 | 1.1 |
| N535 + Deamidation | 38.2 | Missing ² | 38.2 |
| ~N546 + Deamidation | Missing ¹ | 93.6 | 93.6 |
| ~N585 + Deamidation | Missing ¹ | 23.9 | 23.9 |
| Q606 + Deamidation | | 0.8 | 0.8 |

¹ Not covered by trypsin
² Not covered by chymotrypsin
*If residue observed in both preps, average was taken. If residue was in one prep, only that prep was used.

The trypsin and chymotrypsin preps are reported separately. However certain residues are missed by trypsin or chymotrypsin based on sequence and peptides obtained. Where the residue is observed in both preps, the deamidation is consistent, so an average shouldn't be too far off.

### Example 4: Mapping an Adeno-Associated Virus 9-Specific Neutralizing Epitope

In this study, we sought to identify neutralizing epitopes on AAV9, which has not yet been evaluated by this epitope mapping approach. Importantly, AAV9 is currently being administered intravenously in the clinic for a number of cardiac, musculoskeletal, and central nervous system indications (Bish L T, et al. Hum Gene Ther. 2008; 19(12):1359-68; Foust K D, et al. Nature Biotechnology. 2009; 27(1):59-65; Kornegay J N, et al. Molecular Therapy. 2010; 18(8):1501-8), most notably for spinal muscular atrophy (Mendell J R, et al. N Engl J Med. 2017; 377(18):1713-22). Here, we report the highest-resolution AAV-Ab complex reconstructed to date: a 4.2 Å structure of AAV9 in complex with the potent NAb PAV9.1. Through the use of serotype swapping, alanine replacement, and additional point mutations, we validated the epitope of PAV9.1 and demonstrated the ability of the resulting mutants to significantly interfere with PAV9.1 binding and neutralizing. However, this impact on both the binding and neutralizing ability of PAV9.1 was markedly reduced or not observed when we tested mutants against a panel of polyclonal samples from a variety of sources. This result suggests that although this epitope may play a role in the neutralization of AAV transduction in some circumstances, the targeted mutation of a greater breadth of neutralizing epitopes will be required to engineer a novel capsid able evade the repertoire of NAbs responsible for blocking AAV transduction.

A. Materials and Methods

1. Hybridoma Generation Balb/c mice received up to five immunizations of the AAV9 vector. We harvested and fused the splenocytes. ProMab Biotechnologies, Inc. (Richmond, Calif.) generated the clonal supernatants according to the company's standard custom mouse monoclonal antibody hybridoma development protocol. Thirty supernatants underwent screening for AAV9 reactivity by ELISA and for their ability to neutralize AAV9 by NAb assay. We obtained purified PAV9.1 mAb following screening at a concentration of 3 mg/mL.

2. AAV capsid ELISA

Corning polystyrene high bind microplates were coated with 1e9 GC/well AAV diluted in phosphate buffered saline (PBS) and kept overnight at 4° C. After discarding the coating solution, we blocked the plates with 3% bovine serum albumin (BSA) in PBS for 2 hours at room temperature followed by a triple wash of 300 μL PBS+0.05% Tween. We then incubated the hybridoma supernatant, purified mAb, serum, or plasma (diluted in 0.75% BSA in PBS) at 37° C. for 1 hour, followed by a triple wash of 300 μL PBS+0.05% Tween. Next, we detected mouse samples using 1:10,000 goat anti-mouse IgG HRP (diluted in 0.75% BSA in PBS; cat. 31430; Thermo Fisher Scientific, Waltham, Mass.) at 37° C. for 1 hour followed by a triple wash of 300 μL PBS+0.05% Tween. The human and non-human primate samples were then detected using 1:10,000 (diluted in PBS) goat anti-human IgG biotin-SP (cat. 109-065-098, Jackson ImmunoResearch Inc., West Grove, Pa.) at room temperature for 1 hour, followed by a triple wash of 300 μL PBS+0.05% Tween and 1:30,000 (diluted in PBS) unconjugated streptavidin (cat. 016-000-084, Jackson ImmunoResearch Inc., West Grove, Pa.) at room temperature for 1 hour (followed by 3× wash with 300 μL PBS+0.05% Tween). We developed all ELISAs with tetramethylbenzidine.

3. Neutralizing Antibody Assays

We performed NAb assays as previously described (Calcedo R, et al. J infect Dis. 2009; 199(3):381-90) with a few modifications. We used HEK293 cells seeded at a density of 1e5 cells/well on black-walled, clear-bottomed, poly-lysine-coated plates (cat. 08-774-256, Fisher Scientific Company, Hampton, N.H.). Using a multiplicity of infection of 90 wtAd5/cell, we utilized a working solution of 4e10GC/mL AAV9.CMV.LacZ vector to achieve a final concentration of 2e9GC/well. We measured bioluminescence with the SpectraMax M3 (Molecular Devices, Sunnyvale, Calif.), following the manufacturer's protocol. For any given sample, we defined the NAb titer as the last dilution at which AAV transduction was reduced by >50% in the presence of the sample compared to WT.AAV transduction in the presence of the naïve control. We performed HEK293 transduction experiments as described above, but withheld the neutralizing sera.

4. Fab Generation and AAV-Fab Complexing

PAV9.1 Fab (0.211 mg/mL) was generated using a Pierce Fab Preparation kit (Thermo Fisher Scientific, Waltham, Mass.) according to the manufacturer's instructions. Next, we complexed PAV9.1 Fab with the AAV9 vector at a ratio of 600 Fab:1 AAV9 capsid (or 10 Fab:1 potential binding site) at room temperature for 30 minutes.

5. Cryo-EM Sample Preparation, Data Acquisition, and Complex Reconstruction

Sample preparation: We applied 3 μL of PAV9.1-AAV9 complex to a freshly washed and glow-discharged holey carbon grid. After blotting for 3 to 4 seconds with Whatman #1 filter paper at 22° C. and 95% relative humidity, we rapidly froze the grid in liquid ethane slush using a Vitrobot Mark IV (FEI). Next, we applied a single 3 to 4 second blot with Whatman filter paper at 22° C. in 95% relative humidity. After freezing, grids were stored in liquid nitrogen. We then transferred the grids to an FEI Talos Arctica electron microscope operating at 200 kV and equipped with a Gatan K2 Summit direct electron detection camera (Gatan, Pleasanton, USA).

Data acquisition: We acquired data using the SerialEM software (Mastronarde D N. J Struct Biol. 2005; 152(1):36-51). Images were captured at a nominal magnification of 22,000× (corresponding to a calibrated pixel size of 0.944 Å) and a dose rate of 2.21 electrons/square Angstrom/sec with a de-focus range of 1.0-2.0 μm (Rohou A. and Grigorieff N. Struct Biol. 2015; 192(2):216-21). For each exposure, we recorded a 60-frame dose-fractionated movie stack in super-resolution mode for a total of 12 seconds. The movie frames were aligned using the “alignframes” program within the IMOD software package (Kremer J R, et al. J Struct Biol. 1996; 116(1):71-6).

Data collection and processing: We manually extracted all particle images from each of the micrographs and processed them using the e2boxer program available in the EMAN2 suite (Tang G, et al. J Struct Biol. 2007; 157(1):38-46). The boxed particles were then transferred into the AUTO3DEM program for cryo-reconstruction, leading to the initial low-resolution model (30 Å) based on 150 particle images (Yan X, et al. J Struct Biol. 2007; 157(1):73-82). The program adopted a random model generation procedure, and we applied strict 60 non-crystallographic symmetry axes. This low-resolution reconstructed model map was useful for determining the particle origin, conducting a full orientation, and refining the contrast transfer function of all images using AUTO3DEM. To improve the reconstructed map's quality, we applied a temperature factor correction and visualized the map in the graphics programs Coot and Chimera (Pettersen E F, et al. J Comput Chem. 2004; 25(13):1605-12; Emsley P and Cowtan K. Acta Crystallogr D Biol Crystallogr. 2004; 60(Pt 12 Pt 1):2126-32). We used a temperature factor 150-corrected map for model docking and interpretation. We extracted a total of 3,022 boxed particles from 1,100 micrographs to ultimately generate a 4.2 Å resolution reconstructed map with a Fourier shell correlation of 0.15. A VIPER database was used to generate the AAV9-60mer model while applying strict icosahedral symmetry axes (T=1) (Carrillo-Tripp M, et al. Nucleic Acids Res. 2009; 37(Database issue):D436-42). Using the FIT function in the Chimera program, we docked the 60-mer copy of AAV9 capsid into the cryo-reconstructed electron density map. This produced a correlation coefficient of 0.9. We visualized and adjusted the docked model in Coot and Chimera for accuracy. A BodyBuilder was used to generate the antibody model, which was then docked and manually adjusted into the cryo-reconstructed density using Chimera (Leem J, et al. MAbs. 2016; 8(7):1259-1268). The model was then visualized for interpretation of AAV9 and antibody-binding regions. We produced all figures using the Chimera and PyMOL programs. The RIVEM program was used to create a two-dimensional depiction of the roadmap (DeLano W L. PyMOL: An Open-Source Molecular Graphics Tool. 2002; Vol. 40:82-92). We used the RIVEM program to create a two-dimensional depiction of the roadmap (Xiao C and Rossmann M G. J Struct Biol. 2007. 158(2):182-7).

6. AAV9-PAV9.1 Mutant Trans-Plasmid Construction

We used an in-house trans-plasmid construct pAAV2/9 (AAV2 rep/AAV9 cap) for AAV9 capsid mutagenesis. All capsid mutants were constructed using a Quikchange Lightning Mutagenesis kit (Agilent, Santa Clara, Calif.) per manufacturer's instructions.

7. Vector Production

We produced the AAV9.CMV.LacZ.bGH and AAV9 mutant vectors via triple transfection in HEK293 cells followed by iodixanol gradient purification as previously described (Lock M, et al. Hum Gene Ther. 2010; 21(10):1259-71). The University of Pennsylvania Vector Core titered the vectors using quantitative PCR (qPCR) against the bGH polyA as previously described (Lock M, et al. Hum Gene Ther. 2010; 21(10):1259-71).

8. Determining EC50 of PAV9.1 mAb and Polyclonal Sera/Plasma.

We performed capsid capture ELISA with either AAV9.WT or AAV9 mutant vector as described above. We calculated the EC50 values using GraphPad Prism. Briefly, we log-transformed the PAV9.1 mAb concentration in mg/mL and plotted it on the x-axis. We defined IgG concentration in mouse plasma as 5 mg/mL (Mink J G. Serum immunoglobulin levels and immunoglobulin heterogeneity in the mouse. Diss. Erasmus MC. 1980) and in non-human primate and human serum as 10 mg/mL (Gonzalez-Quintela A, et al. Clinical and Experimental Immunology. 2008; 151(1):42-50). The plasma/serum concentration (in μg/mL) was log-transformed and plotted on the x-axis. We defined the maximum absorbance achieved with each mutant, normalized the absorbance to 100%, and plotted it on the y-axis. We then generated a dose-response curve (antibody binding) using GraphPad Prism's “log(agonist) vs. normalized response—Variable slope” function. Finally, we calculated the EC50 for PAV9.1 mAb, polyclonal serum, or polyclonal plasma.

9. Animal Studies

Our animal protocol was approved by and conducted in accordance with the standards of the University of Pennsylvania's Institutional Animal Care and Use Committee. Male C57BLJ6 mice (n=3) received intravenous injections in the tail vein of 1e11 GC/mouse AAV9.CMV.LacZ.bGH or AAV9 mutant vectors with the same transgene cassette. Animals were sacrificed 14 days after receiving vector. Each animal's organs were divided and either snap frozen on dry ice for bio-distribution or embedded in optimal cutting temperature compound and frozen for subsequent sectioning and staining for β-gal activity.

10. Bio-Distribution Analysis

We extracted DNA from tissues of interest using a QIAamp DNA Mini kit (Qiagen, Hilden, Germany). We analyzed the tissues for vector GCs by qPCR against the bGH polyadenylation signal as previously described (Chen S J, et al. Hum Gene Ther Clin Dev. 2013; 24(4):154-60).

11. β-Gal Activity Staining

Frozen sections were fixed with 0.5% glutaraldehyde in PBS for 10 minutes at 4° C. and subsequently stained for β-gal activity. After washing in PBS, we incubated the sections in 1 mg/ml X-gal (5-bromo-4-chloro-3-indolyl-β-D-galactopyranoside) in 20 mM potassium ferrocyanide, 20 mM potassium ferricyanide, 2 mM MgCl₂ in PBS (pH ˜7.3) and kept tissues overnight at 37° C. After counterstaining the sections with Nuclear Fast Red (Vector Laboratories), we dehydrated them using ethanol and xylene, followed by cover slipping.

B. Results

1. The NAb PAV9.1 is Potent and Specific for AAV9

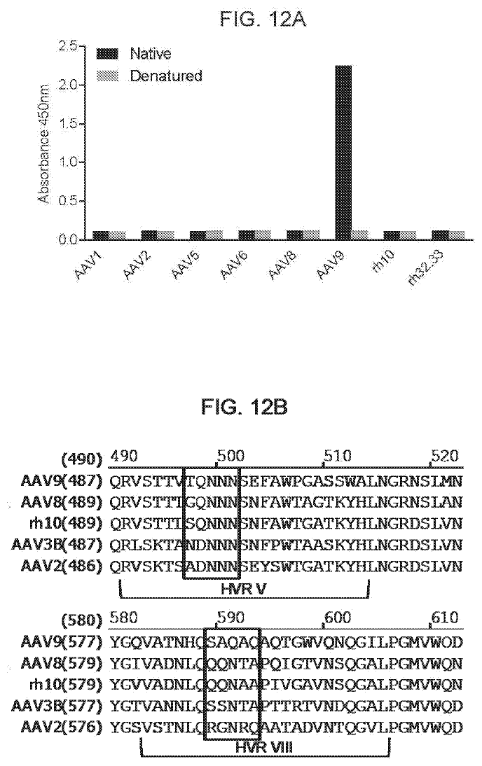

We first aimed to identify a novel, potent anti-AAV9 NAb for epitope mapping. We screened a panel of 30 hybridoma clones for AAV reactivity by an enzyme-linked immunosorbent assay (ELISA) against a number of serotypes and for AAV9 neutralization by an NAb assay. We selected the monoclonal antibody PAV9.1 from this panel due to its specificity for AAV9 (FIG. 12A). PAV9.1 recognized only intact capsid by ELISA (FIG. 12A) and did not recognize AAV by Western blot (data not shown), suggesting that PAV9.1 identifies a conformational epitope on the capsid surface. This is in contrast to the remaining clones, which more broadly bound the panel of AAVs included in the screening and also recognized AAV by Western blot (data not shown). In an NAb assay, purified PAV9.1 mAb showed an effective NAb titer of 1:163, 840, indicating that this novel anti-AAV9 antibody is a potent neutralizer of AAV9. Again, this was in contrast to the other clones screened by NAb assay, none of which were able to neutralize AAV transduction.

2. Cryo-Reconstruction of AAV9 in Complex with PAV9.1

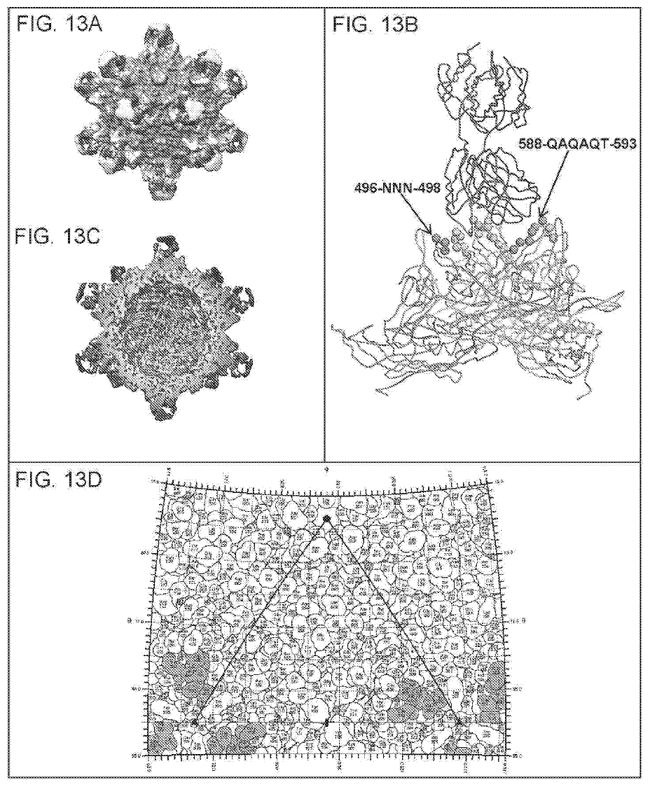

Following complexing of AAV9 with PAV9.1 antigen-binding fragments (Fab), we captured 1,100 images, boxed 3,022 particles, and generated a 4.2 Å reconstruction of the complex using AUTO3DEM. We observed Fab density extending from the three-fold axis comprised by HVRs IV, V, and VIII, and decorating the interior face of the three-fold protrusions with the Fab electron density centered perpendicularly (FIG. 13A and FIG. 13B). This region was mainly comprised of charged residues, which favor strong electrostatic interactions between three-fold related VP monomers as well as with receptors and mAbs. A single Fab molecule was bound and extended across two of the three protrusions at each three-fold axis, blocking binding of additional Fab molecules at these sites due to steric hindrance (FIG. 13C). The region of the PAV9.1 Fab complementary-determining regions (CDRs) in contact with the three-fold protrusions had an average density of 2.5 sigma levels, which is comparable to densities reported for other AAV-Fab reconstructions. We observed a PAV9.1 Fab constant region density at approximately 0.8 sigma levels, or approximately one third of the density observed for the contact region of the PAV9.1 CDRs, corresponding to a single Fab occupancy per three-fold axis. The PAV9.1 Fab CDRs directly interacted with residues 496-NNN-498 (HVR V) and 588-QAQAQT-593 (HVR VIII) (FIG. 13C and FIG. 13D). PAV9.1 binding additionally occluded residues G455 and Q456 (HVR IV), T494, Q495, and E500 (HVR V), and N583, H584, S586, and A587 (HVR VIII), which do not participate in electrostatic interactions with PAV9.1 but may provide structural stability to this region of the capsid following Fab binding (Table 3). The CDRs of the heavy chain interacted with the HVR V, whereas the CDRs of the light chain interacted with HVR VIII of the same VP3 monomer (FIG. 13C).

Table 3: PAV9.1 Fa Epitope Residues

Contact Residues

| | |
|---|---|
| HVR | Position |
| V | 496-NNN-498 |
| VII | 588-QAQAQT-593 |

Occluded Residues

| | |
|---|---|
| HVR | Position |
| IV | G455, Q456 |
| V | T494, Q495, E500 |
| VIII | N583, H584, S586, A587 |

Based on the PAV9.1 footprint (FIG. 13D, Table 3), we selected two sets of five residues for focused mutagenesis for epitope validation and escape mutant design: 586-SAQAQ-590 and 494-TQNNN-498. We chose residues 586-SAQAQ-590 because this site contains a high degree of sequence diversity (FIG. 12B). The selected motif contains residues identified by the reconstruction to be directly interacting with PAV9.1 as well as residues identified as occluded, allowing for the interrogation of the junction between bound and occluded residues. These residues have also been implicated in neutralizing epitopes for AAV1, AAV2, and AAV8, allowing for the comparison of the AAV9 epitope residues to those previously published (Tseng Y S and Agbandje-McKenna M. Front Immunol. 2014; 5:9). Finally, restricting HVR VIII mutagenesis to these five residues increased the likelihood that the capsid would tolerate larger mutations, as this motif has more limited interactions with regions contributing to capsid structural integrity. Despite PAV9.1 being specific for AAV9, the HVR V motif 496-NNN-498 identified as interacting with PAV9.1 is highly conserved between serotypes (FIG. 12B). However, unpublished phage display work (data not shown) suggested the involvement of an asparagine-rich motif in the epitope of PAV9.1; thus, we selected this motif for mutagenesis. We also added residues 494-TQ-495 to again interrogate the junction between bound and occluded residues and because they were previously implicated in AAV-Ab interactions (Tseng Y S and Agbandje-McKenna M. Front Immunol. 2014; 5:9).

3. Epitope-Based Mutations Markedly Reduce AAV9-PAV9.1 Binding

We first generated 586-SAQAQ-590 serotype swap mutants using site-directed mutagenesis. Based on the knowledge that PAV9.1 specifically recognizes AAV9 and that the amino acid sequence and structural conformation at this location varies widely between AAV serotypes, we chose full swaps with the corresponding residues from representative serotypes from Clade B (AAV2), Clade C (AAV3B), and Clade D/E (AAV8/rh10) (Table 4).

TABLE 4
Mutagenesis strategy of PAV9.1 HVR VIII epitope residues

| | | | Residue (AAV9 VPI numbering) | | | | |
|---|---|---|---|---|---|---|---|
| Vector | Serotype | Clade | 586 | 587 | 588 | 589 | 590 |
| AAV9.WT | AAV9 | F | S | A | Q | A | Q |
| AAV9.AAQAA | AAV9-like | N/A | A | A | Q | A | A |
| AAV9.QQNAA | AAV8/rh10 | D/E | Q | Q | N | A | A |
| AAV9.SSNTA | AAV3B | C | S | S | N | T | A |
| AAV9.RGNRQ | AAV2 | B | R | G | N | R | Q |
| AAV9.RGHRE | AAV2-like | N/A | R | G | H | R | E |

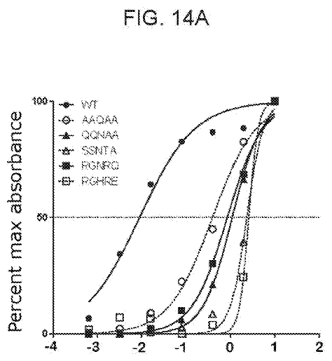

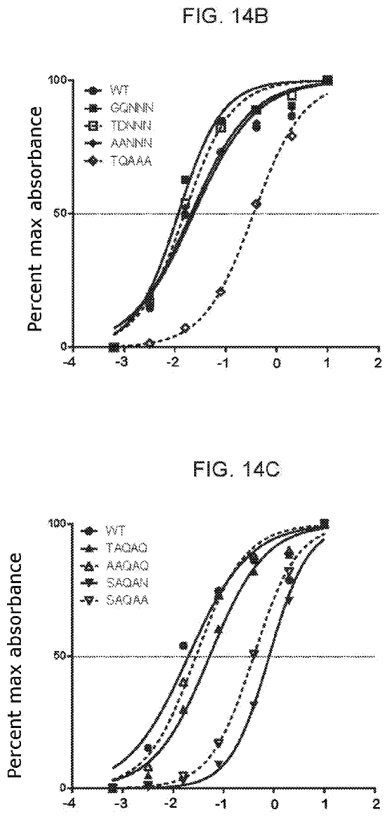

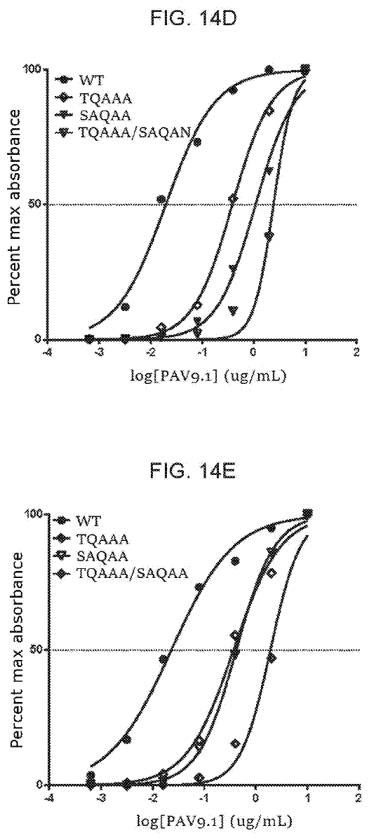

In doing so, we expected to maximize the likelihood of efficient capsid assembly while also maximizing the natural variation at this location. We generated two additional mutants, AAV9.AAQAA (more convergent than AAV9.QQNAA) and AAV9.RGHRE (more divergent than AAV9.RGNRQ), to determine (1) the minimum mutation required to disrupt PAV9.1 interactions and (2) the maximum disruption that we could introduce. AAV9.AAQAA, AAV9.QQNAA, and AAV9.SSNTA mutants produced vectors of equivalent titer to AAV9.WT; however, titers of AAV9.RGNRQ and AAV9.RGHRE were reduced two- to three-fold relative to AAV9.WT (data not shown). We determined the binding of PAV9.1 mAb to each mutant capsid compared to AAV9.WT by capture ELISA (FIG. 14A). The EC50, or the concentration of PAV9.1 mAb required to reach half-maximal binding, of PAV9.1 for each swap mutant was markedly increased (indicative of reduced capsid binding) relative to the EC50 for AAV9.WT. This result validated the epitope mapping results, indicating that residues 586-SAQAQ-590 are involved in AAV9-PAV9.1 interactions. The EC50 increases ranged from 45-fold (AAV9.AAQAA) to almost 300-fold (AAV9.RGHRE) (Table 5); the increase in EC50 directly correlated with the degree of sequence divergence from AAV9 at this location. The one exception was AAV9.RGNRQ, which shares Q590 with AAV9, potentially contributing to stronger PAV9.1 binding than that expected by sequence analysis.

TABLE 5
Summary of AAV9 capsid mutant characteristics
following in vitro evaluation

| | Fold reduction | Fold increase | Percent WT |
|---|---|---|---|
| | NAb titer | EC50 | transduction |
| WT | 1 | 1 | 100 |
| AAQAA | 16 | 45 | 27 |
| QQNAA | 128 | 124 | 53 |
| SSNTA | 512 | 264 | 58 |
| RGNRQ | 8 | 96 | 233 |
| RGHRE | 2048 | 294 | 60 |
| TQAAA | 16 | 15 | 50 |
| SAQAN | 16 | 40 | 76 |
| SAQAA | 4 | 20 | 54 |

As the S586A and Q590A mutations in AAV9.AAQAA were sufficient to disrupt PAV9.1 binding of AAV9, we next determined the minimal change required to induce this disruption. We introduced a point mutation at one of these positions either by alanine replacement or a more conservative replacement (S->T or Q->N). Mutations to either alanine or threonine at S586 did not significantly reduce PAV9.1 binding, whereas a single mutation to either alanine or asparagine at Q590 was sufficient to disrupt capsid recognition by PAV9.1 (FIG. 14C). This result indicates that position 590 is critical for PAV9.1 recognition of the AAV9 capsid.

We next interrogated the 494-TQNNN-498 motif of HVR V for its inclusion in the PAV9.1 epitope using the same mutagenic strategy: mutating sets of residues to evolutionarily conserved amino acids or alanine alone. As 496-NNN-498 is conserved across all serotypes tested, we used only alanine replacement for this stretch of residues; for 494-TQ-495, we mutated to AA as well as GQ and TD in order to represent the naturally occurring diversity at this site. Despite the specificity of PAV9.1 for AAV9 and the diversity at this location, AAV9.GQNNN, AAV9.TDNNN, and AAV9.AANNN did not increase the EC50 of PAV9.1 for AAV (FIG. 14B). This confirms the conclusion from the cryo-reconstruction map that the 494-TQ-495 site does not participate in the PAV9.1 epitope. However, the AAV9.TQAAA mutation increased the PAV9.1 EC50 15-fold, indicating that despite the fact that 496-NNN-498 is a conserved motif, it still plays an important role in the AAV9-specific binding of PAV9.1. Finally, we generated combination mutants from HVR V and minimal HVR VIII mutations (AAV9.TQAAA/SAQAN, AAV9.TQAAA/SAQAA); PAV9.1 EC50 values for these combination mutants show that the effects of changing motifs in the PAV9.1 epitope are additive (FIG. 14D and FIG. 14E).

4. Epitope-Based Mutations Modulate AAV9 Transduction

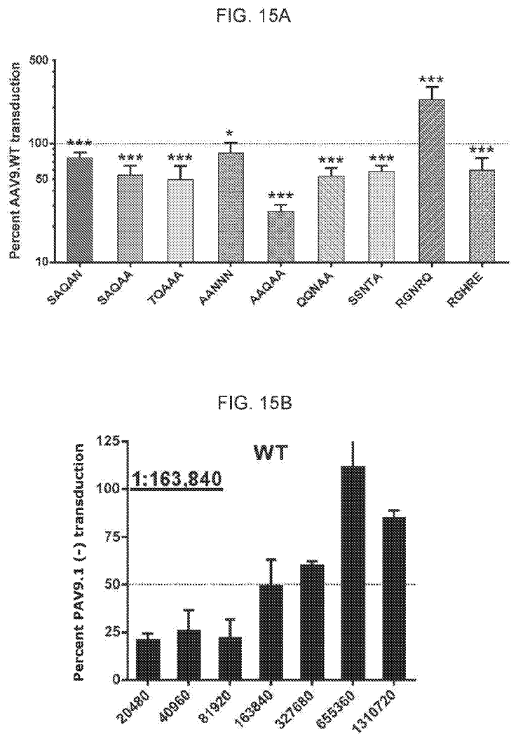

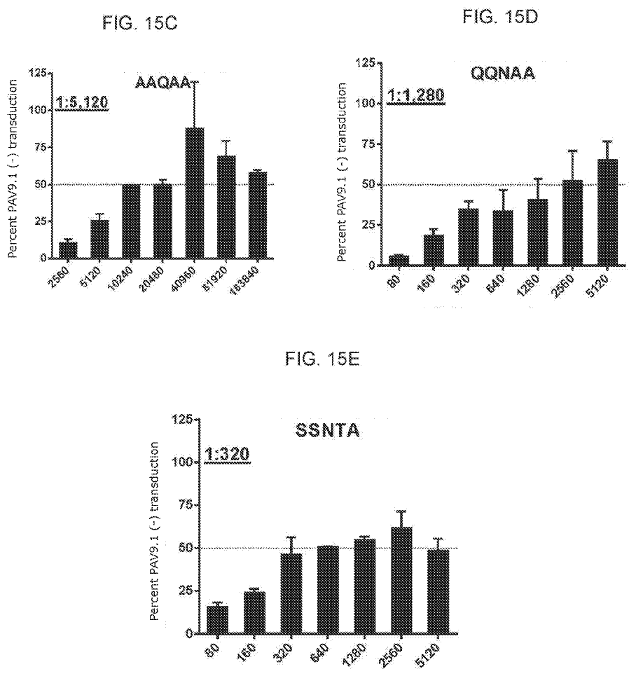

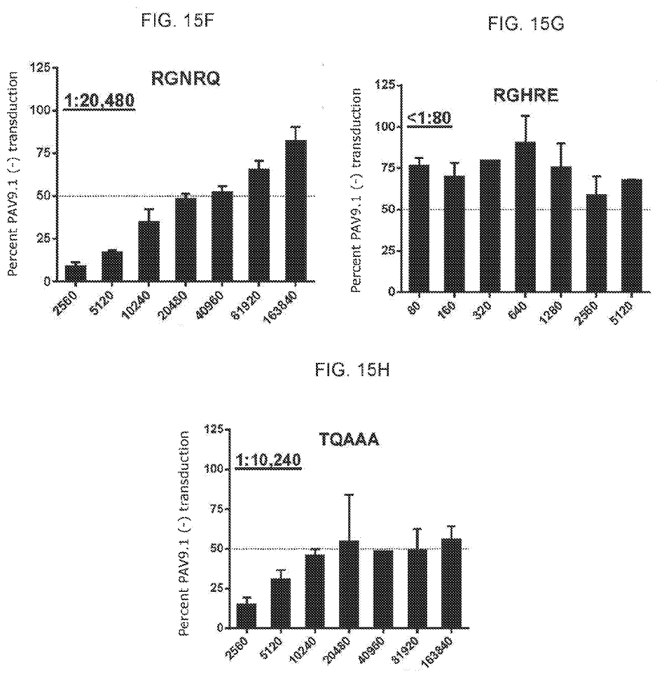

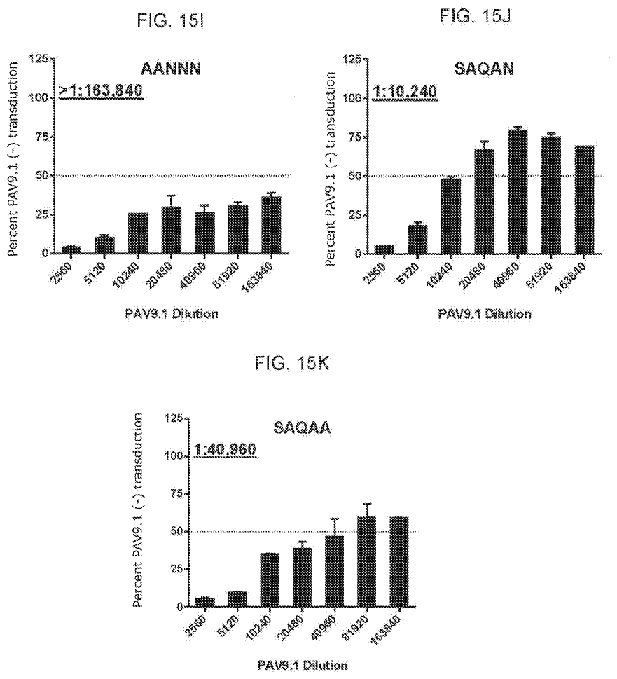

To evaluate the ability of the novel AAV9 mutants to evade NAbs while maintaining the properties of AAV9.WT, we first assessed in vitro and in vivo transduction. The majority of mutations resulting in a reduction in PAV9.1 binding also reduced the transduction efficiency in HEK293 cells, with the notable exception of AAV9.RGNRQ, which improved vector transduction by 2.3-fold (FIG. 15A). This improvement could have been due to the introduction of R586 and R589 (R585 and R588 by AAV2 VP1 numbering), two residues responsible for heparin recognition by AAV2, which performs significantly better than AAV9 in vitro in most cell lines likely due to the inclusion of these heparin-binding motifs (Ellis B L, et al. Virol J. 2013; 10(1):74) likely due to the inclusion of these heparin-binding motifs. However, AAV9.RGHRE, which shares R586 and R589 with AAV9.RGNRQ, did not display AAV2-like transduction efficiency, suggesting the involvement of other factors. AAV9.AAQAA demonstrated the greatest reduction in transduction efficiency, indicating that S586 and/or Q590 are essential residues for AAV9 transduction in vitro.

5. Epitope-Based Mutations Ablate PAV9.1 Neutralization

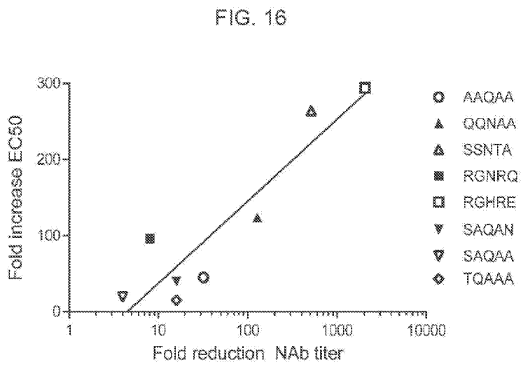

We next examined the effects of the mutations on the neutralizing titer of PAV9.1. Mutant AAV9.AANNN, which does not affect PAV9.1 binding, did not affect the neutralizing titer (FIG. 15B and FIG. 15I). However, all mutant vectors that increased the PAV9.1 EC50 reduced the effective neutralizing titer of PAV9.1. AAV9.RGHRE, which most dramatically increased the EC50 by almost 300-fold, reduced the NAb titer of PAV9.1 by at least 2,048-fold (from 1:163, 840 to <1:80, the lowest dilution tested) (FIG. 15C-FIG. 15K). Mutant vectors that increased the EC50 more modestly, such as AAV9.SAQAN, reduced the effective NAb titer of PAV9.1 to a lesser degree (FIG. 15L). Overall, we observed a strong correlation between reduction in PAV9.1 binding as measured by EC50 and reduction in effective NAb titer (FIG. 16). A notable exception was again AAV9.RGNRQ, which reduced NAb titer by only eight-fold (the second lowest reduction) despite being the fourth most effective mutant at reducing PAV9.1 binding.

6. The PAV9.1 Epitope is Important for AAV9 Liver Tropism

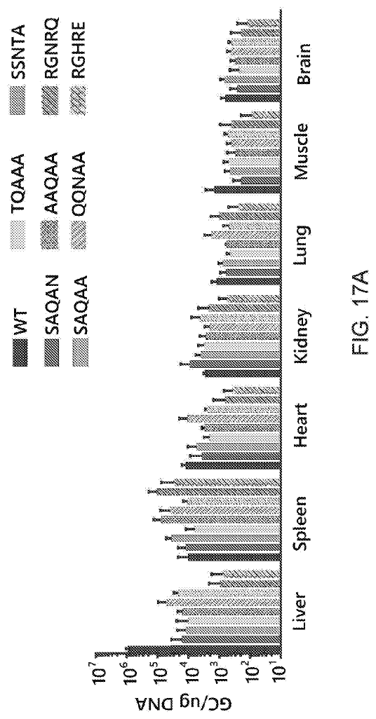

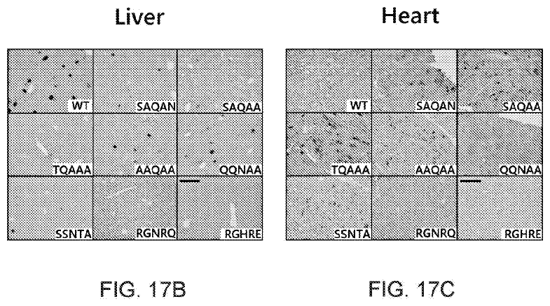

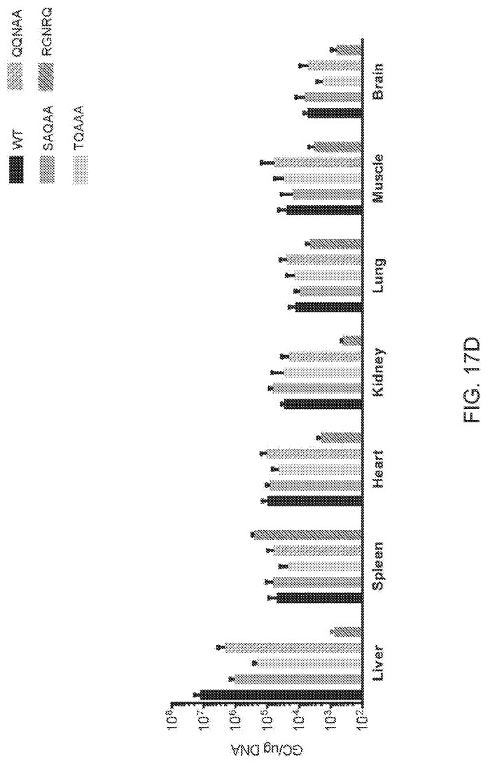

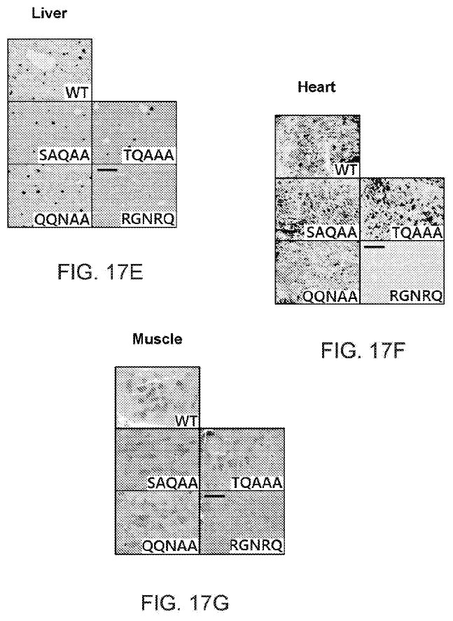

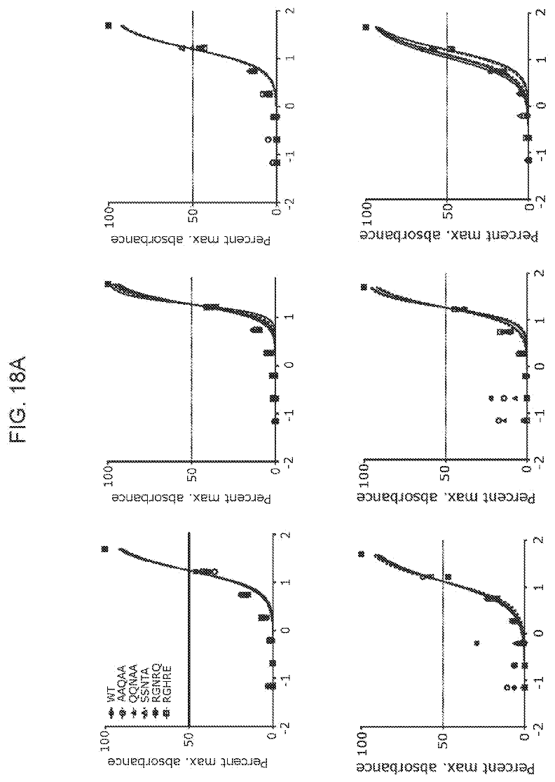

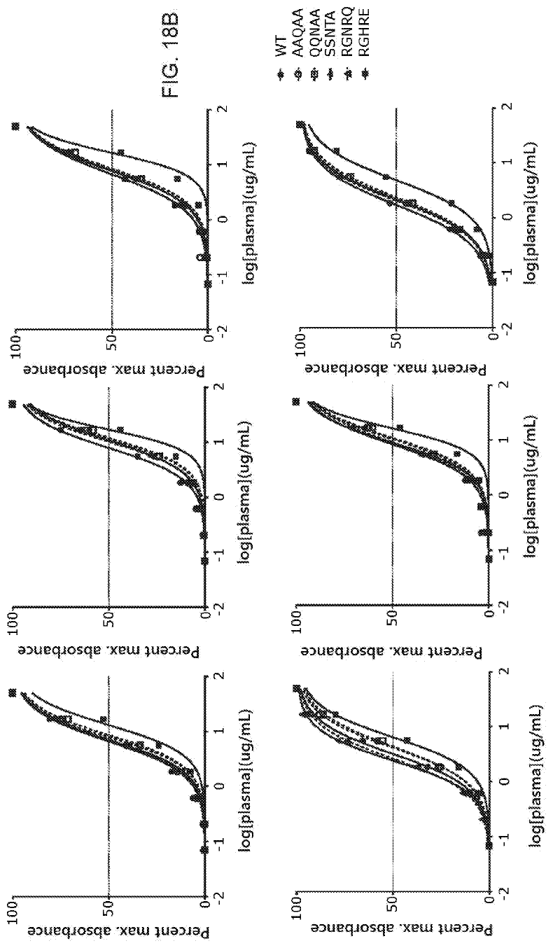

To evaluate the viability of these mutants as AAV9-like gene therapy vectors, we injected C57BL/6 mice intravenously with 1e11 genome copies (GC)/mouse of AAV9.WT.CMV.LacZ or the AAV9 mutant vectors that reduced PAV9.1 activity (n=3 per group). Biodistribution of day 14 tissue samples indicated a reduction in liver transduction for all mutants. AAV9.QQNAA performed most similarly to AAV9.WT with 17-fold fewer GC/μg DNA, whereas AAV9.RGHRE transduced liver the least efficiently with 1,110-fold fewer GC/μg DNA (FIG. 17A). However, in other organs such as heart and brain, the majority of mutants maintained near AAV9.WT levels of transduction, with the exception of the AAV2-like mutants, AAV9.RGNRQ and AAV9.RGHRE. While these differences in tissue GCs were not statistically significant, the observed trends suggest that these residues are important for AAV9 liver tropism but play less of a role in the transduction of other tissues, as most mutants displayed a “liver-detargeting” phenotype. These results were further reflected in the expression of beta-galactosidase (0-gal) in liver and heart; liver β-gal activity was highest in animals receiving AAV9.WT, whereas heart β-gal activity was similar between AAV9.WT and most mutants (with the exception of the AAV2-like mutants) (FIG. 17B and FIG. 17C).

We repeated these experiments at a ten-fold higher dose (1e12 GC/mouse) for a representative subset of AAV9 mutant vectors. Although transduction differences did not reach significance at this dose, the tissue tropism trends were consistent with those observed at the lower dose, particularly for heart and muscle samples (FIG. 17D). Again, these results were reflected in β-gal activity in histological sections of liver, heart, and muscle (FIG. 17E-FIG. 17G).

7. Epitope-Based Mutations in AAV9 do not Significantly Affect Binding or Neutralization by Polyclonal Plasma or Sera

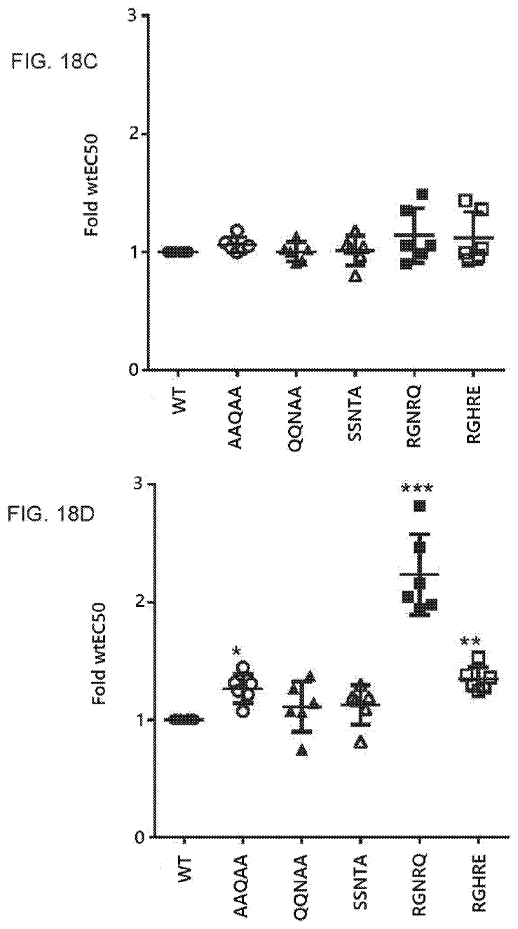

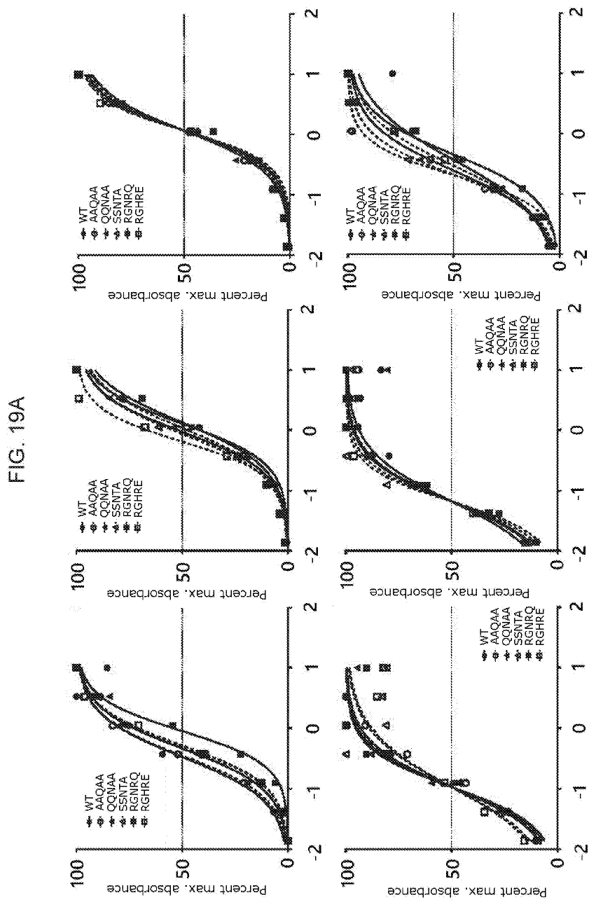

We next assessed the ability of PAV9.1 epitope-based mutant vectors to evade the binding of and neutralization by polyclonal plasma or sera. We first utilized plasma from C57BL/6 mice previously injected intravenously with AAV9.WT (7.5e8 or 7.5e9 GC/mouse, n=6 per group). We determined the dilution of plasma required to reach half-maximal binding. Binding of plasma from low-dose mice to mutant vectors was almost indistinguishable from binding to AAV9.WT (FIG. 18A-FIG. 18C). In contrast, we observed significant differences in the EC50 of plasma from high-dose mice for a subset of mutants, most notably AAV9.RGNRQ, relative to the EC50 for AAV9.WT (FIG. 18B-FIG. 18D). Despite an average two-fold increase in the EC50 of high-dose mouse plasma for AAV9.RGNRQ, we did not observe a reduction of the effective NAb titer of the plasma in this mutant (data not shown).

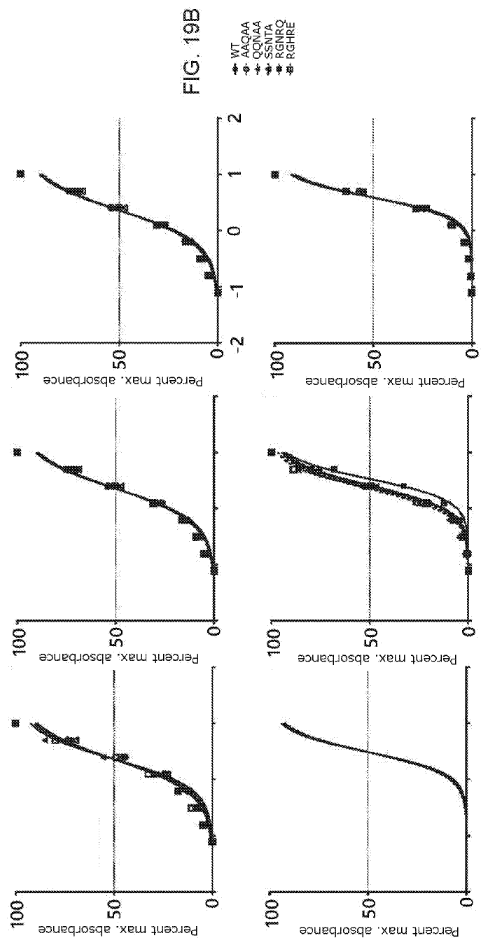

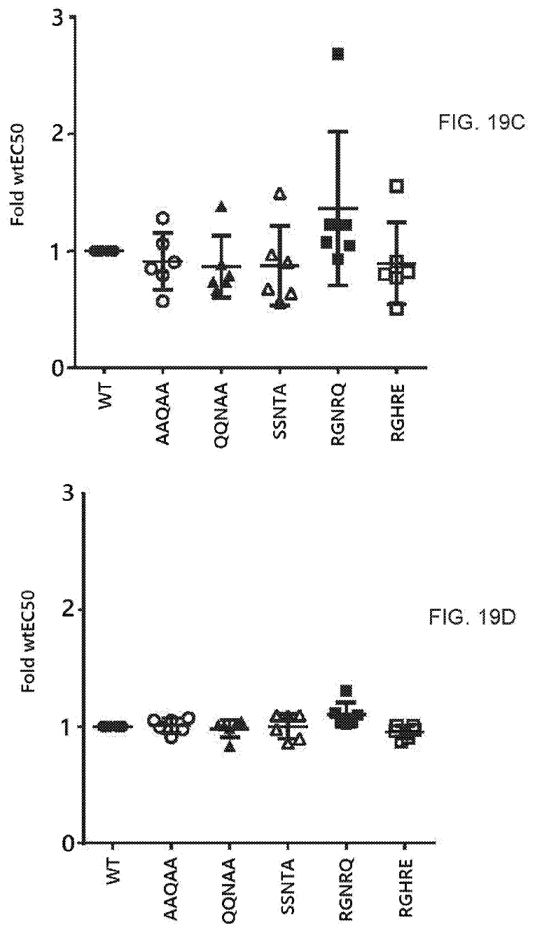

To determine if this trend in EC50 increase was true for non-human primate samples, we obtained sera from a panel of six macaques that received AAV9 vector or a novel vector closely related to AAV9 with the same VP3 sequence (2 amino acid difference in the non-structural VP1 region). We confirmed that the macaques had NAb titers against AAV9 of <1:5 (defined as NAb negative) prior to administration. Although we did observe some variation in the EC50s of each animal's serum for mutant vectors when compared to the EC50 for AAV9.WT, no clear trend of increased or decreased binding emerged based on mutant identity (FIG. 19A and FIG. 19C). When testing sera from macaques with pre-existing NAb titers against AAV9 (attributed to a prior AAV infection), we observed little to no variation in the EC50 of the sera for the panel of AAV9 mutants (FIG. 19B and FIG. 19D). This was in stark contrast to the variations seen in the EC50 of injected sera, suggesting fundamental differences between the relevant anti-AAV epitope repertoire of serum generated in response to AAV infection and AAV vector administration. Additionally, the increase in EC50 of injected non-human primate sera for AAV9.RGNRQ did not decrease the effective NAb titer of the sera for AAV9.RGNRQ (data not shown).

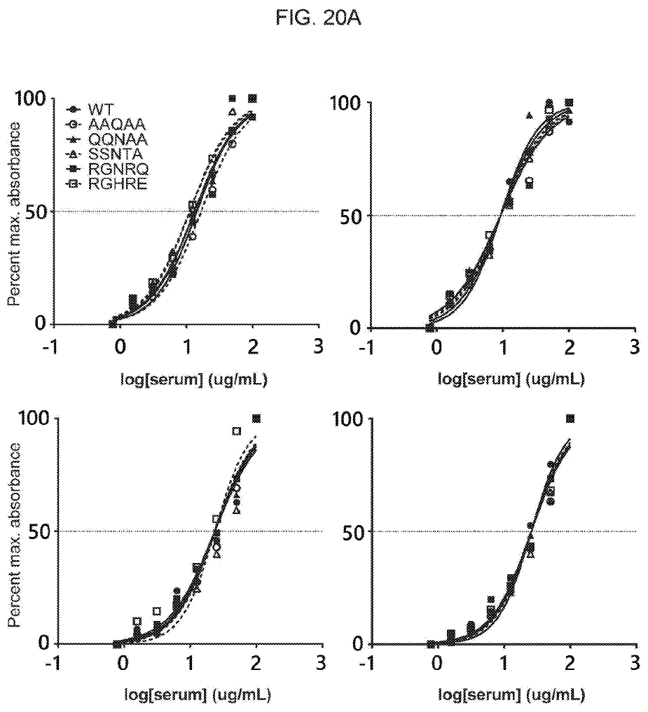

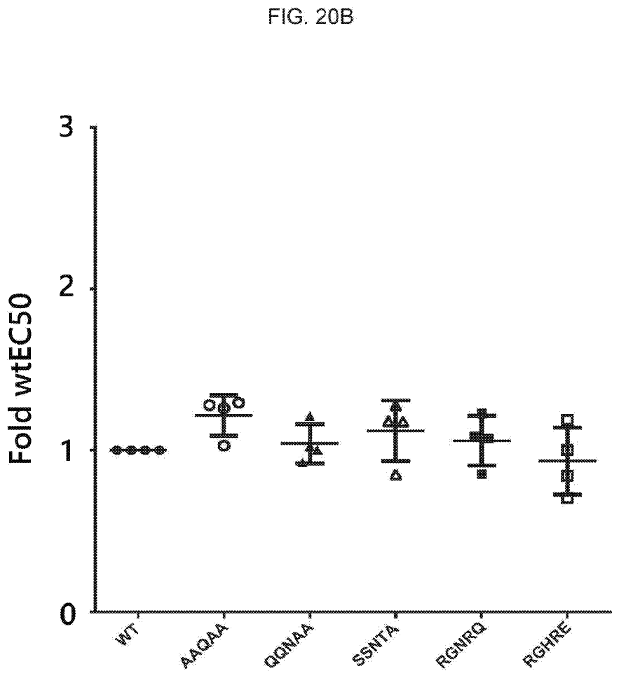

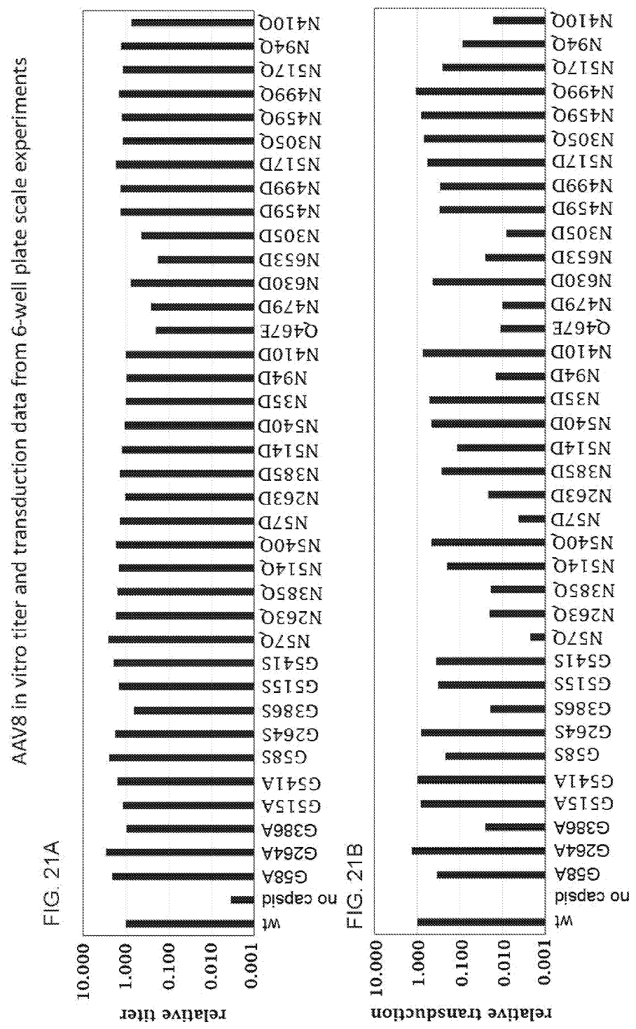

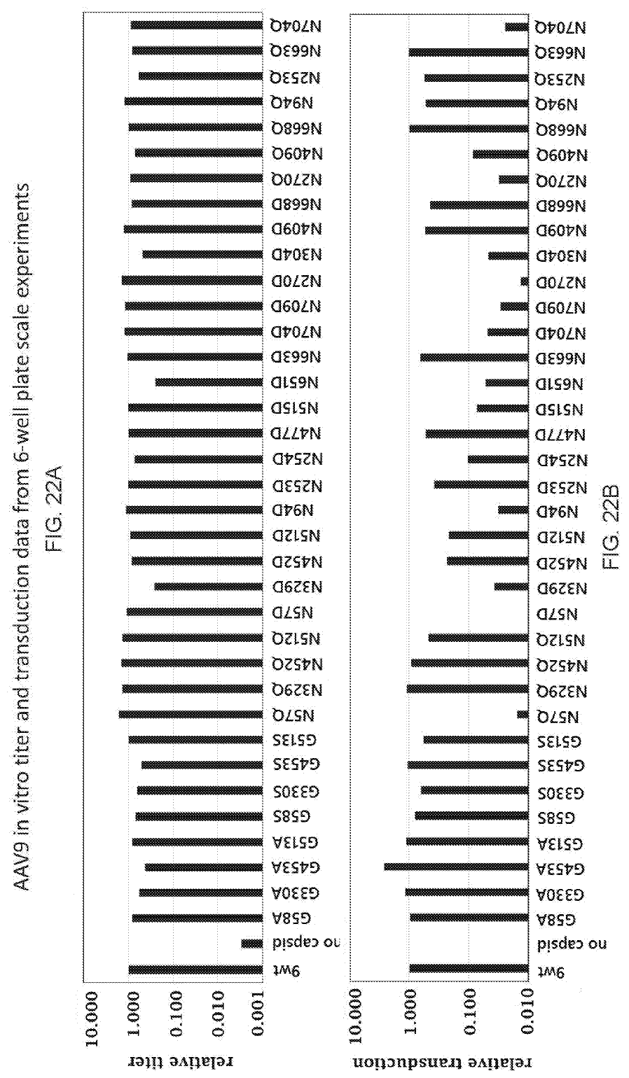

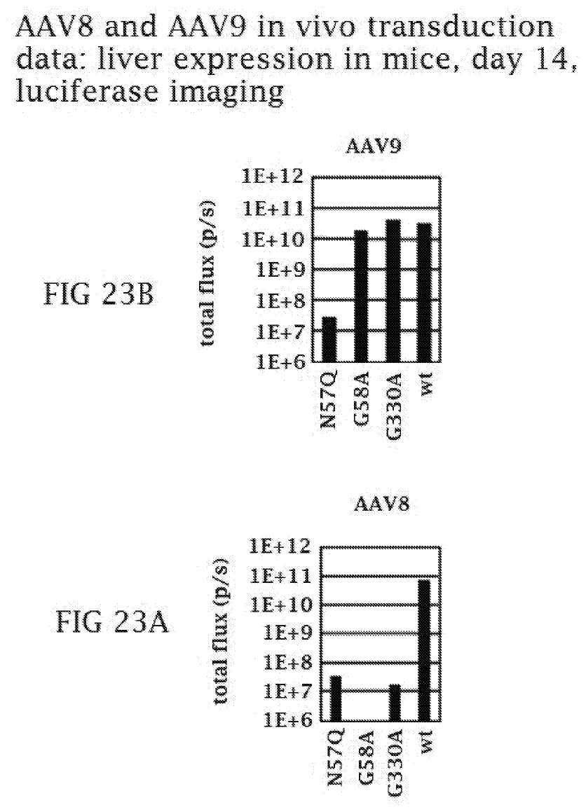

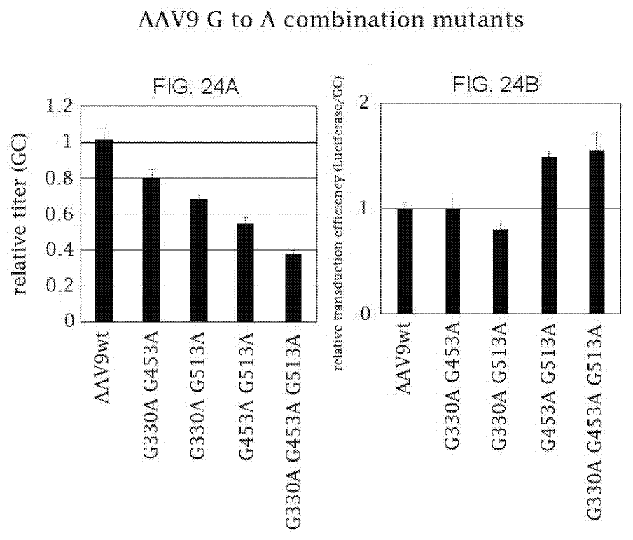

Finally, we assessed NAb-positive serum samples from four normal human donors for binding to AAV9.WT and mutant vectors. As was the case for the uninjected, NAb-positive non-human primate serum samples, all four NAb-positive normal human donor samples demonstrated minimal variation in EC50 for AAV9 mutant versus WT vectors (FIG. 20A-FIG. 20B). As expected, the lack of changes in EC50 for the mutant vectors translated to a lack of reduction in NAb titer of sera toward AAV9 mutant vectors (data not shown).

C. Discussion

Here, we report the cryo-reconstruction of AAV9 in complex with the highly potent and specific mAb PAV9.1. The epitope determined for PAV9.1 largely overlaps with the epitopic regions of other AAV NAbs isolated from mouse hybridomas, namely ADK8 (AAV8; 586-LQQQNT-591), E4E (AAV1; 492-TKTDNNN-498), 5H7 (AAV1; 496-NNNS-499, 588-STDPATGD-595), and C37 (AAV2; 492-SADNNNS-498, 585-RGNRQ-589) (Gurda B L, et al. J Virol. 2012; 86(15):7739-51; Gurda B L, et al. J Virol. 2013; 87(16):9111-24; Tseng Y S, et al. J Virol. 2015; 89(3):1794-1808). Thus, despite the large degree of sequence and structural variations among the serotypes in HVR V and VIII, this finding suggests that the three-fold protrusions may be a significant site of AAV9 neutralization as it is for other serotypes. Previous findings regarding the repertoire of NAbs directed against other AAV capsids may therefore be applicable to AAV9. Although the various mapped neutralizing epitopes show overlap, the binding angles and orientations of the NAbs vary significantly. When bound to AAV9, PAV9.1 extends into the center of the three-fold axis of symmetry, sterically limiting the occupancy to 20 Fab particles; in contrast, mAbs raised against other serotypes bind on the top or face outward from the three-fold axis, allowing higher occupancy. Studies have identified both HVR V and VIII as shared antigenic regions across serotypes, including AAV2 (in complex with C37B, 11 Å), AAV8 (in complex with ADK8, 18.7 Å), and AAV1 (in complex with 5H7, 23 Å), which bears the most similarity to the binding footprint of PAV9.1 for AAV9 (Gurda B L, et al. J Virol. 2012; 86(15):7739-51; Gurda B L, et al. J Virol. 2013; 87(16):9111-24; Tseng Y S, et al. J Virol. 2015; 89(3):1794-1808). Therefore, the structure reported here is similar to lower-resolution structures previously reported for other AAV serotypes.

HVR VIII serotype swaps conferred varying degrees of binding and neutralization evasion to their corresponding mutant vectors. Swapping this region with the AAV2-based RGHRE motif, the most divergent mutant from the WT.AAV9 sequence, ablated PAV9.1 neutralization at all dilutions tested. Thus, engineering only five amino acids in the capsid can evade a monoclonal Nab. In fact, the minimal change required to significantly reduce PAV9.1 activity was a single amino acid substitution, with even a conserved amino acid leading to ablation of both binding and neutralization. Mutations in the NNN motif in HVR V reduced PAV9.1's ability to bind and neutralize AAV9 despite having high conservation among serotypes, indicating that it is also an integral part of the PAV9.1 epitope.

We observed a strong correlation between a reduction in PAV9.1's binding to a given AAV9 mutant and its ability to block transduction of that mutant in vitro, suggesting that the relative strength of an NAb to AAV is correlated with the NAb's neutralizing ability. However, data from our lab and others suggest that the binding antibody titer against AAV is not always a good predictor of an individual's NAb titer, as some individuals have moderate binding titers against AAV but are NAb negative (Falese L, et al. Gene Ther. 2017; 24(12):768-78; Huttner N A, et al. Gene Ther. 2003; 10(26):2139-47) (unpublished data). Despite these findings, the exclusion criteria of some clinical trials include not only NAb titer but also binding titer (George L A, et al. Blood. 2017; 130(Suppl 1):604; Mendell J R, et al. N Engl J Med. 2017; 377(18):1713-22). Therefore, epitope mapping studies are critical for identifying the features of binding epitopes and determining if they share any commonalities with neutralizing epitopes. Shared motifs would suggest that strength of binding, rather than interactions with specific residues, plays a large role in AAV neutralization, thus allowing researchers to focus simply on reducing the binding of NAbs. Disparate motifs, however, would suggest that neutralization is more a function of binding location rather than strength of binding and indicate that researchers should focus on ablating NAb binding to these unique regions.

Although the mutations in the AAV9 vectors dramatically reduced binding and neutralization by a purified monoclonal PAV9.1 antibody, these mutations did not significantly evade binding or neutralization by polyclonal antibodies from serum or plasma of mice, macaques, or human donors that were previously exposed to AAV. Most notably, plasma from mice that received the higher intravenous dose of AAV9 vector bound the RGNRQ mutant about two-fold less efficiently than WT.AAV9 vector; this change was much more modest than the 50-fold reduction observed with PAV9.1 mAb. Even though the QQNAA, SSNTA, and RGHRE mutations had a greater impact on PAV9.1 binding and neutralization than the RGNRQ mutation, the polyclonal plasma bound these mutants in the same manner as WT.AAV9. This result suggests that while the 586-SAQAQ-590 motif is a potent neutralizing epitope and mutations in this region can block PAV9.1 activity, in vitro activity against a mAb does not predict activity against polyclonal antibodies. Perhaps surprisingly, the RGNRQ mutant efficiently blocked binding of AAV9 antibodies by using the three-fold protrusions. This result clearly shows that not all mutations behave the same against polyclonal responses and that a larger repertoire of antibodies utilize this region for binding.

Despite the reduction in polyclonal binding, the RGNRQ mutant vector did not evade the polyclonal NAb response generated by these mice in response to vector administration. As expected, mutants that did not reduce binding to the polyclonal plasma also did not evade neutralization. Given that the nearly 100-fold increase in the EC50 of PAV9.1 for RGNRQ relative to WT.AAV9 resulted in only an eight-fold decrease in PAV9.1 neutralizing titer, it was not surprising that a two-fold increase in the EC50 of polyclonal plasma for RGNRQ did not reduce the neutralizing titer. Although studies show that most the majority of mapped AAV epitopes lie on the three-fold axis and that HVR VIII is implicated in the mapped epitopes for most serotype-specific NAbs, we were surprised to find that none of the mutations tested in this region dramatically affected polyclonal activity (it should be noted that the mapped epitopes may not be representative of the complete repertoire, as the total number of mapped epitopes is small and the exact screening and selection methods for some studies are unknown).

Tse and colleagues recently used a library approach to combine the epitopes of three different NAbs identified against AAV1 and generate a novel AAV1-based capsid, with over 20 amino acid changes from the parental AAV1. This capsid could evade not only anti-AAV1 monoclonal NAbs but also polyclonal samples from AAV vector-injected mice and non-human primates in addition to polyclonal samples from normal human donors exposed to AAV (Tse L V, et al. Proc Natl Acad Sci USA. 2017; 114(24), E4812-21). This suggests that neutralizing epitopes may overlap following vector exposure and viral infection, but this repertoire is subtly diverse. In other words, the total number of residues that require modification to confer binding and neutralizing evasion to AAV is more extensive than previously thought. Engineering novel capsids that can address both scenarios may require combinatorial and high-throughput approaches.

This study explored whether vectors engineered to evade a pre-existing NAb response from a prior AAV infection would also function in a re-administration setting. The polyclonal samples for which the PAV9.1-based AAV9 mutant vectors demonstrated even minimal evasion were acquired from sources that had received AAV vector and not from sources that were previously infected with AAV. Whereas injected samples demonstrated modestly variable binding curves for the panel of AAV9 mutants, the binding curves generated by vector-naïve but virally exposed sources were similar to the curves of WT.AAV9. These discrepancies highlight the fundamental differences among the AAV antibody repertoire generated in response to vector administration or infection.

Historically, naïve subjects injected with AAV vector generate an NAb response that is specific to the vector administered or limited to closely related serotypes (Flotte T R, et al. Hum Gene Ther. 2011; 22(10):1239-47) (unpublished data). Most macaque studies and gene therapy clinical trials have shown a similar result (Greig J A, et al. Vaccine. 2016; 34(50):6323-29; Greig J A, et al. Hum Gene Ther Clin Dev. 2017; 28(1):39-50) (unpublished data). In stark contrast, subjects with pre-existing antibodies for one AAV serotype are almost always seropositive for and have NAbs against the majority of other serotypes, even those that are distantly related (Calcedo R and Wilson J M. Hum Gene Ther Clin Dev. 2016; 27(2):79-82; Flotte T R, et al. Hum Gene Ther. 2011; 22(10):1239-47; Harrington E A, et al. Hum Gene Ther. 2016; 27(5):345-53) (unpublished data). To date, all novel mapped AAV mAbs are specific for an individual serotype and cross-react only with closely related serotypes (for example, 5H7 binding to both AAV1 and AAV6); no previously isolated neutralizing AAV mAbs recapitulate the broader responses commonly seen following AAV infection (Gurda B L, et al. J Virol. 2013; 87(16):9111-24). Therefore, further studies are necessary to identify motifs that comprise broadly neutralizing epitopes relevant to pre-existing immunity, determine if the epitopes overlap with serotype-specific epitopes, and evaluate how the overlapping motifs confer a broadly neutralizing phenotype to the NAbs.

The magnitude of an NAb response varies widely between methods of exposure; rarely does an individual with natural immunity have an NAb titer exceeding 1:80 (humans) or 1:320 (macaques); in contrast, NAb titers >1:1,000 can easily be achieved in response to the delivery of a modest dose of vector (Greig J A, et al. Vaccine. 2016; 34(50):6323-29; Greig J A, et al. Hum Gene Ther Clin Dev. 2017; 28(1):39-50; Greig J A, et al. PLoS One. 2014; 9(11):e112268). In this study, mice receiving the highest vector dose resulting in the highest NAb titers had measurable variations in mutant vector binding; this suggests that the strength of an NAb response impacts mutant efficiency. Often, studies aim to reduce an individual's NAb titer to below the threshold that interferes with gene transfer (1:10 for intravenous administration) (Chicoine L G, et al. Mol Ther. 2014; 22(2):338-47; Wang L, et al. Hum Gene Ther. 2011; 22(11):1389-1401). Mutant capsids engineered based on a single neutralizing epitope that only confer evasion to high titer sera would not significantly increase the number of individuals eligible to receive AAV gene therapy, as the lower titers are still above the threshold at which transduction is appreciably inhibited.

The minimal mutation required to reduce PAV9.1 binding at Q590 in HVR VIII conferred a liver-detargeting phenotype to the resulting mutants, even following a conservative amino acid substitution to asparagine. Mutations in the HVR V portion of the epitope also reduced liver transduction. These results are in agreement with previous observations that these residues in HVR V and VIII play integral roles in liver transduction, as well as previous reports of mapped neutralizing AAV epitopes that show overlap with regions essential for gene transfer (Adachi K, et al. Nature Communications. 2014; 5: 3075; Tseng T S, et al. J Virol. 2015; 89(3):1794-808). This suggests that it would be difficult to engineer a mutant that can evade NAbs while maintaining the parental transduction profile. For some indications in heart and muscle, where liver transduction may be less consequential, this modification in tropism may be acceptable. Notably, the majority of mutants maintained WT.AAV9 levels of transduction in peripheral organs at both doses.

Whereas the RGNRQ mutant demonstrated modest binding modifications in the presence of polyclonal antibodies, it displayed an AAV2-like transduction profile: poorly transducing not just liver but all peripheral organs. Taken together, these data indicate the importance of integrating knowledge about a mapped neutralizing epitope with available information about AAV functional domains. Generating a capsid that can evade NAbs is not sufficient, as the capsid is only useful if it can still perform its primary function of target tissue transduction. Recent studies have used this strategy to incorporate multiple epitopes of AAV1 to generate AAV1-based vectors that can evade NAbs while maintaining AAV1-like transduction profiles (Tse L V, et al. Proc Natl Acad Sci USA. 2017; 114(24), E4812-21).

In summary, this study provides critical information regarding the design of AAV9-based vectors able to evade humoral immune responses. Future studies are required to further understand the complexity of the NAb response to AAV9 vectors to inform the design of next-generation capsids.

### Sequence Listing Free Text

The following information is provided for sequences containing free text under numeric identifier <223>.

| SEQ ID NO: | |
|---|---|
| (containing | |
| free text) | Free text under <223> |
| 9 | <223> Synthetic Construct |
| 20 | <223> AAV mutant 8G264AG515A |
| 21 | <223> Synthetic Construct |
| 22 | <223> AAV mutant 8G264AG541A |
| 23 | <223> Synthetic Construct |
| 24 | <223> AAV mutant 8G515AG541A |
| 25 | <223> Synthetic Construct |
| 26 | <223> AAV mutant 8G264AG515AG541A |
| 27 | <223> Synthetic Construct |
| 28 | <223> AAV mutant 9G330AG453A |
| 29 | <223> Synthetic Construct |
| 30 | <223> AAV mutant 9G330AG513A |
| 31 | <223> Synthetic Construct |
| 32 | <223> AAV mutant 9G453AG513A |
| 33 | <223> Synthetic Construct |
| 34 | <223> AAV mutant 9G330AG453AG513A |
| 35 | <223> Synthetic Construct |
| 38-110 | <223> primer sequence |
| 113 | <223> AAVhu68 vp1 capsid of Homo Sapiens origin |
| 114 | <223> Synthetic Construct |
| 115 | <223> AAV8 G264A/G541A/N499Q |
| 116 | <223> AAV8 G264A/G541A/N459Q |
| 117 | <223> AAV8 G264A/G541A/N305Q/N459Q |
| 118 | <223> AAV8 G264A/G541A/N305Q/N499Q |
| 119 | <223> AAV8 G264A/G541A/N459Q/N499Q |
| 120 | <223> AAV8 G264A/G541A/N305Q/N459Q/N499Q |

All documents cited in this specification are incorporated herein by reference. U.S. Provisional Patent Application Nos. 62/722,388 and 62/722,382, both filed Aug. 24, 2018, U.S. Provisional Patent Application Nos. 62/703,670 and 62/703,673, both filed Jul. 26, 2018, U.S. Provisional Patent Application Nos. 62/677,471 and 62/677,474, both filed May 29, 2018, U.S. Provisional Patent Application No. 62/667,585, filed May 29, 2018, and U.S. Provisional Patent Application No. 62/635,964, filed Feb. 27, 2018 are incorporated herein by reference. U.S. Provisional Patent Application No. 63/667,881, filed May 7, 2018, U.S. Provisional Patent Application No. 62/667,888, filed May 7, 2018, U.S. Provisional Patent Application No. 62/667,587, filed May 6, 2018, U.S. Provisional Patent Application No. 62/663,797, filed Apr. 27, 2018, U.S. Provisional Patent Application No. 62/663,788, filed Apr. 27, 2018, U.S. Provisional Patent application No. 62/635,968, filed Feb. 27, 2018 are incorporated by reference. The SEQ ID NO which are referenced herein and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

1. A composition comprising a mixed population of recombinant adeno-associated virus (rAAV), each of said rAAV comprising:
(a) an AAV capsid comprising a combined total of about 60 capsid vp1 proteins, vp2 proteins and vp3 proteins, wherein the vp1, vp2 and vp3 proteins are:
a heterogeneous population of vp1 proteins which are produced from a nucleic acid sequence encoding a selected AAV vp1 amino acid sequence,
a heterogeneous population of vp2 proteins which are produced from a nucleic acid sequence encoding a selected AAV vp2 amino acid sequence, and
a heterogeneous population of vp3 proteins which are produced from a nucleic acid sequence encoding a selected AAV vp3 amino acid sequence,
wherein: the heterogenous population of vp1, vp2 and vp3 proteins contain subpopulations with amino acid modifications comprising at least two highly deamidated asparagines in asparagine-glycine pairs in the AAV capsid and optionally further comprising subpopulations comprising other deamidated amino acids, wherein the deamidation results in an amino acid change, provided that the rAAV is not AAVhu68; and
(b) a vector genome in the AAV capsid, wherein the vector genome comprises a nucleic acid molecule comprising AAV inverted terminal repeat sequences and a non-AAV nucleic acid sequence encoding a product operably linked to sequences which direct expression of the product in a host cell.

2. The composition according to claim 1, wherein the deamidated asparagines are deamidated to an aspartic acid, an isoaspartic acid, an interconverting aspartic acid/isoaspartic acid pair, or combinations thereof.

3. The composition according to claim 1, wherein the capsid further comprises deamidated glutamine(s) which are deamidated to (α)-glutamic acid, γ-glutamic acid, an interconverting (α)-glutamic acid/γ-glutamic acid pair, or combinations thereof.

4. The composition according to claim 1, wherein the capsid comprises four to five highly deamidated asparagines which are in asparagine-glycine pairs.

5. The composition according to claim 1, wherein the capsid comprises 65% to 100% deamidated asparagine at position 57, relative to the numbering of AAV8 or AAV9, as determined using mass spectrometry.

6. The composition according to claim 1, comprising:
(a) rAAV with an AAV8 capsid, said composition further comprising a subpopulation in which at least 70% to 100% of the N in the capsid are deamidated at positions: N57, N263, N385, N514, and/or N540 of SEQ ID NO: 6 (encoded AAV8 vp1], based on the numbering of the AAV8 vp1, with the initial methionine (M);
(b) rAAV with an AAV9 capsid, further comprising a subpopulation in which at least 65% to 100% of the N in the capsid are deamidated at positions: N57, N329, N452, and/or N512, based on the numbering of the SEQ ID NO: 7 (encoded AAV9 vp1), with the initial M;
(c) rAAV with an AAVrh10 capsid (AAVrh10), further comprising a subpopulation of vp1, vp2 and/or vp3 which are at least 70% to 100% N deamidated at the N-G pair at one or more of positions N263, N385, and/or N514, based on the numbering of SEQ ID NO: 112 (encoded AAVrh10 vp1), with the initial M, or
(d) rAAV with an AAVhu37 capsid (AAVhu37), further comprising a subpopulation of vp1, vp2 and/or vp3 which are at least 70% to 100% N deamidated at the N-G pair at one or more of positions N263, N385, and/or N514, based on the numbering of SEQ ID NO: 36 (encoded AAVhu37 vp1), with the initial M.

7. The composition according to claim 1, wherein the composition comprises:
(a) rAAV with an AAV1 capsid comprising a subpopulation of vp1, vp2 and/or vp3 which are at least 70% to 100% N deamidated at the N-G pair at one or more of positions: N57, N383, N512, N718, based on the numbering of SEQ ID NO: 1, based on the numbering of the predicted vp1 amino acid sequence with the initial M;
(b) rAAV with an AAV3B capsid comprising a subpopulation of vp1, vp2 and/or vp3 which are at least 70% to 100% N deamidated at the N-G pair at one or more of positions: N57, N382, N512, N718, with reference to the numbering of SEQ ID NO: 2, based on the numbering of the predicted vp1 amino acid sequence with the initial M;
(c) rAAV with an AAV5 capsid comprising a subpopulation of vp1, vp2 and/or vp3 which are at least 70% to 100% N deamidated at the N-G pair at one or more of positions: N56, N347, N347, N509, with reference to the numbering of SEQ ID NO: 3, based on the numbering of the predicted vp1 amino acid sequence with the initial M;
(d) rAAV with an AAV7 capsid comprising a subpopulation of vp1, vp2 and/or vp3 which are at least 70% to 100% N deamidated at the N-G pair at one or more of positions: N41, N57, N384, N514, with reference to the numbering of SEQ ID NO: 4, based on the numbering of the predicted vp1 amino acid sequence with the initial M;
(e) rAAV with an AAVrh32.33 capsid comprising a subpopulation of vp1, vp2 and/or vp3 which are at least 70% to 100% N deamidated at the N-G pair at one or more of positions: N57, N264, N292, N318, with reference to the numbering of SEQ ID NO: 5, based on the numbering of the predicted vp1 amino acid sequence with the initial M; or
(f) rAAV4 vectors comprising a subpopulation of vp1, vp2 and/or vp3 which are at least 70% to 100% N deamidated at the N-G pair at one or more of positions: N56, N264, N318, N546, with reference to the numbering of SEQ ID NO: 111, based on the numbering of the predicted vp1 amino acid sequence with the initial M.

8. The composition according to claim 1, wherein the capsid comprises 80% to 100% deamidated asparagine at position 57, relative to the numbering of AAV8 or AAV9.

9. The composition according to claim 1, wherein all or a subpopulation of the AAV vp1 proteins and/or vp3 proteins have a truncation of about 1 to about 5 amino acids at its N-terminus.

10. The composition according to claim 1, wherein all or a subpopulation of the AAV vp1 proteins and/or vp3 proteins have a truncation of about 1 to about 5 amino acids at its C-terminus.

11. A method for reducing deamidation of an AAV capsid, said method comprising producing an AAV capsid from a nucleic acid sequence containing modified AAV vp codons, the nucleic acid sequence comprising independently modified glycine codons at one to three of the asparagine-glycine pairs relative to a reference AAV vp1 sequence, such that the modified codon encodes an amino acid other than glycine.

12. A method for reducing deamidation of an AAV capsid, said method comprising producing an AAV capsid from a nucleic acid sequence containing modified AAV vp codons, the nucleic acid sequence comprising independently modified asparagine codons of at least one asparagine-glycine pair relative to a reference AAV vp1 sequence, such that the modified codon encodes an amino acid other than asparagine.

13. A method for increasing the titer, potency, or transduction of a recombinant AAV, said method comprising producing an AAV capsid from a nucleic acid sequence containing at least one AAV vp codon modified to change the asparagine or glycine of at least one asparagine-glycine pairs in the capsid to a different amino acid.

14. The method according to claim 11, wherein said modified codon is in a vg2 and/or vp3 region.

15. The method according to claim 11, wherein the asparagine-glycine pair in the vp1-unique region is retained in the modified rAAV.

16. The method according to claim 11, wherein a deamidation site is modified at a location other than:
(a) N57, N263, N385, N514, and/or N540 of SEQ ID NO: 6 (encoded AAV8 vp1), based on the numbering of the AAV8 vp1, with the initial M, for an AAV8 capsid;
(b) N57, N329, N452, and/or N512, based on the numbering of the SEQ ID NO: 7 (encoded AAV9 vp1), with the initial M, for an AAV9 capsid;
(c) N57, N263, N385, and/or N514, based on the numbering of SEQ ID NO: 112 (encoded AAVrh10 vp1), with the initial M, for an AAVrh10 capsid, or
(d) N57, N263, N385, and/or N514, based on the numbering of SEQ ID NO: 36 (encoded AAVhu37 vp1), with the initial M, for an AAVhu37 capsid.

17. The method according to claim 16, wherein the modified deamidation site is selected from a site on Table F, Table G, or Table H.

18. The method according to claim 11, wherein a deamidation site is modified at a location other than:
(a) N57, N383, N512, and/or N718, based on the numbering of SEQ ID NO: 1, based on the numbering of the predicted vp1 amino acid sequence with the initial M, for an AAV1 capsid;
(b) N57, N382, N512, and/or N718, with reference to the numbering of SEQ ID NO: 2, based on the numbering of the predicted vp1 amino acid sequence with the initial M, for an AAV3B capsid;
(c) N56, N347, N347, and/or N509, with reference to the numbering of SEQ ID NO: 3, based on the numbering of the predicted vp1 amino acid sequence with the initial M, for an AAV5 capsid;
(d) N41, N57, N384, and/or N514, with reference to the numbering of SEQ ID NO: 4, based on the numbering of the predicted vp1 amino acid sequence with the initial M, for an AAV7 capsid;
(e) N57, N264, N292, and/or N318, with reference to the numbering of SEQ ID NO: 5, based on the numbering of the predicted vp1 amino acid sequence with the initial M, for an AAVrh32.33 capsid; or
(f) N56, N264, N318, and/or N546, with reference to the numbering of SEQ ID NO: 111, based on the numbering of the predicted vp1 amino acid sequence with the initial M, for an AAV4 capsid.

19. The method according to claim 18, wherein the modified deamidation site is selected from a site on Table A, Table B, Table C, Table D, Table E, Table F, Table G, or Table H.

20. The method according to claim 11, wherein each modified codon encodes a different amino acid.

21. The method according to claim 11, wherein two or more modified codons encode the same amino acid.

22. A mutant rAAV comprising an AAV capsid with reduced deamidation as compared to an unmodified AAV capsid, which is produced using the method according to claim 11.

23. The mutant rAAV according to claim 22, having a mutant AAV capsid having capsid proteins with one or more of the following substitutions, based on the numbering of VP1:
(a) AAV8 G264A/G541A (SEQ ID NO: 23);
(b) AAV8 G264A/G541A/N499Q (SEQ ID NO: 115);
(c) AAV8 G264A/G541A/N459Q (SEQ ID NO: 116);
(d) AAV8 G264A/G541A/N305Q/N459Q (SEQ ID NO: 117);
(e) AAV8 G264A/G541A/N305Q/N499Q (SEQ ID NO: 118);
(f) AAV8 G264A/G541A/N459Q/N499Q (SEQ ID NO: 119);
(g) AAV8 G264A/G541A/N305Q/N459Q/N499Q (SEQ ID NO: 120);
(h) AAV8 G264A/G515A (SEQ ID NO: 21);
(i) AAV8G515A/G541A (SEQ ID NO: 25);
(j) AAV8 G264A/G515A/G541A (SEQ ID NO: 27);
(k) AAV9 G330/G453A (SEQ ID NO: 29);
(l) AAV9G330A/G513A (SEQ ID NO: 31);
(m) AAV9G453A/G513A (SEQ ID NO 33), and/or
(n) G330/G453A/G513A (SEQ ID NO: 35).

24. The mutant rAAV according to claim 22, having a mutant AAV capsid having capsid proteins with one or more of the following substitutions, based on the numbering of the AAV8 VP1: N263A, N514A, or AAV N540A.

25. The mutant rAAV according to claim 22, having a mutant AAV capsid having capsid proteins, wherein the wild-type NG pairs at the following positions are retained: N57, N94, N263, N305, G386, Q467, N479, and/or N653.

26. A composition comprising a population of rAAV having increased titer, potency, or transduction, said composition comprising rAAV having capsids which are modified to have decreased total deamidation as compared to an rAAV with a deamidation pattern with a capsid deamidation pattern according to any one of Table A (AAV1), Table B (AAV3B), Table C (AAV5), Table D (AAV7), Table E (AAVrh32.33), Table F (AAV8), Table G (AAV9), or Table H (AAVhu37), provided that the rAAV is not AAVhu68.

27. The composition according to claim 26, wherein the rAAV has a deamidation site modified at a location other than:
(a) N57, N263, N385, N514, and/or N540 of SEQ ID NO: 6 (encoded AAV8 vp1), based on the numbering of the AAV8 vp1, with the initial M, for an AAV8 capsid;
(b) N57, N329, N452, and/or N512, based on the numbering of the SEQ ID NO: 7 (encoded AAV9 vp1), with the initial M, for an AAV9 capsid;
(c) N57, N263, N385, and/or N514, based on the numbering of SEQ ID NO: 112 (encoded AAVrh10 vp1), with the initial M, for an AAVrh10 capsid, or
(d) N57, N263, N385, and/or N514, based on the numbering of SEQ ID NO: 36 (encoded AAVhu37 vp1), with the initial M, for an AAVhu37 capsid.

28. The composition according to claim 26, wherein the rAAV has a modified amino acid sequence deamidation site is modified at a location other than:
(a) N57, N383, N512, and/or N718, based on the numbering of SEQ ID NO: 1, based on the numbering of the predicted vp1 amino acid sequence with the initial M, for an AAV1 capsid;
(b) N57, N382, N512, and/or N718, with reference to the numbering of SEQ ID NO: 2, based on the numbering of the predicted vp1 amino acid sequence with the initial M, for an AAV3B capsid;
(c) N56, N347, N347, and/or N509, with reference to the numbering of SEQ ID NO: 3, based on the numbering of the predicted vp1 amino acid sequence with the initial M, for an AAV5 capsid;
(d) N41, N57, N384, and/or N514, with reference to the numbering of SEQ ID NO: 4, based on the numbering of the predicted vp1 amino acid sequence with the initial M, for an AAV7 capsid;
(e) N57, N264, N292, and/or N318, with reference to the numbering of SEQ ID NO: 5, based on the numbering of the predicted vp1 amino acid sequence with the initial M, for an AAVrh32.33 capsid; or
(f) N56, N264, N318, and/or N546, with reference to the numbering of SEQ ID NO: 111, based on the numbering of the predicted vp1 amino acid sequence with the initial M for an AAV4 capsid.


SEQUENCE LISTING (120 sequences)

SEQ ID NO: 1 - protein, 736 aa, adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 736 aa, adeno-associated virus 3B
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 3 - protein, 724 aa, adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 4 - protein, 737 aa, adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 5 - protein, 733 aa, adeno-associated virus rh32.33
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 6 - protein, 738 aa, adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 7 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - DNA, 2211 bp, Artificial Sequence
  adeno-associated virus 8 vp1 mutant
  CDS (1)..(2211)
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctcctctggtactcatggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggtgggagtaggcctacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtgggtgataacttgcagttgtataacacggctcctggttcggtgtttgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 9 - protein, 737 aa, Artificial Sequence
  Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISSGTHGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGSRPTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVGDNLQLYNTAPGSVFVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 10 - protein, 33 aa, adeno-associated virus 9
QRVSTTVTQNNNSEFAWPGASSWALNGRNSLMN

SEQ ID NO: 11 - protein, 33 aa, adeno-associated virus 8
QRVSTTTGQNNNSNFAWTAGTKYHLNGRNSLAN

SEQ ID NO: 12 - protein, 33 aa, adeno-associated virus rh10
QRVSTTLSQNNNSNFAWTGATKYHLNGRDSLVN

SEQ ID NO: 13 - protein, 33 aa, adeno-associated virus 3B
QRLSKTANDNNNSNFPWTAASKYHLNGRDSLVN

SEQ ID NO: 14 - protein, 33 aa, adeno-associated virus 2
QRVSKTSADNNNSEYSWTGATKYHLNGRDSLVN

SEQ ID NO: 15 - protein, 33 aa, adeno-associated virus 9
YGQVATNHQSAQAQAQTGWVQNQGILPGMVWQD

SEQ ID NO: 16 - protein, 33 aa, adeno-associated virus 8
YGIVADNLQQQNTAPQIGTVNSQGALPGMVWQN

SEQ ID NO: 17 - protein, 33 aa, adeno-associated virus rh10
YGVVADNLQQQNAAPIVGAVNSQGALPGMVWQN

SEQ ID NO: 18 - protein, 33 aa, adeno-associated virus 3B
YGTVANNLQSSNTAPTTRTVNDQGALPGMVWQD

SEQ ID NO: 19 - protein, 33 aa, adeno-associated virus 2
YGSVSTNLQRGNRQAATADVNTQGVLPGMVWQD

SEQ ID NO: 20 - DNA, 2214 bp, Artificial Sequence
  AAV mutant 8G264AG515A
  CDS (1)..(2214)
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgcgacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatgcaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 21 - protein, 738 aa, Artificial Sequence
  Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNATSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNARNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 22 - DNA, 2214 bp, Artificial Sequence
  AAV mutant 8G264AG541A
  CDS (1)..(2214)
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgcgacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgcgatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 23 - protein, 738 aa, Artificial Sequence
  Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNATSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNAILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 24 - DNA, 2214 bp, Artificial Sequence
  AAV mutant 8G515AG541A
  CDS (1)..(2214)
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatgcaagaaattcattggctaatcctggcatcgctatggcaacacacaaagacgacgaggagcgtttttttcccagtaacgcgatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 25 - protein, 738 aa, Artificial Sequence
  Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNARNSLANPGIAMATHKDDEERFFPSNAILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 26 - DNA, 2214 bp, Artificial Sequence
  AAV mutant 8G264AG515AG541A
  CDS (1)..(2214)
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgcgacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatgcaagaaattcattggctaatcctggcatcgctatggcaacacacaaagacgacgaggagcgtttttttcccagtaacgcgatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 27 - protein, 738 aa, Artificial Sequence
  Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNATSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNARNSLANPGIAMATHKDDEERFFPSNAILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 28 - DNA, 2211 bp, Artificial Sequence
  AAV mutant 9G330AG453A
  CDS (1)..(2211)
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatgcagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacgcttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 29 - protein, 736 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNAVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINASGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - DNA, 2211 bp, Artificial Sequence
  AAV mutant 9G330AG513A
  CDS (1)..(2211)
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatgcagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatgcacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 31 - protein, 736 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNAVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNARNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - DNA, 2211 bp, Artificial Sequence
  AAV mutant 9G453AG513A
  CDS (1)..(2211)
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacgcttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatgcacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 33 - protein, 736 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINASGQNQQTLKFSVAGPSNMAVQGRNYIPGPSYRQQRVSTTVTQNNNSEFAWPGASSWALNARNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - DNA, 2211 bp, Artificial Sequence
  AAV mutant 9G330AG453AG513A
  CDS (1)..(2211)
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatgcagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacgcttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatgcacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 35 - protein, 736 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNAVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINASGQNQQTLKFSVAGPSNMAVQGRNYIPGPSYRQQRVSTTVTQNNNSEFAWPGASSWALNARNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 36 - protein, 738 aa, adeno-associated virus hu37
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 37 - DNA, 2217 bp, adeno-associated virus hu37
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttggtcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatatccaatgggacatcgggaggaagcaccaacgacaacacctacttcggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggacgtcttcatgattccccagtacggctaccttacactgaacaatggaagtcaagccgtaggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcatcgaccagtacctgtactacttatccagaactcagtccacaggaggaactcaaggtacccagcaattgttattttctcaagctgggcctgcaaacatgtcggctcaggctaagaactggctacctggaccttgctaccggcagcagcgagtctctacgacactgtcgcaaaacaacaacagcaactttgcttggactggtgccaccaaatatcacctgaacggaagagactctttggtaaatcccggtgtcgccatggcaacccacaaggacgacgaggaacgcttcttcccgtcgagtggagtcctgatgttcggaaaacagggtgctggaagagacaatgtggactacagcagcgttatgctaaccagcgaagaagaaattaaaaccactaaccccgtagccacagaacaatacggtgtggtggctgacaacttgcagcaaaccaatacagggcctattgtgggaaatgtcaacagccaaggagccttacctggcatggtctggcagaaccgagacgtgtacctgcagggtcccatctgggccaagattcctcacacggacggcaacttccacccttcaccgctaatgggaggatttggactgaagcacccacctcctcagatcctgatcaagaacacgccggtacctgcggatcctccaacaacgttcagccaggcgaaattggcttccttcattacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttacctcacccgtaatctgtaa

SEQ ID NO: 38 - DNA, 38 bp, Artificial Sequence
  primer sequence
cgacaaccgggcaaaaccagaatagcaactttgcctgg

SEQ ID NO: 39 - DNA, 38 bp, Artificial Sequence
  primer sequence
ccaggcaaagttgctattctggttttgcccggttgtcg

SEQ ID NO: 40 - DNA, 36 bp, Artificial Sequence
  primer sequence
gacaaccgggcaaaacgacaatagcaactttgcctg

SEQ ID NO: 41 - DNA, 36 bp, Artificial Sequence
  primer sequence
caggcaaagttgctattgtcgttttgcccggttgtc

SEQ ID NO: 42 - DNA, 29 bp, Artificial Sequence
  primer sequence
ggaggcacggcacagacgcagactctggg

SEQ ID NO: 43 - DNA, 29 bp, Artificial Sequence
  primer sequence
cccagagtctgcgtctgtgccgtgcctcc

SEQ ID NO: 44 - DNA, 30 bp, Artificial Sequence
  primer sequence
caggaggcacggcagatacgcagactctgg

SEQ ID NO: 45 - DNA, 30 bp, Artificial Sequence
  primer sequence
ccagagtctgcgtatctgccgtgcctcctg

SEQ ID NO: 46 - DNA, 29 bp, Artificial Sequence
  primer sequence
ctcctcccgatgtcgcgttggagatttgc

SEQ ID NO: 47 - DNA, 29 bp, Artificial Sequence
  primer sequence
gcaaatctccaacgcgacatcgggaggag

SEQ ID NO: 48 - DNA, 31 bp, Artificial Sequence
  primer sequence
cccacggcctgactagcgttgttgagtgtta

SEQ ID NO: 49 - DNA, 31 bp, Artificial Sequence
  primer sequence
taacactcaacaacgctagtcaggccgtggg

SEQ ID NO: 50 - DNA, 43 bp, Artificial Sequence
  primer sequence
ggattagccaatgaatttcttgcattcagatggtatttggtcc

SEQ ID NO: 51 - DNA, 43 bp, Artificial Sequence
  primer sequence
ggaccaaataccatctgaatgcaagaaattcattggctaatcc

SEQ ID NO: 52 - DNA, 39 bp, Artificial Sequence
  primer sequence
tttgccaaaaatcaggatcgcgttactgggaaaaaaacg

SEQ ID NO: 53 - DNA, 39 bp, Artificial Sequence
  primer sequence
cgtttttttcccagtaacgcgatcctgatttttggcaaa

SEQ ID NO: 54 - DNA, 27 bp, Artificial Sequence
  primer sequence
ggacccttcaacgcactcgacaagggg

SEQ ID NO: 55 - DNA, 27 bp, Artificial Sequence
  primer sequence
ccccttgtcgagtgcgttgaagggtcc

SEQ ID NO: 56 - DNA, 35 bp, Artificial Sequence
  primer sequence
tggctcctcccgatgtgctgttggagatttgcttg

SEQ ID NO: 57 - DNA, 35 bp, Artificial Sequence
  primer sequence
caagcaaatctccaacagcacatcgggaggagcca

SEQ ID NO: 58 - DNA, 33 bp, Artificial Sequence
  primer sequence
cccacggcctgactactgttgttgagtgttagg

SEQ ID NO: 59 - DNA, 33 bp, Artificial Sequence
  primer sequence
cctaacactcaacaacagtagtcaggccgtggg

SEQ ID NO: 60 - DNA, 46 bp, Artificial Sequence
  primer sequence
ttagccaatgaatttctgctattcagatggtatttggtcccagcag

SEQ ID NO: 61 - DNA, 46 bp, Artificial Sequence
  primer sequence
ctgctgggaccaaataccatctgaatagcagaaattcattggctaa

SEQ ID NO: 62 - DNA, 45 bp, Artificial Sequence
  primer sequence
ttgtttgccaaaaatcaggatgctgttactgggaaaaaaacgctc

SEQ ID NO: 63 - DNA, 45 bp, Artificial Sequence
  primer sequence
gagcgtttttttcccagtaacagcatcctgatttttggcaaacaa

SEQ ID NO: 64 - DNA, 33 bp, Artificial Sequence
  primer sequence
ctcccccttgtcgaggctgttgaagggtccgag

SEQ ID NO: 65 - DNA, 33 bp, Artificial Sequence
  primer sequence
ctcggacccttcaacagcctcgacaagggggag

SEQ ID NO: 66 - DNA, 32 bp, Artificial Sequence
  primer sequence
cagcgactcatcaacgacaactggggattccg

SEQ ID NO: 67 - DNA, 28 bp, Artificial Sequence
  primer sequence
ggaggcacggcagatacgcagactctgg

SEQ ID NO: 68 - DNA, 36 bp, Artificial Sequence; identical to SEQ ID NO: 40
  primer sequence

SEQ ID NO: 69 - DNA, 40 bp, Artificial Sequence
  primer sequence
ccatctgaatggaagagattcattggctaatcctggcatc

SEQ ID NO: 70 - DNA, 30 bp, Artificial Sequence
  primer sequence
cgaagcccaaagccgaccagcaaaagcagg

SEQ ID NO: 71 - DNA, 28 bp, Artificial Sequence
  primer sequence
gtacctgcggtatgaccacgccgacgcc

SEQ ID NO: 72 - DNA, 37 bp, Artificial Sequence
  primer sequence
gatgctgagaaccggcgacaacttccagtttacttac

SEQ ID NO: 73 - DNA, 39 bp, Artificial Sequence
  primer sequence
cagactctgggcttcagcgatggtgggcctaatacaatg

SEQ ID NO: 74 - DNA, 30 bp, Artificial Sequence
  primer sequence
ccaatcaggcaaaggactggctgccaggac

SEQ ID NO: 75 - DNA, 26 bp, Artificial Sequence
  primer sequence
cacggacggcgacttccacccgtctc

SEQ ID NO: 76 - DNA, 31 bp, Artificial Sequence
  primer sequence
gatcctgatcaaggacacgcctgtacctgcg

SEQ ID NO: 77 - DNA, 33 bp, Artificial Sequence
  primer sequence
gtacctcggacccttccagggactcgacaaggg

SEQ ID NO: 78 - DNA, 36 bp, Artificial Sequence
  primer sequence
ctacaagcaaatctcccaggggacatcgggaggagc

SEQ ID NO: 79 - DNA, 36 bp, Artificial Sequence
  primer sequence
gctacctaacactcaaccagggtagtcaggccgtgg

SEQ ID NO: 80 - DNA, 41 bp, Artificial Sequence
  primer sequence
gctgggaccaaataccatctgcagggaagaaattcattggc

SEQ ID NO: 81 - DNA, 40 bp, Artificial Sequence
  primer sequence
ggagcgtttttttcccagtcaggggatcctgatttttggc

SEQ ID NO: 82 - DNA, 32 bp, Artificial Sequence
  primer sequence
cggaatccccagttgtcgttgatgagtcgctg

SEQ ID NO: 83 - DNA, 28 bp, Artificial Sequence
  primer sequence
ccagagtctgcgtatctgccgtgcctcc

SEQ ID NO: 84 - DNA, 36 bp, Artificial Sequence; identical to SEQ ID NO: 41
  primer sequence

SEQ ID NO: 85 - DNA, 40 bp, Artificial Sequence
  primer sequence
gatgccaggattagccaatgaatctcttccattcagatgg

SEQ ID NO: 86 - DNA, 30 bp, Artificial Sequence
  primer sequence
cctgcttttgctggtcggctttgggcttcg

SEQ ID NO: 87 - DNA, 28 bp, Artificial Sequence
  primer sequence
ggcgtcggcgtggtcataccgcaggtac

SEQ ID NO: 88 - DNA, 37 bp, Artificial Sequence
  primer sequence
gtaagtaaactggaagttgtcgccggttctcagcatc

SEQ ID NO: 89 - DNA, 39 bp, Artificial Sequence
  primer sequence
cattgtattaggcccaccatcgctgaagcccagagtctg

SEQ ID NO: 90 - DNA, 30 bp, Artificial Sequence
  primer sequence
gtcctggcagccagtcctttgcctgattgg

SEQ ID NO: 91 - DNA, 26 bp, Artificial Sequence
  primer sequence
gagacgggtggaagtcgccgtccgtg

SEQ ID NO: 92 - DNA, 31 bp, Artificial Sequence
  primer sequence
cgcaggtacaggcgtgtccttgatcaggatc

SEQ ID NO: 93 - DNA, 35 bp, Artificial Sequence
  primer sequence
gcagcgactcatcaacgacaactggggattccggc

SEQ ID NO: 94 - DNA, 35 bp, Artificial Sequence
  primer sequence
gccggaatccccagttgtcgttgatgagtcgctgc

SEQ ID NO: 95 - DNA, 34 bp, Artificial Sequence
  primer sequence
cagcgactcatcaacgacaactggggattccggc

SEQ ID NO: 96 - DNA, 34 bp, Artificial Sequence
  primer sequence
gccggaatccccagttgtcgttgatgagtcgctg

SEQ ID NO: 97 - DNA, 30 bp, Artificial Sequence
  primer sequence
gcgactcatcaacgacaactggggattccg

SEQ ID NO: 98 - DNA, 30 bp, Artificial Sequence
  primer sequence
cggaatccccagttgtcgttgatgagtcgc

SEQ ID NO: 99 - DNA, 31 bp, Artificial Sequence
  primer sequence
ctctgggcttcagcgaaggtgggcctaatac

SEQ ID NO: 100 - DNA, 31 bp, Artificial Sequence
  primer sequence
gtattaggcccaccttcgctgaagcccagag

SEQ ID NO: 101 - DNA, 29 bp, Artificial Sequence
  primer sequence
cctcggacccttcgacggactcgacaagg

SEQ ID NO: 102 - DNA, 34 bp, Artificial Sequence
  primer sequence
tacaagcaaatctccgacgggacatcgggaggag

SEQ ID NO: 103 - DNA, 34 bp, Artificial Sequence
  primer sequence
ctacctaacactcaacgacggtagtcaggccgtg

SEQ ID NO: 104 - DNA, 45 bp, Artificial Sequence
  primer sequence
ctgggaccaaataccatctggatggaagaaattcattggctaatc

SEQ ID NO: 105 - DNA, 39 bp, Artificial Sequence
  primer sequence
gagcgtttttttcccagtgacgggatcctgatttttggc

SEQ ID NO: 106 - DNA, 29 bp, Artificial Sequence
  primer sequence
ccttgtcgagtccgtcgaagggtccgagg

SEQ ID NO: 107 - DNA, 34 bp, Artificial Sequence
  primer sequence
ctcctcccgatgtcccgtcggagatttgcttgta

SEQ ID NO: 108 - DNA, 34 bp, Artificial Sequence
  primer sequence
cacggcctgactaccgtcgttgagtgttaggtag

SEQ ID NO: 109 - DNA, 45 bp, Artificial Sequence
  primer sequence
gattagccaatgaatttcttccatccagatggtatttggtcccag

SEQ ID NO: 110 - DNA, 39 bp, Artificial Sequence
  primer sequence
gccaaaaatcaggatcccgtcactgggaaaaaaacgctc

SEQ ID NO: 111 - protein, 734 aa, adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 112 - protein, 738 aa, adeno-associated virus rh10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 113 - DNA, 2211 bp, adeno-associated virus hu68
  CDS (1)..(2211)
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 114 - protein, 736 aa, adeno-associated virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 115 - protein, 738 aa, Artificial sequence
  AAV8 G264A/G541A/N499Q
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNATSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNQNSNFAWTAGTKYHLNGRNSLANPGIAMATHKDDEERFFPSNAILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 116 - protein, 738 aa, Artificial sequence
  AAV8 G264A/G541A/N459Q
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNATSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTAQTQTLGFSQGGPNTMANQAKNWLPGPCYRQQRVSTTTGQNNNSNFAWTAGTKYHLNGRNSLANPGIAMATHKDDEERFFPSNAILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 117 - protein, 738 aa, Artificial sequence
  AAV8 G264A/G541A/N305Q/N459Q
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNATSGGATNDNTYFGYSTPWGYFDFNRFHCHFSPRDWQRLINQNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNQNSNFAWTAGTKYHLNGRNSLANPGIAMATHKDDEERFFPSNAILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 118 - protein, 738 aa, Artificial sequence; identical to SEQ ID NO: 117
  AAV8 G264A/G541A/N305Q/N499Q

SEQ ID NO: 119 - protein, 738 aa, Artificial sequence
  AAV8 G264A/G541A/N459Q/N499Q
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNATSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTAQTQTLGFSQGGPNTMANQAKNWLPGPCYRQQRVSTTTGQNQNSNFAWTAGTKYHLNGRNSLANPGIAMATHKDDEERFFPSNAILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 120 - protein, 738 aa, Artificial sequence
  AAV8 G264A/G541A/ N305Q/N459Q/N499Q
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNATSGGATNDNTYFGYSTPWGYFDFNRFHCHFSPRDWQRLINQNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTAQTQTLGFSQGGPNTMANQAKNWLPGPCYRQQRVSTTTGQNQNSNFAWTAGTKYHLNGRNSLANPGIAMATHKDDEERFFPSNAILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL